(19) United States
(12) Patent Application Publication
(10) Pub. No.: US 2026/0023070 A1
(43) Pub. Date: Jan. 22, 2026

(54) OPTOGENETIC ALPHA-SYNUCLEIN AGGREGATION SYSTEM-BASED COMPOUND SCREENING PLATFORM IN PD-hiPSC-mDA NEURONS

(71) Applicant: The Johns Hopkins University, Baltimore, MD (US)

(72) Inventors: Gabsang Lee, Baltimore, MD (US); Minseong Kim, Baltimore, MD (US); Eun Ra, Baltimore, MD (US); Han Seok Ko, Baltimore, MD (US); Xiaobo Mao, Baltimore, MD (US)

(21) Appl. No.: 19/106,996

(22) PCT Filed: Sep. 6, 2023
(86) PCT No.: PCT/US2023/032114

§ 371 Date: Feb. 26, 2025

Related U.S. Application Data
(63) Continuation of application No. PCT/US2022/042661, filed on Sep. 6, 2022.
(60) Provisional application No. 63/448,611, filed on Feb. 27, 2023.

(51) Int. Cl. G01N 33/50; C07K 14/435; C12N 5/0793

(52) U.S. Cl. CPC G01N 33/5058; C07K 14/435; C12N 5/0619; G01N 33/5041; C07K 2319/20; C07K 2319/60; C12N 2503/02; C12N 2510/00

(57) ABSTRACT

Provided herein are methods and compositions for identifying α-synuclein aggregation inhibitors. Also provided are methods of use of the α-synuclein aggregation inhibitors; the methods include methods of inhibition the formation of Lewi bodies and methods of treating synucleinopathies in subjects. Methods are compositions provided herein include optogenetic α-synuclein fusion proteins and an optogenetic alpha-synuclein (α-syn) aggregation system. Further, provided herein are compositions comprising α-synuclein aggregation inhibitor drug candidates identified using an optical alpha-synuclein aggregation screening system. The α-synuclein aggregation inhibitor drug candidates have neuroprotective effects in vitro and in vivo and provide proof-of principle that the optical alpha-synuclein aggregation screening system can be used to identify drug candidate for synucleopathies and tauopathies, including for example Parkinson's disease.

### CROSS-REFERENCE TO RELATED APPLICATIONS

This application claims benefit of priority under 35 U.S.C. § 119(e) of International Patent Application No. PCT/US2022/042661, filed Sep. 6, 2022, and of U.S. Provisional Application No. 63/448,611, filed Feb. 27, 2023. The disclosure of the prior applications is considered part of and are herein incorporated by reference in the disclosure of this application in its entirety.

### INCORPORATION OF SEQUENCE LISTING

The material in the accompanying sequence listing is hereby incorporated by reference into this application. The accompanying sequence listing xml file, name JHU4210_3W0, was created on Aug. 29, 2023, and is 10 kb in size.

### BACKGROUND OF THE INVENTION

### Field of the Invention

The present invention relates generally to α-synuclein protein aggregation, and more specifically to an optogenetic α-synuclein fusion protein and its use to identify α-synuclein aggregation inhibitors.

### Background Information

Parkinson's disease (PD) is a progressive, age-related neurodegenerative disease characterized by significant motor impairment. PD is mainly associated with the specific loss of midbrain dopaminergic (mDA) neurons, and it physically manifests as debilitated movement in affected individuals. The formation of unique, filamentous inclusion bodies called Lewy bodies (LBs) or Lewy neurites, comprised mostly of alpha-synuclein (α-syn) which is the product of the SNCA gene, is considered the hallmark of both PD and dementia with LBs. PD is the second most common neurodegenerative disorder, and key pathology in PD is known to be synucleinopathy; however, there is no effective cure yet. One of the major obstacles of studying PD is the inaccessibility of the brain tissue samples from PD patients. The current understanding of PD pathology has been mostly derived from postmortem brain study. Although the animal models have been very useful in exploring the pathogenesis of PD as an alternative method, they do not fully recapitulate the pathological phenotypes of human PD. Due to many possible reasons including differences of the genetic, aging, and environmental factors, the pathogenic α-syn aggregates are not typically observed in the general neurotoxin-based animal models; moreover, the transgenic mouse models do not present the selective degeneration of mDA neurons, which is commonly observed in human PD patient. Recent advance in human induced pluripotent stem cell (hiPSC) technology, which can reprogram somatic cells into PSCs, makes it possible to acquire mDA neurons of PD patients.

However, there is the difficulty to model late-onset human disease including PD where patients do not show phenotypes until late in life since the reprogramming somatic cells to iPSCs also reset their pathological state back to an embryonic condition; implicating that accumulated aberrant protein aggregation is a necessary component for modeling disease progression. Several iPSC studies have shown that the differentiation of iPSCs into certain mature cell types often takes months to exhibit disease-associated features. Therefore, despite the emerging use of PD hiPSC-derived mDA neurons, it is still challenging to observe characteristic pathological changes such as the formation of α-syn aggregates. There have been various trials to develop the cellular model using patient-derived hiPSCs to study PD, but it is highly challenging to induce the disease-associated α-syn aggregation in human neurons and most non-cell-based compound screening have a limitation of low reproducibility in human neurons. The α-syn aggregates have been verified by various antibodies that show selectivity for pathological α-syn species over normal monomers or oligomers for the study of PD. Phosphorylation of α-syn at the serine 129 residue (pS129) is the most abundant post-translational modification observed in the PD patient's brain, suggesting that pS129 antibodies could detect pathogenic α-syn aggregates. The monoclonal antibody Syn303 is known to be specific for misfolded α-syn species, and its inhibitory effects against the uptake of preformed fibrils (PFFs) and propagation of α-syn pathology have been reported. Importantly, the 5G4 antibody, which binds aggregated α-syn, has been suggested to show high reactivity for disease-associated forms of α-syn in the PD patient's brain with superior comparative immunohistochemical studies. Thioflavin S (ThioS) staining is also a commonly used method for detecting amyloid fibril formation of α-syn aggregates. Although these antibodies and the fluorescent probe have been extensively used for dissecting α-syn aggregation processes and relevant pathology, the temporal order, and gradual changes of α-syn conformational profiles correspond to different stages of PD progression in human neurons are not fully elucidated. Due to the lack of proper neuronal cell model of controlling α-syn aggregation, most of the previous drug compounds screening efforts utilized biochemical assay with the spontaneous aggregation of α-syn monomers in vitro. However, such assays have a limitation of low reproducibility with slow aggregation reaction which is highly sensitive to pH, temperature, agitation, and purities of α-syn monomer proteins. In addition, recent clinical trials for PD continue to fail; leading to a significant socioeconomic burden on our healthcare system and emphasizing the necessity to develop a new α-syn aggregation/pathogenesis model for future drug discovery efforts.

In recent years, various optogenetic proteins have been developed as a controlling tool of diverse biological processes using light. These optogenetic proteins allow light-induced spatiotemporal control of protein interaction including homo-oligomerization. The power to modulate the protein association/aggregation activity dynamically and precisely has been postulated, but this has yet to be applied in clinically relevant mammalian model systems. Presented herein is a light-inducible pathogenic protein aggregation system (optogenetics-assisted method of alpha-synuclein aggregation induction system, OASIS) on both of human neuronal cells and PD hiPSC-derived mDA neurons, useful to establish OASIS-based drug screening platform for the discovery of novel compounds that inhibit α-syn aggregation. OASIS uses optogenetic proteins to allow light-induced spatiotemporal control of protein interactions, and the development of an OASIS-based drug screening platform. Also presented herein is BAG 956 a compound found in the OASIS-based compound screening, and its ability to rescue α-syn cellular toxicity and pathology in α-syn preformed fibrils (PFF) models in mouse primary neurons and in vivo via autophagy-dependent manner. As provided herein, a BAG 956 treatment was found capable of significantly decreasing toxicity of tau aggregate-induced neurodegeneration in vitro and in vivo.

### SUMMARY OF THE INVENTION

The present invention is based on the seminal discovery that an optogenetic α-synuclein fusion protein can be used to identify α-synuclein aggregation inhibitors, such as BAG 956. Such α-synuclein aggregation inhibitors can be administered to subject having a synucleinopathy or symptoms thereof.

In one embodiment, the present invention provides an isolated nucleic acid sequence including: a first nucleic acid sequence encoding an alpha-synuclein (α-syn) protein; a second nucleic acid sequence encoding a light-responsive domain; and a third nucleic acid sequence encoding a protein tag, in operable linkage. In one aspect, the light-responsive domain is a Cry2 PHR or a Cry2clust light-responsive domain. In another aspect, the protein tag is a hemagglutinin (HA) tag or a mCherry tag. In certain aspects, the light-responsive domain is fused at the C-terminus of the α-syn protein.

In another embodiment, the invention provides a vector including any one the nucleic acid sequences described herein. In one aspect, the vector is a plasmid or a viral vector.

In an additional embodiment, the invention provides an isolated mammalian cell including any one of the vectors described herein. In one aspect, the cell is a terminally differentiated neuron, an induced pluripotent stem cell-derived neural progenitor cell (iPSC NPC), or an iPSC-derived midbrain dopaminergic (iPSC-derived mDA) neuron.

In one embodiment, the present invention provides a method of inducing aggregation of an alpha-synuclein (α-syn) protein in a cell including: contacting the cell with one of the vectors described herein; and exposing the cell to blue light illumination. In one aspect, the cell is an iPSC-derived midbrain dopaminergic (iPSC-derived mDA) neuron. In another aspect, exposing the cell to blue light illumination comprises exposing the cell to an acute pulsed blue light stimulation at a certain light intensity, frequency, and duration. In some aspects, the blue light illumination includes illumination at 470 nm or at 488 nm. In various aspects, the light intensity is about 26 μW/mm² to 34 μW/mm². In other aspects, the frequency is 0.17 Hz, 0.25 HZ, 0.5 Hz or 1 Hz. In many aspects, pulsed blue light stimulation includes 0.5 s pulse or 1 s pulse. In other aspects, the duration is between about 1 hour and 7 days. In one aspect, exposing the cell to blue light illumination generates α-syn aggregates. In other aspects, exposing the cell to blue light illumination generates α-syn aggregates in a time and dose-dependent manner. In some aspects, α-syn aggregates are located in a neurite region and/or in a cell body region of the cell. In many aspects, the α-syn aggregates are insoluble aggregates. In various aspects, the α-syn aggregates generate Lewi bodies in the cell. In one aspect, the α-syn aggregates are pathogenic α-syn aggregates. In various aspects, the α-syn aggregates include 5G4⁺, Syn-O2⁺, pS129⁺, Syn303⁺, p62⁺, ThioS⁺ and/or ubiquitin⁺ α-syn aggregates. In some aspects, the α-syn aggregates decrease cell survival.

In another embodiment, the invention provides a method of identifying an α-syn aggregation inhibitor including: (i) contacting a cell with one of the vectors described herein, (ii) contacting the cell with a test compound, (iii) exposing the cell from (ii) to blue light illumination, and measuring an aggregate induction score (AIS) of the test compound in the cell exposed to blue light illumination (blue AIS); and (vi) exposing a cell from (ii) to the dark and measuring an AIS of the test compound in the cell exposed to the dark (dark AIS), wherein an α-syn aggregation inhibitor has a greater blue AIS as compared to a dark AIS. In one aspect, the cell is an iPSC-derived midbrain dopaminergic (iPSC-derived mDA) neuron. In another aspect, Z′ values of the test compound are further measured. In some aspects, measuring Z′ values include calculating the degree of separation between the blue AIS and the dark AIS. In one aspect, an AIS is the ratio of a number of α-syn aggregates over a number of cells. In another aspect, an α-syn aggregation inhibitor inhibits or delays α-syn aggregation. In some aspects, an α-syn aggregation inhibitor has a blue AIS greater than 0.19. In other aspects, an α-syn aggregation inhibitor increases cell survival.

In an additional embodiment, the invention provides a method of inhibiting the formation of Lewi bodies in a cell including contacting the cell with an α-syn aggregation inhibitor identified by one of the methods described herein. In one aspect, the α-syn aggregation inhibitor is selected from Cyclothiazide, BVT 948, CI 976, NE 100 hydrochloride, BX 471, C 021 dihydrochloride. BAG 956, Arcyriatlavin A, Amlexanox, Kartogenin, Neuropathiazol, Napabucasin, Dexamethasone, XD 14, Mycophenolic acid, Cilostazol, Mycophenolate mofetil, Rizatriptan benzoate, or AZD 1480.

In yet another embodiment, the invention provides a method of treating a synucleinopathy in a subject including administering to the subject in need thereof an α-syn aggregation inhibitor identified by one of the methods described herein. In one aspect, the synucleinopathy is selected from the group consisting of Parkinson Disease (PD), dementia with Lewy body (DLB), multiple system atrophy (MSA), neuroaxonal dystrophy, Alzheimer's disease, primary age-related tauopathy (PART), chronic traumatic encephalopathy (CTE), progressive supranuclear palsy (PSP), corticobasal degeneration (CBD), frontotemporal dementia and parkinsonism linked to chromosome 17 (FTDP-17), amyotrophic lateral sclerosis-parkinsonism-dementia (ALS-PDC, Lytico-bodig disease), ganglioglioma, gangliocytoma, meningioangiomatosis, postencephalitic parkinsonism, subacute sclerosing panencephalitis (SSPE), lead encephalopathy, tuberous sclerosis, pantothenate kinase-associated neurodegeneration, and lipofuscinosis.

In one embodiment, the invention provides an optogenetic alpha-synuclein (α-syn) aggregation system including: (i) a LED illuminator; and (ii) a cell comprising a vector comprising a nucleic acid sequence encoding an α-syn fusion protein. In one aspect, the fusion protein comprises in operable linkage an α-syn protein, a light-responsive domain, and a protein tag. In another aspect, the LED illuminator is a 12-channel, 24-channel, or 96-channel LED illuminator. In some aspects, the system further includes a LED excitation remote controller and a cell culture incubator.

In another embodiment, the invention provides a method of providing neuroprotective effects against a neurodegenerative disease in a subject including, administering to the subject BAG 956, thereby providing neuroprotective effects.

In one aspect, providing neuroprotective effects includes inducing the clearance of alpha-synuclein (α-syn) aggregates, inducing the clearance of tau aggregates, and/or inducing autophagic flux and autophagic degradation of α-syn and/or tau aggregates. In some aspects, inducing the clearance of alpha-synuclein (α-syn) aggregates or inducing the clearance of tau aggregates includes decreasing levels of insoluble pS129−, α-syn, pTau 202/205, pTau 231, pTau 217 and or AT8+ pTau in the subject. In other aspects, inducing autophagic flux and autophagic degradation of α-syn and/or tau aggregates includes inhibiting Pl3K/PDK1/AKT/mTor pathway in dopaminergic neurons, inducing LC3II expression, inhibiting p62 expression, and/or increasing LC3+ and/or LC35G4+ vesicles in dopaminergic neurons. In another aspect, providing neuroprotective effects includes improving grip strength, locomotion and/or hippocampal/amygdala dependent learning and memory in the subject. In one aspect, providing neuroprotective effects includes inhibiting loss of TH+ neurons in the substantia negra. In another aspect, administering comprises oral administration of BAG 956. In some aspects, oral administration of BAG 956 includes oral administration of about 1-20 mg/kg. In various aspects, oral administration of BAG 956 includes about 2 mg/kg or about 10 mg/kg. In one aspect, the neurodegenerative disease is selected from the group consisting of dementia with Lewy body (DLB), multiple system atrophy (MSA), neuroaxonal dystrophy, Alzheimer's disease, primary age-related tauopathy (PART), chronic traumatic encephalopathy (CTE), progressive supranuclear palsy (PSP), corticobasal degeneration (CBD), frontotemporal dementia and parkinsonism linked to chromosome 17 (FTDP-17), amyotrophic lateral sclerosis-parkinsonism-dementia (ALS-PDC, Lytico-bodig disease), ganglioglioma, gangliocytoma, meningioangiomatosis, postencephalitic parkinsonism, subacute sclerosing panencephalitis (SSPE), lead encephalopathy, tuberous sclerosis, pantothenate kinase-associated neurodegeneration, lipofuscinosis, and Parkinson's disease. In various aspect, the neurodegenerative disease is Parkinson's disease.

### BRIEF DESCRIPTION OF THE DRAWINGS

FIG. 1 illustrates the alpha-synuclein aggregation system (OASIS) principle.

FIGS. 2A-2F illustrate light-induced aggregation of α-syn. FIG. 2A is a schematic representation of the opto-aggregation system used to accelerate and precisely control the formation of disease-associated α-syn aggregate. FIG. 2B illustrates the quantification of the percentage of aggregate⁺ cells, relative to the number of transfected cells. FIG. 2C illustrates the quantification of the percentage of phosphorylated-α-syn⁺ (p-α-syn⁺) cells, relative to the number of transfected cells. FIG. 2D illustrates a schematic for AAVS1 locus targeting using homologous recombination enhanced by CRISPR/Cas9 system. SA, splice acceptor. FIG. 2E is a graph illustrating the quantification of the percentage of aggregate⁺ cells, relative to the number of DAPl⁺ cells. FIG. 2F is a graph illustrating the quantification of the aggregated-α-syn. Error bars represent mean±SEM. n.s., not significant. *P<0.05, **P<0.01, ***P<0.001, ****P<0.0001.

FIGS. 3A-3C illustrate customized blue light illuminating plates in a CO2 incubator and the expression of HA-opto-mock or HA-opto-α-syn SH-SY5Y neuronal cells. FIG. 3A shows representatives images of a customized blue light illumination plate in a cell culture incubator. FIG. 3B shows a immunoblot analysis with anti-HA antibody. FIG. 3C shows scatter plots illustrating global transcriptome analyses (RNA-seq) of the indicated conditions. Scatter plots of all expressed genes in each pairwise (dots, P<0.05 with Benjamini-Hochberg multiple testing correction).

FIGS. 4A-4B illustrate the generation of AAVS1::opto-mok or AAVS1::opto-α-syn PD hiPSCs. FIG. 4A illustrates schematic representation of the various protein constructs including opto-mock, mCherry-α-syn, N-opto-α-syn, and C-opto-α-syn. Opto-mock, N-opto-α-syn, and C-opto-α-syn have Cry2Clst domain for blue light-induced protein interaction. FIG. 4B illustrates an electrophoresis gel of Genomic DNA PCR of AAVS1::opto-mock or AAVS1::opto-α-syn PD hiPSCs.

FIGS. 5A-5F illustrate light-induced disease-associated α-syn aggregation. FIG. 5A illustrate schematic of AAVS1 locus targeted using homologous recombination enhanced by CRISPR/Cas9 system in PD hiPSCs. FIG. 5B PD hiPSCs differentiation into mDA neurons. FIGURE 5C is a graph illustrating the total area of aggregate in mDA neurons expressing opto-mock or opto-α-syn in cell body. FIG. 5D is a graph illustrating in neurite of opto-α-syn-expressing mDA neurons. FIG. 5E is a graph illustrating the number of 5G4⁺ aggregates in opto-mock- or opto-α-syn-expressing mDA neurons with or without blue light illumination. FIG. 5F is a graph illustrating the number of pS129′ α-syn aggregates in opto-mock- or opto-α-syn-expressing mDA neurons with or without blue light illumination. Error bars represent mean±SD. Error bars represent mean±SEM. n.s., not significant. ****P<0.0001.

FIGS. 6A-6B illustrate neural differentiation onto TH+ mDA neurons from AAVS1::opto-mock or AAVS1::opto-α-syn PD hiPSCs. FIG. 6A is a graph bar showing the quantification of TH⁺ mDA neurons expressing opto-mock or opto-α-syn. Error bars represent mean±SD. FIG. 6B is a graph illustrating the quantification of relative levels of cell number to control.

FIGS. 7A-7C show the selective death of PD hiPSC-derived mDA neurons induced by the optogenetic α-syn aggregation system. FIG. 7A is a graph illustrating the number of aggregates in Opto-α-syn-mDA neurons immunostained with Syn303, EP1536Y. and 5G4 or stained with ThioS. Error bars represent mean±SEM. FIG. 7B is a graph illustrating the quantification of the TUJ1⁺ area per DAPI. Error bars represent mean±SEM. FIG. 7C is a graph illustrating the quantification of the TH⁺ area normalized to DAPI (H). Error bars represent mean±SD. Error bars represent mean±SEM. n.s., not significant. **P<0.01.

FIGS. 8A-8E illustrate high-content imaging screening with the optogenetic α-syn aggregation system. FIG. 8A is a schematic representation of the process of high-content imaging (HCI) screening with optogenetics-assisted method of alpha-synuclein aggregation induction system (OASIS). FIG. 8B shows the equation of Aggregates Induction Score (AIS). FIG. 8C is a graph illustrating the calculation of Z′-factor for HCI screening with OASIS. Dots represent wells with the following treatment: opto-α-syn cells in dark (upper circles) or exposed to blue light (lower circles). Arrow represents the degree of separation (Z′-factor) between light-illuminated and darkness controls. FIGURE 8D is a scatter plot of compounds screened in the OASIS-based HCI assay. FIG. 8E is a graph bar illustrating validating effect of treatment with 19 compounds on α-syn aggregation in HA-opto-α-syn SH-SY5Y neuronal *P<0.05, **P<0.01, ***P<0.001, ****P<0.0001.

FIGS. 9A-9D illustrate high-content imaging screening with the optogenetic α-syn aggregation system. FIG. 9A illustrates the measurement of 5G4⁺ aggregates in opto-mock or opto-α-syn SH-SY5Y neuronal cells. FIG. 9B illustrates the counting the number of DAPI from the original images. FIG. 9C shows a flow chart schematically representing the main steps of SEA-mediated target analysis. FIG. 9D is a graph illustrating the combined Z score of target proteins obtained from 19 compounds screened through OASIS.

FIGS. 10A-10C illustrate the validation of potential hit compounds from primary screening through comparative chemogenomic analysis with PD clinical drugs. FIG. 10A shows drug-protein interaction matrix for the significantly enriched 89 drug target proteins from 19 compounds screened by OASIS. FIG. 10B shows drug-protein interaction matrix for the significantly enriched 85 drug target proteins from 17 PD clinical drugs. Shading represents the significance of the predicted interaction based on its z-score. FIG. 10C is a graph showing a comparative Gene Ontology (GO) term dot plots from significant target proteins of CRC clinical drugs, compounds screened by OASIS, and PD clinical drugs. Y-axis indicates the GO term and X-axis shows the GeneRatio per GO term. Gradient and size of dots represent adjusted p-values and GeneRatio, respectively.

FIGS. 11A-11F illustrate the confirmation of the effects of 5 selected compounds on the light-induced α-syn aggregation in PD hiPSC-derived mDA neurons. FIG. 11A is a graph bar illustrating the quantification of the aggregated-α-syn⁺ area per DAPI. FIG. 11B is a graph bar illustrating the quantification of the aggregated TH⁺ area per DAPI. FIG. 11C is a graph bar illustrating the quantification of the aggregated TUJ1⁺ area per DAPI. FIG. 11D is a flow chart illustrating that 2 out of a total of 1,280 chemicals were screened by high-content imaging-mediated optogenetics-assisted method of alpha-synuclein aggregation induction system (OASIS). FIG. 11E is a bar graph of Gene Ontology (GO) enrichment analysis. FIG. 11F illustrates heat map displays log 2 fold change values of the expression of selected differentially expressed genes related with GO terms. *P<0.05, **P<0.01, ***P<0.001, ****P<0.0001.

FIGS. 12A-12C illustrate the confirmation of the BAG 956 effect on α-syn PFF treated neurons in vitro. FIG. 12A is an immunoblot of pS129-α-syn in TX-100 insoluble fraction. FIG. 12B is a graph showing the quantification of pS129-α-syn in TX-100 insoluble fraction. FIG. 12C is a graph illustrating the quantification of pS129-α-syn in PD hiPSC-derived mDA neurons treated with α-syn PFF with vehicle, BAG 956, or CDC 021 for 14 days. Bars represent means±SEM. One-way ANOVA followed by Tukey's post hoc test. n.s., not significant, *P<0.05, **P<0.01, ***P<0.001, ****P<0.0001.

FIGS. 13A-13L illustrate the behavioral deficits and PD-like pathologies induced by α-syn PFF injection recovered by oral administration of BAG 956. FIG. 13A is a schematical diagram for behavioral assessment at 7 months after intrastriatal injection of PBS or α-syn PFF (n=8). FIG. 13B is a graph illustrating fore- and hind-limb grip strength test. FIG. 13C is a graph showing latency to fall from the rotarod test. FIG. 13D shows representative locomotion and central activity of each group via travelled path in the OFT. FIG. 13E shows the number of entries (left), time spent (middle), and distance travelled (right) in the center zone of the OFT. FIG. 13F shows representative movement paths of mice from each group in the EPM. FIG. 13G shows the number of entries (left) and time spent (right) in the open area far from center zone. FIG. 13H shows the evaluation α-syn PFF induced loss of learning and memory by total freeze time and freezing episode from cued FC. FIG. 13I shows representative photomicrographs from coronal mesencephalon sections including TH-positive and Nissl-positive neurons in PBS or α-syn PFF with or without BAG 956. FIG. 13J shows quantification of representative photomicrographs from coronal mesencephalon sections including TH-positive and Nissl-positive neurons in PBS or α-syn PFF with or without BAG 956. FIG. 13K shows representative immunoblot from insoluble (upper) and soluble (lower) fraction of ventral midbrain regions. FIG. 13L shows quantification (M) of pS129-α-syn protein level (upper) and TH level (lower). Bars represent means±SEM. One-way ANOVA followed by Tukey's post hoc test. *P<0.05, **P<0.01, ***P<0.001, ****P<0.0001. n.s., not significant. Scale bars, 100 μm.

FIGS. 14A-14I illustrate the induction of autophagy-mediated clearance of α-syn-aggregates through inhibition of PI3K-PDK1/AKT/mTOR pathway by BAG 956 treatment. FIG. 14A shows representative western blot images. FIG. 14B shows the quantification of LC3-II and β-actin in PD hiPSC-derived mDA neurons treated with PBS or α-syn PFF with vehicle, BAG 956, or CDC 021. FIG. 14C shows the quantification of LC3-I, p62 and β-actin in PD hiPSC-derived mDA neurons treated with PBS or α-syn PFF with vehicle, BAG 956, or CDC 021. FIG. 14D shows and the quantification of LC3⁺/5G4⁺ co-localized dots in opto-α-syn-MOs kept in dark after blue light illumination (6 days). FIG. 14E shows and the quantification of 5G4⁺ α-syn aggregates in opto-α-syn-mDA neurons with vehicle, BAG 956, or BAG 956 and Bafilomycin A1 (Bafilo). FIG. 14F shows the quantification of TH⁺ neurons in opto-α-syn-mDA neurons with vehicle, BAG 956, or BAG 956 and Bafilomycin A1 (Bafilo). FIG. 14G shows representative western blot images of PI3K/PDK1 downstream targets in PD hiPSC-derived mDA neurons treated with PBS or α-syn PFF with vehicle or BAG 956. FIG. 14H shows representative western blot images of PI3K/PDK1 downstream targets in mouse ventral midbrain treated with PBS or α-syn PFF with vehicle or BAG 956. FIG. 14I shows the quantification of pS473-AKT normalized to pan-AKT in FIG. 14H. Bars represent means±SEM. One-way ANOVA followed by Tukey's post hoc test. n.s., not significant. *P<0.05, **P<0.01, ***P<0.001, ****P<0.0001. Blue light condition: 34 μW/mm² at 470 nm, 0.17 Hz, 0.5 s, for 7 days.

FIGS. 15A-15D illustrate how BAG treatment significantly reduces the p-TAU levels caused by tau PFF treatment in mouse cortical neurons. FIG. 15A shows representative western blot images of P-tau levels as detected with AT8, AT180, and Tau-217. FIG. 15B shows the quantification of AT8. FIG. 15C shows the quantification of AT180. FIG. 15D shows the quantification of Tau-217. Data are the means±SEM, PBS group, n=4 independent experiments, tau-PFF groups, n=4 independent experiments, one-way ANOVA followed by Tukey's correction; *P<0.05, **P<0.01, ***P<0.001, ****P<0.0001.

FIGS. 16A-16B show the quantification and statistical analysis results of the area covered by AT8 (%) of two locations in mouse brain samples. FIG. 16A shows the quantification and statistical analysis results of the area covered by AT8 (%) in location A. FIG. 16B shows the quantification and statistical analysis results of the area covered by AT8 (%) in location B, respectively. One-way ANOVA followed by Tukey's post hoc test. ns, not significant, ****P<0.0001.

FIGS. 17A-17H illustrate the neuroprotective effects of BAG 956 treatment on α-syn PFF induced pathology. FIG. 17A is a schematic diagram of 3 months immunofluorescence assessment after α-syn PFF injection. FIG. 17B shows the quantification from coronal mesencephalon sections containing TH- and pS129-α-syn-positive immunoreactivities in PBS, PBS^{High}, PFF, PFF^{Low}, and PFF^{High} respectively. FIG. 17C shows the end point body mass differences in each group are shown. FIG. 17D shows the behavioral assessment at 7 months after α-syn PFF injection evaluated from EPM with activities of total open arm area. FIG. 17E shows the behavioral assessment at 7 months after α-syn PFF injection evaluated from EPM with activities of near to center zone. FIG. 17F shows FC from contextual test. FIG. 17G shows representative photomicrograph of ipsilateral striatal sections stained to testify TH immunoreactivity. FIG. 17H shows TH protein levels in ipsilateral striatum region of each group. Scale bars, 100 μm. Error bars represent the mean±SEM. Statistical significance was determined by a one-way ANOVA followed by Tukey's multiple comparisons test. *P<0.05, **P<0.01, ***P<0.001, ****P<0.0001. n.s., not significant.

FIGS. 18A-18I illustrate BAG 956-selective neuroprotection effects in autophagy-dependent manner through PI3K-AKT-mTOR signaling. FIG. 18A shows representative western blot images of LC3 and β-actin in opto-α-syn mDA neurons kept in dark or exposed to blue light with or without BAG 956. FIG. 18B shows the quantification of LC3-II in FIG. 16A. FIG. 18C shows representative western blot images of LC3 and β-actin in opto-α-syn mDA neurons kept in dark or exposed to blue light with vehicle, BAG 956, BYL719 (BLY), or BAY 80-6946 (BAY). FIG. 18D shows the quantification of LC3⁺ puncta in opto-α-syn-expressing MOs kept in dark after blue light illumination (6 days). FIG. 18E shows the quantification of LC3⁺ puncta in α-syn mDA neurons exposed to blue light with vehicle, CDC 021, or BAG 956 (n=16). FIG. 18F shows the quantification of LC3⁺/5G4⁺ co-localized vesicles in α-syn mDA neurons exposed to blue light with vehicle, CDC 021, or BAG 956 (n=16). FIG. 18G shows representative western blot images of PI3K/PDK1 downstream targets (p-mTOR, p-AKT, p-S6K) in Triton X-100 soluble fraction or pS129-α-syn, α-syn, P62, LC3 and β-actin in Triton X-100 insoluble fraction from mouse primary neurons treated with PBS or PFF with vehicle, BAG 956. FIG. 18H shows the quantification of p-mTOR, pS308-AKT normalized to each pan-proteins in mouse ventral midbrain treated with PBS or PFF with vehicle, BAG 956 2 mg/kg or 10 mg/kg. FIG. 18I shows a schematic representation of the BAG 956 action of mechanism in autophagy enhancement through PI3K/AKT/mTOR pathway. Blue light condition: 34 μW/mm² at 470 nm, 0.17 Hz, 0.5 s, for 7 days.

### DETAILED DESCRIPTION OF THE INVENTION

The present invention is based on the seminal discovery that an optogenetic α-synuclein fusion protein can be used to identify α-synuclein aggregation inhibitors. Such α-synuclein aggregation inhibitors can be administered to subject having a synucleinopathy or symptoms thereof.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides an isolated nucleic acid sequence including: a first nucleic acid sequence encoding an alpha-synuclein (α-syn) protein; a second nucleic acid sequence encoding a light-responsive domain; and a third nucleic acid sequence encoding a protein tag, in operable linkage.

As used herein, the phrase “nucleic acid” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic can be employed for introduction into (i.e., transfection of) cells, in particular, in the form of RNA which can be prepared by in vitro transcription from a DNA template. The RNA can moreover be modified before application by stabilizing sequences, capping, and polyadenylation.

The nucleic acid may be extracted from a sample, by any method known in the art including by using organic solvents such as a mixture of phenol and chloroform, followed by precipitation with ethanol. Among other methods of extracting cell-free nucleic acid, one such method includes, for example, using polylysine-coated silica particles. Alternatively, the nucleic acid may be extracted using commercially available kit such as, for example, QIAamp® DNA minikit (Qiagen, Germantown, MD).

The extracted nucleic acid can be amplified by one of the alternative methods for amplification well known in the art, which include for example: the Xmap® technology of Luminex that allows the simultaneous analysis of up to 500 bioassays through the reading of biological test on the surface of microscopic polystyrene bead; the multiplex PCR that allows the simultaneous amplification of several DNA sequences; the multiplex ligation-dependent probe amplification (MLPA) for the amplification of multiple targets using a single pair of primers; the quantitative PCR (qPCR), which measures and quantify the amplification in real time; the ligation chain reaction (LCR) that uses primers covering the entire sequence to amplify, thereby preventing the amplification of sequences with a mutation; the rolling circle amplification (RCA), wherein the two ends of the sequences are joined by a ligase prior to the amplification of the circular DNA; the helicase dependent amplification (HDA) which relies on a helicase for the separation of the double stranded DNA; the loop mediated isothermal amplification (LAMP) which employs a DNA polymerase with high strand displacement activity; the nucleic acid sequence based amplification, specifically designed for RNA targets; the strand displacement amplification (SDA) which relies on a strand-displacing DNA polymerase, to initiate replication at nicks created by a strand-limited restriction endonuclease or nicking enzyme at a site contained in a primer, and the multiple displacement amplification (MDA), based on the use of the highly processive and strand displacing DNA polymerase from the bacteriophage Ø29. amplification methods as used herein have been used and tested, and are well known in the art.

As used herein “amplified DNA” or “PCR product” refers to an amplified fragment of DNA of defined size. Various techniques are available and well known in the art to detect PCR products. PCR product detection methods include, but are not restricted to, gel electrophoresis using agarose or polyacrylamide gel and adding ethidium bromide staining (a DNA intercalant), labeled probes (radioactive or non-radioactive labels, southern blotting), labeled deoxyribonucleotides (for the direct incorporation of radioactive or non-radioactive labels) or silver staining for the direct visualization of the amplified PCR products; restriction endonuclease digestion, that relies agarose or polyacrylamide gel or High-performance liquid chromatography (HPLC); dot blots, using the hybridization of the amplified DNA on specific labeled probes (radioactive or non-radioactive labels); high-pressure liquid chromatography using ultraviolet detection; electro-chemiluminescence coupled with voltage-initiated chemical reaction/photon detection; and direct sequencing using radioactive or fluorescently labeled deoxyribonucleotides for the determination of the precise order of nucleotides with a DNA fragment of interest, oligo ligation assay (OLA), PCR, qPCR, DNA sequencing, fluorescence, gel electrophoresis, magnetic beads, allele specific primer extension (ASPE) and/or direct hybridization. Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; U.S. Pub. 2010/0285578; and U.S. Pub. 200210190663.

Nucleic acid can be analyzed in various ways, include, but not limited to, sequencing and DNA-protein interaction. Sequencing may be by any method known in the art. DNA sequencing techniques include classic dideoxy sequencing reactions (Sanger method) using labeled terminators or primers and gel separation in slab or capillary, and next generation sequencing methods such as sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

The nucleic acid sequence can be a “protein coding sequence” or a sequence that encodes a particular polypeptide or peptide. Such nucleic acid sequence is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence. The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof.

The nucleic acid sequence can encode an alpha-synuclein (α-syn) protein. Alpha-synuclein is a protein that, in humans, is encoded by the SNCA gene (accession numbers NM 000345.3 and NP 000336.1). α-syn is abundant in the brain (predominantly expressed in the neocortex, hippocampus, substantia nigra, thalamus, and cerebellum), and mainly expressed at presynaptic terminals of neurons where it interacts with phospholipids and proteins. At least three isoforms of synuclein are produced through alternative splicing, but the mainly expressed form of the protein is the full-length protein of 140 amino acids, which includes three distinct domains. Residues 1-60 encode an amphipathic N-terminal region dominated by four 11-residue repeats including the consensus sequence KTKEGV (SEQ ID NO:1) having a structural alpha helix propensity similar to apolipoproteins-binding domains. It is a highly conserved terminal that interacts with acidic lipid membranes, and all the discovered point mutations of the SNCA gene are located within this terminal. Residues 61-95 encode a central hydrophobic region which includes the non-amyloid-β component (NAC) region, involved in protein aggregation. This domain is unique to alpha-synuclein among the synuclein family. Residues 96-140 encode a highly acidic and proline-rich region which has no distinct structural propensity. This domain plays an important role in the function, solubility, and interaction of alpha-synuclein with other proteins.

Unmutated α-synuclein forms a stably folded tetramer that resists aggregation, however, in pathological conditions. α-syn can aggregate and form insoluble fibrils. The aggregation mechanism of alpha-synuclein is uncertain and might rely on structured intermediate rich in beta structure that can be the precursor of aggregation and, ultimately, Lewy bodies. Unfolded monomer can aggregate first into small oligomeric species that can be stabilized by β-sheet-like interactions and then into higher molecular weight insoluble fibrils. Protein modifications such as phosphorylation (such as phosphorylation at Ser129 by polo-like kinase 2 (PLK2) kinase), truncation (through proteases such as calpains), and nitration (probably through nitric oxide (NO) or other reactive nitrogen species that are present during inflammation), modify synuclein such that it has a higher tendency to aggregate. The addition of ubiquitin to Lewy bodies is a secondary process to deposition.

Genetic alterations of the SNCA gene, can also result in aberrant polymerization of α-syn into insoluble fibrils, which are associated with several neurodegenerative diseases (synucleinopathies).

The nucleic acid sequence can encode a light-responsive domain. As used herein, a “light-responsive domain” is a photosensitive protein or protein domain that undergoes a conformational change upon illumination, and consequently, induces protein interaction. Such photosensitive protein can be used in an optogenetic dimerization system comprising two compatible domains that can interact with one another upon illumination. Optogenetic systems can be based on natural photoreceptors that contain a chromophore that undergoes isomerization or formation of a chemical bond upon absorption of a photon, leading to a conformational change in the photoreceptor that is eventually propagated to the effector domain. Although some photoreceptors, such as rhodopsin, integrate both sensory and effector functions, most photoreceptors, such as light-oxygen-voltage (LOV) proteins, cryptochromes (CRYs), and phytochromes, mediate intra- or intermolecular interactions in response to light.

LOV domains are flavin mononucleotide (FMN) binding photosensors and form a transient covalent bond to FMN molecules upon blue-light activation that may remain stable for seconds to days. Examples of LOV domains include the LOV2 domain from Avena sativa phototropin, which can interact with various protein or peptide.

CRY proteins are photoreceptors that contain a conserved N-terminal photolyase homology region (PHR) that binds a flavin adenine dinucleotide (FAD) chromophore. A light-induced dimerization system was developed based on the CRY2 domain from A. thaliana, which bound CRY-interacting basic-helix-loop-helix (CIB1) or its shorter N-terminal variant (CIBN) in its photoexcited state. The light-induced dimerization of CRY2 with CIBN is complete within 10 s and slowly reverses over 12 min in the dark. New engineered variants of CRY2 have been developed to improve the dynamic range (reduced dark activity) and to alter photocycle kinetics with longer or shorter half-lives for CIB1 binding.

Other photosensitive proteins with absorption at different wavelengths, such as UVR8: the fluorescent protein (FP) Dronpa; and cobalamin (vitamin B12) binding domains (CBDs) have been added to the optogenetic toolbox.

Non limiting examples of optogenetic dimerization systems include UVR8-COP1, UVR8-UVR8, FKFI-GI, TULIPs, LOVpep-ePDZ, iLID, LOVSsrA-SsrB, LightOn, VVD-VVD, Magnets, pMag-nMag (VVD variants), LOVTRAP, LOV2-Zdk, CRY2-CIB1/CIBN, CRY2-CIB1 variants. CRY2-CRY2, CRY2 olig, CRY2-CRY2 (E490G mutant), Dronpa-Dronpa, CBD-CBD, PhyB-PIF3/6, Cph1-Cph1, BphP1-PpsR2, and any variants thereof.

In one aspect, the optogenetic dimerization systems is a CRY2-CRY2 system comprising two CRY2 light-responsive domains. In one aspect, the light-responsive domain is a Cry2 PHR or a Cry2clust light-responsive domain.

The nucleic acid sequence can encode protein tag. A variety of protein tags are known in the art, such as epitope tags, affinity tags, fluorescent tags, solubility enhancing tags, and the like. Affinity tags are the most commonly used tag for aiding in protein purification while epitope tags aid in the identification of proteins. Epitope tags are short peptide sequences which are chosen because high-affinity antibodies can be reliably produced in many different species. These are usually derived from viral genes, which explain their high immunoreactivity. Epitope tags include ALFA-tag, V5-tag, Myc-tag, HA-tag, Spot-tag, T7-tag, and NE-tag. These tags are particularly useful for western blotting, immunofluorescence, and immunoprecipitation experiments, although they also find use in antibody purification. Fluorescence tags are used to give visual readout on a protein. GFP and its variants are the most commonly used fluorescence tags, but any known fluorescent tag can be used. As used herein, the term “protein tag” refers to any protein or protein domain that can be used to detect, purify, or quantify the α-syn protein. In one aspect, the protein tag is a hemagglutinin (HA) tag or a mCherry tag.

The nucleic acid sequences are in operable linkage with one another, such that the resulting encoded polypeptide is a biologically active fusion protein. As used herein the terms “fusion molecule” and “fusion protein” are used interchangeably and are meant to refer to a biologically active polypeptide, where the independent protein or protein domain of the fusion protein (the α-syn protein, the protein tag, and the light-responsive domain) are covalently linked (i.e., fused) by recombinant, chemical or other suitable method. If desired, the fusion molecule can be used at one or several sites through a peptide linker sequence. Alternatively, the peptide linker may be used to assist in construction of the fusion molecule. Specifically, preferred fusion molecules are fusion proteins. Generally, fusion molecule also can include conjugate molecules. The fusion protein of the present invention is a fusion protein of an α-syn, a protein tag, and a light-responsive domain. It can be referred to as an “opto-α-syn protein”, an “opto-α-syn fusion protein”, an “optogenetic α-syn protein”, an “optogenetic α-syn fusion protein” and the like without any difference in meaning.

The sequences encoding the α-syn protein, the protein tag and the light-responsive domain can be operatively linked to one another in any order. For example, the α-syn protein can be at the C-terminus of the fusion protein, at the N-terminus of the fusion protein, or in between the protein tag, and the light-responsive domain; the protein tag can be at the C-terminus of the fusion protein, at the N-terminus of the fusion protein, or in between the α-syn, and the light-responsive domain; the light-responsive domain can be at the C-terminus of the fusion protein, at the N-terminus of the fusion protein, or in between the protein tag, and the α-syn. In certain aspects, the light-responsive domain is fused at the C-terminus of the α-syn protein.

An isolated nucleic acid sequence including a nucleic acid sequence encoding an alpha-synuclein (α-syn) protein, a nucleic acid sequence encoding a light-responsive domain and a nucleic acid sequence encoding a protein tag, in operable linkage can be incorporated into an expression cassette (e.g., a circular or linear polynucleotide including one or more genes or interest operably linked to one or more regulatory sequences) to be delivered to a cell in a vector. A vector can be an integrating or non-integrating vector, referring to the ability of the vector to integrate the expression cassette into a genome of a cell. Integrating vector and non-integrating vector can be used to deliver an expression cassette containing a gene operably linked to a regulatory element into a cell, to induce the expression of the recombinant nucleic acid construct. Regulatory elements can include promoter, protein tags, functional domains, regulatory sequences, and the like. Examples of vectors include, but are not limited to, (a) non-viral vectors such as nucleic acid vectors including linear oligonucleotides and circular plasmids; and (b) viral vectors such as retroviral vectors, lentiviral vectors, adenoviral vectors, and AAV vectors. Viruses have several advantages for delivery of nucleic acids, including high infectivity and/or tropism for certain target cells or tissues.

Vectors suitable for use in preparation of proteins and/or protein conjugates include those selected from baculovirus, phage, plasmid, phagemid, cosmid, fosmid, bacterial artificial chromosome, viral DNA, PI-based artificial chromosome, yeast plasmid, and yeast artificial chromosome. For example, the viral DNA vector can be selected from vaccinia, adenovirus, foul pox virus, pseudorabies, and a derivative of SV40.

Suitable bacterial vectors for use in practice of the invention methods include pQE70™, pQE60™, pQE-9™, pBLUESCRIPT™ SK, pBLUESCRIPT™ KS, pTRC99a™, pKK223-3™, pDR540™, PACT™ and pRIT2T™. Suitable eukaryotic vectors for use in practice of the invention methods include pWLNEO™, pXTI™, pSG5™, pSVK3™, pBPV™, pMSG™, and pSVLSV4™. Suitable eukaryotic vectors for use in practice of the invention methods include pWLNEO™, pXTI™, pSG5™, pSVK3™, pBPV™, pMSG™, and pSVLSV40™. One type of vector is a genomic integrated vector, or “integrated vector,” which can become integrated into the chromosomal DNA of the host cell. Another type of vector is an episomal vector, e.g., a nucleic acid capable of extra-chromosomal replication. Vectors capable of directing the expression of genes to which they are operatively linked are referred to herein as “expression vectors.”

Suitable viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so the native tropism and pathogenicity of the virus has been altered or removed. The genome of a virus also can be modified to increase its infectivity and to accommodate packaging of the nucleic acid encoding the polypeptide of interest. The term “AAV” covers all serotypes, subtypes, and both naturally occurring and recombinant forms, except where required otherwise. The abbreviation “rAAV” refers to recombinant adeno-associated virus, also referred to as a recombinant AAV vector (or “rAAV vector”). Suitable AAV vectors include AAV1, AAV2, AAV3, AAV4, AAV5, AAV6, AAV7, AAV8, AAV9, AAV10, AAV11, AAV 12, rhlO, and hybrids thereof, avian AAV, bovine AAV, canine AAV, equine AAV, primate AAV, non-primate AAV, and ovine AAV. The use of “lentiviral vector” in gene therapy refers to a method by which genes can be inserted, modified, or deleted in organisms using lentivirus. Lentiviruses are a family of viruses which infect by inserting DNA into their host cells' genome. Many such viruses have been the basis of research using viruses in gene therapy, but the lentivirus is unique in its ability to infect non-dividing cells, and therefore has a wider range of potential applications. Lentiviruses can become endogenous (ERV), integrating their genome into the host germline genome, so that the virus is henceforth inherited by the host's descendants. To be effective in gene therapy, there must be insertion, alteration and/or removal of host cell genes. To do this, scientists use the lentivirus' mechanisms of infection to achieve a desired outcome to gene therapy. Non-limiting examples or lentivirus that can be used for gene therapy include those derived from bovine immunodeficiency virus, caprine arthritis encephalitis virus, equine infectious anemia virus, feline immunodeficiency virus, Human immunodeficiency virus 1, Human immunodeficiency virus 2, Jembrana disease virus, puma lentivirus, simian immunodeficiency virus or Visna-maedi virus.

Regulatory elements controlling transcription can be generally derived from mammalian, microbial, viral or insect genes. Non-limiting examples of regulatory elements include promoter, polyadenylation sequences, translation control sequences (e.g., an internal ribosome entry segment, IRES), enhancers, or introns. Such elements may not be necessary, although they may increase expression by affecting transcription, stability of the mRNA, translational efficiency, or the like. Such elements can be included in a nucleic acid construct as desired to obtain optimal expression of the nucleic acids in the cell(s). For example, a vector usually comprises one or more promoters, operably linked to the nucleic acid of interest, used to facilitate transcription of genes in operable linkage with the promoter. Several types of promoters are well known in the art and suitable for use with the present invention. The promoter can be constitutive or inducible. Non-limiting examples of constitutive promoters include cytomegalovirus (CMV) promoter and the Rous sarcoma virus promoter, that allows for unregulated expression in mammalian cells. A vector can include a nucleic acid that encodes a signal peptide such that the encoded polypeptide is directed to a particular cellular location (e.g., a signal secretion sequence to cause the protein to be secreted by the cell) or a nucleic acid that encodes a selectable marker to facilitate recognition of transformants. Non-limiting examples of selectable markers include puromycin, adenosine deaminase (ADA), aminoglycoside phosphotransferase (neo, G418, APH), dihydrofolate reductase (DHFR), hygromycin-B-phosphotransferase, thymidine kinase (TK), and xanthin-guanine phosphoribosyl transferase (XGPRT). Such markers are useful for selecting stable transformants in culture. The ability to replicate in a host can also be conferred to a vector by incorporating an origin of replication. Those of skill in the art can select a suitable regulatory region to be included in such a vector.

In an embodiment, the invention provides a vector including any one the nucleic acid sequences described herein. In one aspect, the vector is a plasmid or a viral vector.

A vector including isolated nucleic acid sequence including a nucleic acid sequence encoding an alpha-synuclein (α-syn) protein, a nucleic acid sequence encoding a light-responsive domain and a nucleic acid sequence encoding a protein tag, in operable linkage can be delivered to a host cell to be altered thus allowing expression of the fusion protein within the cell. A variety of host cells are known in the art and suitable for chimeric proteins expression. Examples of typical cell used for transfection include, but are not limited to, a bacterial cell, a eukaryotic cell, a yeast cell, an insect cell, or a plant cell.

In an additional embodiment, the invention provides an isolated mammalian cell including any one of the vectors described herein. In one aspect, the cell is a terminally differentiated neuron, an induced pluripotent stem cell-derived neural progenitor cell (iPSC NPC), or an iPSC-derived midbrain dopaminergic (iPSC-derived mDA) neuron.

Induced pluripotent stem cells, “iPS cells” or “iPSCs” are a type of pluripotent stem cell that can be generated directly from a somatic cell. Because they can propagate indefinitely, as well as give rise to every other cell type in the body (such as neurons, heart, pancreatic, and liver cells), they represent a single source of cells that could be used to replace those lost to damage or disease. The most well-known type of pluripotent stem cell is the embryonic stem cell. However, since the generation of embryonic stem cells involves destruction (or at least manipulation) of the pre-implantation stage embryo, there has been much controversy surrounding their use.

Since iPSCs can be derived directly from adult tissues, they not only bypass the need for embryos, but can be made in a patient-matched manner, which means that each individual could have their own pluripotent stem cell line. These unlimited supplies of autologous cells could be used to generate transplants without the risk of immune rejection. iPSCs are typically derived by introducing products of specific sets of pluripotency-associated genes, or “reprogramming factors”, into a given cell type. The original set of reprogramming factors (Yamanaka factors) are the transcription factors Oct4 (Pou5fl), Sox2, Klf4 and cMyc. While this combination is most conventional in producing iPSCs, each of the factors can be functionally replaced by related transcription factors, miRNAs, small molecules, or even non-related genes such as lineage specifiers. It is also clear that pro-mitotic factors such as C-MYC/L-MYC or repression of cell cycle checkpoints, such as p53, are conduits to creating a compliant cellular state for iPSC reprograming. iPSC derivation is typically a slow and inefficient process, taking 1-**2 weeks for mouse cells and 3-4 weeks for human cells, with efficiencies around 0.01-0.1%. However, considerable advances have been made in improving the efficiency and the time it takes to obtain iPSCs. Upon introduction of reprogramming factors, cells begin to form colonies that resemble pluripotent stem cells, which can be isolated based on their morphology, conditions that select for their growth, or through expression of surface markers or reporter genes.

Dopaminergic pathways (dopamine pathways, dopaminergic projections) in the human brain are involved in both physiological and behavioral processes including movement, cognition, executive functions, reward, motivation, and neuroendocrine control. Each pathway is a set of projection neurons, consisting of individual dopaminergic neurons. The four major dopaminergic pathways are the mesolimbic pathway, the mesocortical pathway, the nigrostriatal pathway, and the tuberoinfundibular pathway. The mesolimbic pathway and the mesocortical pathway form the mesocorticolimbic system. Two other dopaminergic pathways to be considered are the hypothalamospinal tract and the incertohypothalamic pathway.

Parkinson's disease, attention deficit hyperactivity disorder (ADHD), substance use disorders (addiction), and restless legs syndrome (RLS) can be attributed to dysfunction in specific dopaminergic pathways.

The dopamine neurons of the dopaminergic pathways synthesize and release the neurotransmitter dopamine. Enzymes tyrosine hydroxylase and dopa decarboxylase are required for dopamine synthesis. These enzymes are both produced in the cell bodies of dopamine neurons. Dopamine is stored in the cytoplasm and vesicles in axon terminals. Dopamine release from vesicles is triggered by action potential propagation-induced membrane depolarization. The axons of dopamine neurons extend the entire length of their designated pathway.

In one embodiment, the present invention provides a method of inducing aggregation of an alpha-synuclein (α-syn) protein in a cell including: contacting the cell with one of the vectors described herein; and exposing the cell to blue light illumination.

As used herein, “inducing aggregation of an α-syn protein” is meant to include the induction of the aggregation, the enhancement of the aggregation, and the acceleration of the process of aggregation of α-syn protein. In one aspect, inducing aggregation of an α-syn protein include contacting the cell with one of the vectors described herein to induce the expression of the fusion protein described herein. The isolated nucleic acid of the present invention may be introduced into a cell to be altered thus allowing expression of the fusion protein within the cell. A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion of a plasmid. Other methods of transfection include proprietary transfection reagents such as Lipofectamine™, Dojindo Hilymax™, Fugene™, jetPEI™, Effectene™ and DreamFect™.

The cell can be exposed to “blue light illumination”. As used herein, blue light illumination refers to any light having a wavelength of between approximately 380 nm and 500 nm. In some aspects, the blue light illumination includes illumination at 470 nm or at 488 nm.

The blue light illumination can be an acute pulsed blue light stimulation at a certain light intensity, frequency, and duration. The light intensity can be between 20 μW/mm² and 35 μW/mm². For example, the light intensity can be about 20, 21, 22, 23, 24, 25, 26, 27, 28, 29, 30, 31, 32, 34, or 35 μW/mm². In some aspects, the light intensity is about 26 μW/mm² or about 34 μW/mm². The light frequency can be between 0.1 Hz and 1 Hz. For example, the light frequency can be about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 Hz. In some aspects, the frequency is 0.17 Hz, 0.25 HZ, 0.5 Hz or 1 Hz. The pulsed blue light stimulation can be between a 0.1 and a 2 second pulse. For example, pulsed blue light stimulation can be a 0.1, 0.2, 0.3, 0.4, 0.5, 0.6. 0.7, 0.8, 0.9, 1, 1.5, or 2 s pulse. In many aspects, pulsed blue light stimulation includes 0.5 s pulse or 1 s pulse. The duration of the illumination can be between 30 min and 10 days or more. For example, the duration can be 30 min, 45 min, 1 h, 2 h, 5 h, 10 h, 12 h, 16 h, 20 h, 24 h, 48 h, 96 h, 4 days, 5 days, 6 days, 7 days, 8 days, 9 days, 10 days, or more, such as 2, 3, 4, 5, or more weeks. In some aspects, the duration is between about 1 hour and 7 days.

The fusion protein of the invention includes α-syn protein, a protein tag, and a light-responsive domain. Upon illumination of a cell including a nucleic acid sequence encoding such fusion protein (upon contacting of the cell with a vector including such nucleic acid sequence), light-induced dimerization of two light-responsive domains happens, which leads to the dimerization of two α-syn proteins. In turn, light-induced dimerization of two light-responsive domains of two dimers of α-syn can happens, and lead to the dimerization of two α-syn dimers. This process can repeat multiple time during the illumination of the cell, and progressively lead to the formation of α-syn protein aggregates (i.e., complexes including two or more α-syn fusion protein, interacting with one another through a light-responsive domain). Therefore, in one aspect, exposing the cell to blue light illumination generates α-syn aggregates. The intensity, frequency, and frequency of the illumination, as well as the duration of the illumination affect the ability of the -induced dimerization process to happen, as well as its speed. The longer a cell is exposed to blue light, the more α-syn protein aggregates will be generated. The shorter a cell is exposed to blue light, the less α-syn protein aggregates will be generated. In other aspects, exposing the cell to blue light illumination generates α-syn aggregates in a time and dose-dependent manner.

The α-syn aggregates can be located in any part of the cell, where α-syn is usually expressed in the cell. For example, the α-syn aggregates can be localized in the cytoplasm, in the nucleus, around the nucleus, in neurites, in the cell body (i.e., soma), in the dendrites, or in the axon. In some aspects, α-syn aggregates are located in a neurite region and/or in a cell body region of the cell. Native α-syn is a soluble protein, that becomes insoluble upon modification and aggregation. In pathologic conditions, α-syn is phosphorylated and generates pathological aggregates that are no longer soluble. Such insoluble aggregates are also referred to as Lewi bodies or Lewy neurites and correspond to abnormal collections of alpha-synuclein protein within brain neurons. Those clumps of protein form, neurons function less optimally and eventually die. Those α-syn aggregates are therefore pathological or pathogenic α-syn aggregates. There are various antibodies that are available for the detection of α-syn aggregates, that specifically recognized different forms of α-syn. For example, 5G4, Syn303 and Syn-O2 antibodies can be used to detect α-syn; pS129-α-Syn antibody can be used to detect pathological form of α-syn phosphorylated at S129; p62 antibody. ThioS, and ubiquitin antibodies can be used to detect p62, beta-sheet-containing amyloid, and ubiquitin, respectively, which are proteins known to interact and form aggregates with pathological α-Syn (i.e., those protein are part of the Lewi body aggregates).

The α-syn fusion protein of the invention, fused to a protein tag and to a light-responsive domain is soluble when non-aggregated (when the cells are not illuminated by blue light), and forms insoluble aggregates upon illumination by blue light. In many aspects, the α-syn aggregates are insoluble aggregates. In various aspects, the α-syn aggregates generate Lewi bodies in the cell. In one aspect, the α-syn aggregates are pathogenic α-syn aggregates. In various aspects, the α-syn aggregates include 5G4⁺, Syn-O2⁺, pS129⁺, Syn303⁺, p62⁺, ThioS⁺ and/or ubiquitin⁺ α-syn aggregates. In some aspects, the α-syn aggregates decrease cell survival.

In another embodiment, the invention provides a method of identifying an α-syn aggregation inhibitor including: (i) contacting a cell with one of the vectors described herein, (ii) contacting the cell with a test compound, (iii) exposing the cell from (ii) to blue light illumination, and measuring an aggregate induction score (AIS) of the test compound in the cell exposed to blue light illumination (blue AIS); and (vi) exposing a cell from (ii) to the dark and measuring an AIS of the test compound in the cell exposed to the dark (dark AIS), wherein an α-syn aggregation inhibitor has a greater blue AIS as compared to a dark AIS.

As used herein, an α-syn aggregation inhibitor refers to any compound (organic or inorganic) that can reduce, inhibit, slow down, block or interfere with the pathological aggregation of α-syn proteins, it can include compounds with no known function, that are identified through the method described herein as an α-syn aggregation inhibitor, or to compounds with a previously known functionality, for which the methods described herein identify a new function as an α-syn aggregation inhibitor.

As used herein, an aggregate induction score (AIS) is a score that reflect the number of aggregates present per cell. Cells expressing the α-syn fusion protein of the invention are incubated with a test compound or with a negative control (1% DMSO) and aggregation is induced by illuminating the cells with blue light. After fixation of the cells aggregated-α-syn were detected by immunofluorescence and multiples images are captured. To analyze images, the automated aggregation quantification algorithm and cell counting algorithm in the form of macro is used and the Aggregates Induction Score (AIS)s calculated using the following equation:

$$A I S ( Aggregates Induction Score ) = [ \frac{{N}^{agg}}{{N}^{total}} ] ,$$

where N^{agg} is the number of aggregates, N^{total} is the number of total cells. The AIS is normalized by the AIS in positive control which is set as 1.0. A hit selection strategy based on calculated AIS defines a compound as a hit if AIS<0.5.

In one aspect, an AIS is the ratio of a number of α-syn aggregates over a number of cells. In another aspect, an α-syn aggregation inhibitor inhibits or delays α-syn aggregation. In some aspects, an α-syn aggregation inhibitor has a blue AIS greater than 0.19.

In another aspect, Z′ values of the test compound are further measured. In some aspects, measuring Z′ values include calculating the degree of separation between the blue AIS and the dark AIS.

A cell including a nucleic acid sequence encoding an opto-α-syn fusion protein, contacted with a test compound is exposed independently to blue light illumination to measure an aggregate induction score (AIS) of the test compound in the cell exposed to blue light illumination (blue AIS); and to the dark to measuring an AIS of the test compound in the cell exposed to the dark (dark AIS). The cell, contacted with a control compound, such as DMSO, is also exposed to blue light illumination to measure a control aggregate induction score (AIS) of the cell exposed to blue light illumination (positive control AIS).

The positive control AIS reflect the optimal number of aggregates that can be generated in the cell when the cell is exposed to conditions that are favorable to the generation of α-syn aggregates (i.e., blue light). The blue AIS reflects the number of aggregates generated in the cell in the presence of the test compound, when the cell is exposed to conditions that are favorable to the generation of α-syn aggregates. The dark AIS reflects the number of aggregates generated in the cell in the presence of the test compound, when the cell is exposed to conditions that are not favorable to the generation of α-syn aggregates (internal negative control).

A blue AIS of a compound that is equivalent or greater than a positive control AIS indicates that, in the presence of the compound, the cell can generate equivalent amount or more α-syn aggregates, which indicates that the compound is not an α-syn aggregation inhibitor.

A blue AIS of a compound that is less than a positive control AIS, but more than a dark AIS, indicates that, in the presence of the compound, the cell can generate less α-syn aggregates, which indicates that the compound is a α-syn aggregation inhibitor.

A blue AIS of a compound that is less than a positive control AIS, and equivalent or less than a dark AIS, indicates that, in the presence of the compound, the cell cannot generate α-syn aggregates, which indicates that the compound is a potent α-syn aggregation inhibitor.

An α-syn aggregation inhibitor has a greater blue as compared to a dark AIS.

Insoluble α-syn aggregates are abnormal collections of alpha-synuclein protein within brain neurons responsible for the loss of neurons function less, and ultimately for neuron death. A α-syn aggregation inhibitor is a compound that inhibit, reduce, or decelerate the formation of α-syn aggregates, which are responsible for neuron death; therefore α-syn aggregation inhibitor can protect neuron from cell death. In other aspects, an α-syn aggregation inhibitor increases cell survival.

In an additional embodiment, the invention provides a method of inhibiting the formation of Lewi bodies in a cell including contacting the cell with an α-syn aggregation inhibitor identified by one of the methods described herein.

As used herein, the term “Lewy bodies” refers to the inclusion bodies—abnormal aggregations of protein—that develop inside nerve cells affected by Parkinson's disease (PD), the Lewy body dementias (Parkinson's disease dementia and dementia with Lewy bodies (DLB)), and some other disorders. They are also seen in cases of multiple system atrophy, particularly the parkinsonian variant (MSA-P). Lewy bodies appear as spherical masses in the cytoplasm that displace other cell components. For instance, some Lewy bodies tend to displace the nucleus to one side of the cell. There are two main kinds of Lewy bodies: classical and cortical. Lewy bodies may be found in the midbrain (within the substantia nigra) or within the cortex. A classical Lewy body is an eosinophilic cytoplasmic inclusion consisting of a dense core surrounded by a halo of 10 nm wide radiating fibrils, the primary structural component of which is alpha-synuclein.

A Lewy body is composed of the protein alpha-synuclein associated with other proteins, such as ubiquitin, neurofilament protein, and alpha B crystallin. Tau proteins may also be present, and Lewy bodies may occasionally be surrounded by neurofibrillary tangles. Lewy bodies and neurofibrillary tangles can occasionally exist in the same neuron, particularly in the amygdala.

Alpha-synuclein modulates DNA repair processes, including repair of DNA double-strand breaks (DSBs) by the process of non-homologous end joining. The repair function of alpha-synuclein appears to be greatly reduced in Lewy body bearing neurons, and this reduction may trigger cell death. Mutations are the reason behind their damaged repair function. Lewy bodies are believed to represent an aggresome response in the cell. When misfolded proteins aggregate, or clump together, many diseases are more likely to develop, including those that are associated with Lewy bodies. Aggregation is believed to occur when there is a high number of misfolded proteins in the ubiquitin-proteasome pathway, which are then brought to a resulting aggresome so they can be organized into one place. Since Lewy bodies are made of ubiquitinated proteins that would be handled in the ubiquitin-proteasome pathway, they may be made from this or a similar process if the pathway capacity is indeed exceeded by misfolded proteins that aggregate together. Accordingly, the aggresome, where the damaged proteins fully aggregate, is akin to the Lewy body.

As used herein, a α-syn aggregation inhibitor can be any organic or inorganic compound, including small molecules. For example, the small molecule can be a compound with an unidentified function, or a compound having a previously identified function. For example, the small molecule can be a compound active at GPCRs, kinases, ion channels, nuclear receptors, and transporters. In one aspect, the α-syn aggregation inhibitor is selected from Cyclothiazide, BVT 948, C1 976, NE 100 hydrochloride, BX 471, C 021 dihydrochloride, BAG 956, Arcyriaflavin A, Amlexanox, Kartogenin, Neuropathiazol, Napabucasin, Dexamethasone, XD 14, Mycophenolic acid, Cilostazol, Mycophenolate mofetil, Rizatriptan benzoate, or AZD 1480.

In yet another embodiment, the invention provides a method of treating a synucleinopathy in a subject including administering to the subject in need thereof an α-syn aggregation inhibitor identified by one of the methods described herein.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal, or human that is being sought by the researcher, veterinarian, medical doctor, or another clinician. Generally, the response is either amelioration of symptoms in a patient or a desired biological outcome (e.g., inhibition of α-syn aggregation, treatment of the synucleinopathy).

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters, and guinea pigs), cats, dogs, rabbits, farm animals including cows, horses, goats, sheep, pigs, chickens, etc., and primates (including monkeys, chimpanzees, orangutans, and gorillas) are included within the definition of subject.

As used herein, the term “synucleinopathy” refers to any disease or condition characterized by or having as a symptom the accumulation of α-syn aggregates in neuronal cells. α-syn aggregates form insoluble fibrils in pathological conditions characterized by Lewy bodies, such as Parkinson's disease, dementia with Lewy bodies and multiple system atrophy. Aggregation of α-syn lead to various cellular disorders including microtubule impairment, synaptic and mitochondrial dysfunctions, oxidative stress as well as dysregulation of calcium signaling, proteasomal and lysosomal pathway. Alpha-synuclein is the primary structural component of Lewy body fibrils. Occasionally, Lewy bodies contain tau protein; however, alpha-synuclein and tau constitute two distinctive subsets of filaments in the same inclusion bodies. Alpha-synuclein pathology is also found in both sporadic and familial cases with Alzheimer's disease.

In one aspect, the synucleinopathy is selected from the group consisting of Parkinson Disease (PD), dementia with Lewy body (DLB), multiple system atrophy (MSA), and neuroaxonal dystrophy.

The α-syn aggregation inhibitor identified by the methods described herein can be administered to a subject. The terms “administration of” and or “administering” should be understood to mean providing a pharmaceutical composition in a therapeutically effective amount to the subject in need of treatment. Administration routes can be enteral, topical, or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, nasal, ocular administrations, as well infusion, inhalation, and nebulization. The phrases “parenteral administration” and “administered parenterally” as used herein means modes of administration other than enteral and topical administration. One of skill in the art can easily identify the most appropriate route of administration based on the characteristics and properties of the α-syn aggregation inhibitor.

The α-syn aggregation inhibitor can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc. In some aspects, administration can be in combination with one or more additional therapeutic agents. The phrases “combination therapy”, “combined with” and the like refer to the use of more than one medication or treatment simultaneously to increase the response. The α-syn aggregation inhibitor of the present invention might for example be used in combination with other drugs or treatment in use to treat synucleinopathies. Such therapies can be administered prior to, simultaneously with, or following administration of the α-syn aggregation inhibitor of the present invention.

In one embodiment, the invention provides an optogenetic alpha-synuclein (α-syn) aggregation system including: (i) a LED illuminator; and (ii) a cell comprising a vector comprising a nucleic acid sequence encoding an α-syn fusion protein.

In one aspect, the fusion protein comprises in operable linkage an α-syn protein, a light-responsive domain, and a protein tag. In another aspect, the LED illuminator is a 12-channel, 24-channel, or 96-channel LED illuminator. In some aspects, the system further includes a LED excitation remote controller and a cell culture incubator.

In another embodiment, the invention provides a method of providing neuroprotective effects against a neurodegenerative disease in a subject including, administering to the subject BAG 956, thereby providing neuroprotective effects.

Neurodegenerative diseases are disorders that destroy motor neurons or their function. The methods described herein can be applied to any neurodegenerative disease. Exemplary neurodegenerative diseases include synucleinopathies, tauopathies, prion diseases, motor neuron diseases, dementia, transmissible spongiform encephalopathies, systemic atrophies primarily affecting the central nervous system, trinucleotide repeat disorders, proteinopathies, amyloidosis, neuronal ceroid lipofuscinoses, and others.

In some aspects, the methods described herein are used to provide neuroprotective effect against a synucleinopathy or tauopathy. Synucleinopathies are characterized by the abnormal accumulation of aggregates of α-synuclein in neurons, nerve fibers, or glial cells. Exemplary synucleinopathies include Parkinson's disease, dementia with Lewy bodies, multiple system atrophy (MSA), and certain neuroaxonal dystrophies. In some embodiments, the synucleinopathy is Parkinson's disease. Tauopathies are neurodegenerative diseases associated with the pathological aggregation of tau protein in neurofibrillary or gliofibrillary tangles in the human brain. Exemplary tauopathies include, but are not limited to, Alzheimer's disease, primary age-related tauopathy (PART), chronic traumatic encephalopathy (CTE), progressive supranuclear palsy (PSP), corticobasal degeneration (CBD), frontotemporal dementia and parkinsonism linked to chromosome 17 (FTDP-17), amyotrophic lateral sclerosis-parkinsonism-dementia (ALS-PDC, Lytico-bodig disease), ganglioglioma, gangliocytoma, meningioangiomatosis, postencephalitic parkinsonism, subacute sclerosing panencephalitis (SSPE), lead encephalopathy, tuberous sclerosis, pantothenate kinase-associated neurodegeneration, and lipofuscinosis.

In one aspect, providing neuroprotective effects includes inducing the clearance of alpha-synuclein (α-syn) aggregates, inducing the clearance of tau aggregates, and/or inducing autophagic flux and autophagic degradation of α-syn and/or tau aggregates.

In some aspects, inducing the clearance of alpha-synuclein (α-syn) aggregates or inducing the clearance of tau aggregates includes decreasing levels of insoluble pS129−, α-syn, pTau 202/205, pTau 231, pTau 217 and or AT8+ pTau in the subject.

In other aspects, inducing autophagic flux and autophagic degradation of α-syn and/or tau aggregates includes inhibiting PI3K/PDK1/AKT/mTor pathway in dopaminergic neurons, inducing LC3II expression, inhibiting p62 expression, and/or increasing LC3+ and/or LC3_5G4+ vesicles in dopaminergic neurons.

In another aspect, providing neuroprotective effects includes improving grip strength, locomotion and/or hippocampal/amygdala dependent learning and memory in the subject. In one aspect, providing neuroprotective effects includes inhibiting loss of TH+ neurons in the substantia negra.

In another aspect, administering comprises oral administration of BAG 956.

In some aspects, oral administration of BAG 956 includes oral administration of about 1-20 mg/kg.

For example, the oral administration of BAG 956 includes the oral administration of about 1 mg/kg, about 2 mg/kg, about 3 mg-kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg or about 15 mg/kg, of BAG 956.

In various aspects, oral administration of BAG 956 includes about 2 mg/kg or about 10 mg/kg.

### ADDITIONAL EMBODIMENTS

Embodiment 1: An isolated nucleic acid sequence comprising: a first nucleic acid sequence encoding an alpha-synuclein (α-syn) protein; a second nucleic acid sequence encoding a Cry2 PHR or a Cry2clust light-responsive domain; and a third nucleic acid sequence encoding a hemagglutinin (HA) tag or a mCherry tag, in operable linkage.

Embodiment 2: The isolated nucleic acid sequence of embodiment 1, wherein the light-responsive domain is fused at the C-terminus of the α-syn protein.

Embodiment 3: A plasmid comprising the nucleic acid sequence of any of embodiments 1-2.

Embodiment 4: A viral vector comprising the nucleic acid sequence of any of embodiments 1-2.

Embodiment 5: An isolated mammalian cell comprising the plasmid of embodiment 3 or the viral vector of embodiment 4.

Embodiment 6: The isolated cell of embodiment 5, wherein the cell is a terminally differentiated neuron, an induced pluripotent stem cell-derived neural progenitor cell (iPSC NPC), or an iPSC-derived midbrain dopaminergic (iPSC-derived mDA) neuron.

Embodiment 7: A method of inducing aggregation of an alpha-synuclein (α-syn) protein in a cell comprising: contacting the cell with a plasmid or a viral vector comprising an isolated nucleic acid sequence comprising: a first nucleic acid sequence encoding an alpha-synuclein (α-syn) protein; a second nucleic acid sequence encoding a Cry2 PHR or a Cry2clust light-responsive domain; and a third nucleic acid sequence encoding a hemagglutinin (HA) tag or a mCherry tag, in operable linkage; and exposing the cell to blue light illumination, thereby inducing aggregation of α-syn protein in the cell.

Embodiment 8: The method of embodiment 7, wherein the cell is an iPSC-derived midbrain dopaminergic (iPSC-derived mDA) neuron.

Embodiment 9: The method of embodiment 7 or 8, wherein the blue light illumination comprises illumination at 470 nm or at 488 nm.

Embodiment 10: The method of any one of embodiments 7-9, wherein exposing the cell to blue light illumination comprises exposing the cell to an acute pulsed blue light stimulation at a certain light intensity, frequency, and duration.

Embodiment 11: The method of embodiment 10, wherein the light intensity is about 26 μW/mm2 to 34 μW/mm2.

Embodiment 12: The method of embodiment 10 or 11, wherein the frequency is 0.17 Hz, 0.25 HZ, 0.5 Hz or 1 Hz.

Embodiment 13: The method of any one of embodiments 10-12, wherein pulsed blue light stimulation comprises 0.5 s pulse or 1 s pulse.

Embodiment 14: The method of any one of embodiments 10-13, wherein the duration is between about 1 hour and 7 days.

Embodiment 15: The method of any one of embodiments 10-14, wherein exposing the cell to blue light illumination generates α-syn aggregates.

Embodiment 16: The method of any one of embodiments 7-15, wherein exposing the cell to blue light illumination generates α-syn aggregates in a time and dose-dependent manner.

Embodiment 17: The method of any one of embodiments 7-16, wherein α-syn aggregates are located in a neurite region and/or in a cell body region of the cell.

Embodiment 18: The method of any one of embodiments 7-17, wherein the α-syn aggregates are insoluble aggregates.

Embodiment 19: The method of any one of embodiments 7-18, wherein the α-syn aggregates generate Lewi bodies in the cell.

Embodiment 20: The method of any one of embodiments 7-19, wherein the α-syn aggregates are pathogenic α-syn aggregates.

Embodiment 21: The method of any one of embodiments 7-20, wherein the α-syn aggregates comprise 5G4+, Syn-O2+, pS129+, Syn303+, p62+, ThioS+ and/or ubiquitin+ α-syn aggregates.

Embodiment 22: The method of any one of embodiments 7-21, wherein the α-syn aggregates decrease cell survival.

Embodiment 23: A method of identifying an α-syn aggregation inhibitor comprising: (i) contacting a cell with a plasmid or a viral vector comprising an isolated nucleic acid sequence comprising: a first nucleic acid sequence encoding an alpha-synuclein (α-syn) protein; a second nucleic acid sequence encoding a Cry2 PHR or a Cry2clust light-responsive domain; and a third nucleic acid sequence encoding a hemagglutinin (HA) tag or a mCherry tag, in operable linkage, (ii) contacting the cell with a test compound, (iii) exposing the cell from (ii) to blue light illumination, and measuring an aggregate induction score (AIS) of the test compound in the cell exposed to blue light illumination (blue AIS); and (vi) exposing a cell from (ii) to the dark and measuring an AIS of the test compound in the cell exposed to the dark (dark AIS), wherein an α-syn aggregation inhibitor has a greater blue AIS as compared to a dark AIS, thereby identifying α-syn aggregation inhibitor.

Embodiment 24: The method of embodiment 23, wherein the cell is an iPSC-derived midbrain dopaminergic (iPSC-derived mDA) neuron.

Embodiment 25: The method of embodiment 23 or 24, further comprising measuring Z′ values of the test compound.

Embodiment 26: The method of any one of embodiments 23-25, wherein measuring Z′ values comprises: calculating the degree of separation between the blue AIS and the dark AIS.

Embodiment 27: The method of any one of embodiments 23-26, wherein an AIS is the ratio of a number of α-syn aggregates over a number of cells.

Embodiment 28: The method of any one of embodiments 23-27, wherein an α-syn aggregation inhibitor inhibits or delays α-syn aggregation.

Embodiment 29: The method of any one of embodiments 23-28, wherein an α-syn aggregation inhibitor has a blue AIS greater than 0.19.

Embodiment 30: The method of any one of embodiments 23-29, wherein an α-syn aggregation inhibitor increases cell survival.

Embodiment 31: A method of inhibiting the formation of Lewi bodies in a cell comprising contacting the cell with an α-syn aggregation inhibitor identified by the method of any one of embodiments 23-30.

Embodiment 32: The method of embodiment 31, wherein the cell is a midbrain dopaminergic (mDA) neuron.

Embodiment 33: The method of embodiment 31 or 32, wherein the α-syn aggregation inhibitor is selected from Cyclothiazide, BVT 948, CI 976, NE 100 hydrochloride, BX 471, C 021 dihydrochloride, BAG 956, Arcyriaflavin A, Amlexanox, Kartogenin, Neuropathiazol, Napabucasin, Dexamethasone, XD 14, Mycophenolic acid, Cilostazol, Mycophenolate mofetil, Rizatriptan benzoate, or AZD 1480.

Embodiment 34: A method of treating a synucleinopathy in a subject comprising: administering to the subject in need thereof an α-syn aggregation inhibitor identified by the method of any one of embodiments 23-30.

Embodiment 35: The method of embodiment 34, wherein the α-syn aggregation inhibitor is selected from Cyclothiazide, BVT 948, CI 976, NE 100 hydrochloride, BX 471, C 021 dihydrochloride, BAG 956, Arcyriallavin A, Amlexanox, Kartogenin, Neuropathiazol, Napabucasin, Dexamethasone, XD 14, Mycophenolic acid, Cilostazol, Mycophenolate mofetil, Rizatriptan benzoate, or AZD 1480.

Embodiment 36: The method of embodiment 34 or 35, wherein the synucleinopathy is selected from the group consisting of Parkinson Disease (PD), dementia with Lewy body (DLB), multiple system atrophy (MSA), and neuroaxonal dystrophy.

Embodiment 37: An optogenetic alpha-synuclein (α-syn) aggregation system comprising: (i) a LED illuminator, and (ii) a cell comprising a vector comprising a nucleic acid sequence encoding an α-syn fusion protein.

Embodiment 38: The system of embodiment 38, wherein the fusion protein comprises in operable linkage an α-syn protein, a light-responsive domain, and a protein tag.

Embodiment 39: The system of embodiment 37 or 38, wherein the LED illuminator is a 12-channel, 24-channel, or 96-channel LED illuminator.

Embodiment 40: The system of any one of embodiment 37-39, further comprising a LED excitation remote controller and a cell culture incubator.

Embodiment 41: A method of providing neuroprotective effects against a neurodegenerative disease in a subject comprising administering to the subject BAG 956, thereby providing neuroprotective effects.

Embodiment 42: A method of inducing the clearance of alpha-synuclein (α-syn) aggregates in a subject having a neurodegenerative disease comprising administering to the subject BAG 956, thereby inducing the clearance of α-syn.

Embodiment 43: A method of inducing the clearance of tau aggregates in a subject having a neurodegenerative disease comprising administering to the subject BAG 956, thereby inducing the clearance of tau aggregates.

Embodiment 44: The method of embodiment 42 or 43, wherein inducing the clearance of alpha-synuclein (α-syn) aggregates or inducing the clearance of tau aggregates comprises decreasing levels of insoluble pS129−, α-syn, pTau 202/205, pTau 231, pTau 217 and/or AT8+ pTau in the subject.

Embodiment 45: A method of inducing autophagic flux and autophagic degradation of α-syn and/or tau aggregates in a subject having a neurodegenerative disease comprising administering to the subject BAG 956, thereby inducing autophagic flux and autophagic degradation of α-syn and/or tau aggregates.

Embodiment 46: The method of embodiment 45, wherein inducing autophagic flux and autophagic degradation of α-syn and/or tau aggregates comprises inhibiting PI3K-PDK1/AKT/mTor pathway in dopaminergic neurons, inducing LC3II expression, inhibiting p62 expression, and/or increasing LC3+ and/or LC3_5G4+ vesicles in dopaminergic neurons.

Embodiment 47: A method of improving grip strength, locomotion and/or hippocampal/amygdala dependent learning and memory in a subject having a neurodegenerative disease comprising administering to the subject BAG 956, thereby improving grip strength, locomotion and/or hippocampal/amygdala dependent learning and memory.

Embodiment 48: A method of inhibiting loss of TH+ neurons in the substantia negra in a subject having a neurodegenerative disease comprising administering to the subject BAG 956, thereby inhibiting loss of TH+ neurons in the substantia negra.

Embodiment 49: The method of any one of embodiments 41-48, wherein the neurodegenerative disease is a synucleinopathy or a tauopathy.

Embodiment 50: The method of any one of embodiments 41-49, wherein the neurodegenerative disease is a synucleinopathy selected from the group consisting of Parkinson Disease (PD), dementia with Lewy body (DLB), multiple system atrophy (MSA), and neuroaxonal dystrophy.

Embodiment 51: The method of any one of embodiments 41-50, wherein the synucleinopathy is Parkinson's disease.

Embodiment 52: The method of any one of embodiments 41-49, wherein the neurodegenerative disease is a tauopathy selected from the group consisting of Alzheimers disease (AD), primary age-related tauopathy (PART)/Neurofibrillary tangle-predominant senile dementia, chronic traumatic encephalopathy (CTE), progressive supranuclear palsy (PSP), corticobasal degeneration (CBD), frontotemporal dementia and parkinsonism linked to chromosome 17 (FTDP-17), lytico-bodig disease (Parkinson-dementia complex of Guam), ganglioglioma and gangliocytoma, meningioangiomatosis, postencephalitic parkinsonism, subacute sclerosing panencephalitis (SSPE), lead encephalopathy, tuberous sclerosis, pantothenate kinase-associated neurodegeneration, and lipofuscinosis.

Embodiment 53: The method of any one of embodiments 41-51, wherein administering comprises oral administration of BAG 956.

Embodiment 54: The method of any one of embodiments 41-52, wherein oral administration of BAG 956 comprises oral administration of about 1-20 mg/kg.

Embodiment 55: The method of any one of embodiments 41-53, wherein oral administration of BAG 956 comprises about 2 mg/kg or about 10 mg/kg.

Presented below are examples discussing optogenetic α-syn fusion protein and uses thereof contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Material and Methods

### Plasmid Construction and Transfection

Cry2 PHR coding sequence from pmCitrine-opto-FGFR 1 (Kim et al., 2014) (gift from Won Do Heo) was subcloned into pHM6-HA-α-syn (Addgene plasmid #40824, a gift from David Rubinsztein) to generate either pHM6-HA-α-syn-Cry2 PHR (pHM6-opto-α-syn) or pHM6-HA-Cry2 PHR (pHM6-opto-mock). Cry2clust coding sequence was from mCherry-CRY2clust (Addgene plasmid #105624). α-syn-mCherry-Cry2clust or mCherry-Cry2clust were synthesized by GenScript (Piscataway, NJ, USA). The dsDNA donor vectors for homologous recombination at the AAVS1 locus are designed to have either SA-2A-Puro^{R}-CAG-HA-α-syn-PHR (for AAVS1::HA-opto-α-syn), SA-2A-Puro^{R}-CAG-HA-PHR (for AAVS1::HA-opto-mock), SA-2A-Puro^{R}-CAG-α-syn-mCherry-Cry2clust (for AAVS1::opto-α-syn) or SA-2A-Puro^{R}-CAG-mCherry-Cry2clust (for AAVS1::opto-mock) gene cassettes between both homology arms, using AAV-CAGGS-EGFP (Addgene plasmid #22212, a gift from RudolfJaenisch) as a backbone. Each homology arm has 804 bp (AAVS1 left arm) or 837 bp (AAVS1 right arm) sequences in the fist intron of PPP1R12C. A gRNA target sequence for AAVS1 was chosen to have the same sequence as that of gRNA_AAVS1-T1 (Mali et al., 2013) (Addgene plasmid #41817, a gift from George Church) and subcloned into PX458 (hCas9/gRNA, Addgene plasmid #48138, a gift from Feng Zhang). The oligonucleotides for the PX458-AAVS1 construct were as follows: forward 5′-CACCGTCCCCTCCACCCCACAGTG-3′(SEQ ID NO:2) and reverse 5′-AAACCACTGTGGGGTGGAGGGGAC-3′ (SEQ ID NO:3). All insert sequences were verified by Sanger DNA sequencing (JHU Synthesis & Sequencing Facility). Plasmid transfections were performed using LIPOFECTAMINE® LTX and PLUS™ Reagent (Invitrogen) according to the manufacturer's instructions.

### SH-SY5Y Cell Culture and Neuronal Differentiation

SH-SY5Y cells were grown in culture medium containing DMEM/F-12, 15% heat-inactivated FBS, 2 mM L-glutamine, and 1% penicillin/streptomycin (all from Life Technologies). For the neuronal differentiation, we followed a previously described protocol. Briefly, undifferentiated SH-SY5Y cells were plated on uncoated dishes in reduced-serum (2.5% or 1%) culture media supplemented with 10 jiM RA (Sigma-Aldrich) and media was changed on every other day until day 10. Then cells were split to Geltrex (Life Technologies)-coated dishes in Neurobasal medium (Life Technologies) supplemented with 10 jiM RA, 2 mM L-glutamine, 1% penicillin/streptomycin, B-27 (Life Technologies), 2 mM dbcAMP (Sigma-Aldrich) and 50 ng/mL BDNF (PeproTech). The cells were terminally differentiated into neurons at day 18.

### Generation of Knock-In SH-SY5Y Cell Lines by Using Homologous Recombination

After SH-SY5Y cells reached 90% confluence in 10 cm dishes, they were transfected with 5 μg hCas9/gRNA and 15 jig donor plasmids for AAVS1::opto-α-syn or AAVS1::opto-mock (see Plasmid construction and transfection) using Lipofectamine® LTX and Plus™ Reagent according to the manufacturer's instructions. Two days after the transfection, the cells were re-plated into 10 cm dishes, and then the cells that had undergone homologous recombination were selected with the 2 jig/mL puromycin containing culture media for a week. Surviving cells were cultured for another 8 weeks to form single colonies.

Generation of Knock-In hiPSC Lines by Using Homologous Recombination

The feeder-free SNCA triplication PD hiPSCs (ND27760-8) were dissociated into single cells using Accutase (innovative Cell Technologies), and 2×10⁶ cells were resuspended in nucleofection solution V (Lonza) with 10 jig hCas9/gRNA and 10 jig donor plasmids for AAVS1 . . . opto-α-syn (see Plasmid construction and transfection). Nucleofection was performed with Nucleofector™ II according to the manufacturer's instruction (using the B-16 program, Lonza). The nucleofected cell suspension was subsequently plated on puromycin-resistant MEFs (DR4, Global Stem) in hESC medium with 10 jiM Y-27632. Four days after nucleofection, the cells that had undergone homologous recombination were selected by adding 0.5 jig/ml of puromycin to hESC medium for four days.

hiPSC Culture and mDA Neuronal Differentiation

We cultured undifferentiated SNCA triplication PD hiPSCs (ND27760-8) (Devine et al., 2011) and opto-α-syn (AAVS1 . . . opto-α-syn) PD hiPSCs on mitotically inactivated mouse embryonic fibroblasts (MEFs, Global Stem or Applied Stem Cell), in hESC medium containing DMEM/F-12, 20% knockout serum replacement (KSR), 0.1 mM MEM-NEAA, 2 mM L-glutamine, 55 jiM 3-mercaptoethanol (all from Life Technologies) and 10 ng/mL FGF2 (R&D Systems) as used routinely for iPSC cultures. All cells were maintained at 37° C. and 5% CO₂ in a humidified incubator. For mDA neuron differentiation, we used previously described methods of mDA neuron induction and neural progenitor cell expansion. Briefly, dissociated hiPSCs were plated on Geltrex at a density of 50,000 cells/cm² in MEF-conditioned KSR medium containing DMEM/F-12, 20% KSR, 0.1 mM MEM-NEAA, 2 mM L-glutamine, and 55 jiM 3-mercaptoethanol with 10 ng/mL FGF2 and 10 jiM ROCK-inhibitor (Y-27632, Cayman Chemical). After confluency of the cells reached 80%-90%, differentiation was initiated by switching to KSR medium supplemented with 100 nM LDN193189 (STEMCELL Technologies) and 10 μM SB431542 (Cayman Chemical). Supplements of 100 ng/mL Shh (C2511, R&D), 2 μM Purmorphamine (PMP, Cayman Chemical) and 100 ng/mL FGF8 (PeproTech) were added on days from 1 to 7, and 3 mM CHIR99021 (CHIR, Tocris) was added at day 3 to day 11. Beginning on day 5, the KSR medium was gradually replaced with increasing amounts of N2 medium (Oh et al., 2016) (25% increments every other day). To expand neural progenitors, the cells were split on Geltrex and maintained in medium containing DMEM/F-12, N-2 supplement (Life Technologies), 2 mM L-glutamine, 1% penicillin/streptomycin, 100 nM LDN193189, 3 μM CHIR and 10 μM Y-27632 on day 11. After that, the cells were re-plated on dishes pre-coated with Geltrex in NB/B-27 medium supplemented with 3 μM CHIR, 20 ng/mL BDNF, 0.2 mM ascorbic acid, 20 ng/mL GDNF, 1 ng/mL TGFβ3, 0.5 mM dbcAMP and 10 μM DAPT for at least 10 days to complete differentiation.

### Blue Light Illumination

A customized blue light illumination plate (TouchBright W-Series) was designed and manufactured by Live Cell Instrument (Seoul, Korea). This plate contained 17 LEDs (70 mW per LED) per well on a 12-well plate. The light intensity, frequency, and duration were controlled by customized software (Live Cell Instrument). The actual light intensity at 470 nm to the cell plate was measured by Laser Check (Coherent). The light intensity at the maximal output in 12-well, 24-well, and 96-well plates was 34 μW/mm², 34 μW/mm², and 26 μW/mm², respectively.

### Immunocytochemistry

The cells were fixed in 4% paraformaldehyde (PFA) and stained with the primary antibodies (listed below) after permeabilization with 0.1% Triton X-100/0.5% BSA in PBS solution. To examine the detergent-insoluble α-syn aggregates, the cells were fixed with 4% PFA containing 1% Triton X-100 for 15 min to remove soluble proteins. The appropriate Alexa Fluor 488-, 568-, or 647-labeled secondary antibody (Life Technologies) and DAPI (Roche Applied Science) nuclear counter-staining were used for visualization. The stained samples were analyzed using fluorescence microscopy (Eclipse TE2000-E, Nikon). The numbers of aggregate⁺, pS129-α-syn⁺, DAPI⁺, or transfected cells were counted under fluorescence microscopy. The primary antibodies used in this study are as follows with the target (clone), manufacturer, catalog number, isotype, and dilution specified, respectively: α-Syn (42/α-Synuclein), BD Transduction Laboratories, 610786, mouse IgG₁, and 1/1000; α-Syn (5G4), Millipore, MABN389, mouse IgG₁, and 1/1000; α-Syn (Syn303), BioLegend, 824301, mouse IgG₁, and 1/500; α-Syn (Syn-O2), BioLegend, 847602, mouse IgG₁, and 1/500; pS129-α-Syn (P-syn/81A), BioLegend, 825701, mouse IgG₂ₐ, and 1/1000; pS129-α-Syn (EP1536Y), Abcam, ab51253, rabbit IgG, and 1/1000; GFP, Abeam, ab13970, chicken IgY, and 1/1000; HA (16B12), BioLegend, 901501, mouse IgG₁, and 1/1000; HA, Abcam, ab9110, rabbit IgG, and 1/1000; HA (Poly9023), BioLegend, 902301, rabbit IgG, and 1/1000; TH, Pel-Freez Biologicals, P40101-150, rabbit IgG, and 1/1000; TUJ1, BioLegend, MMS-435P, mouse IgG₂ₐ, and 1/1000; mCherry, BioLegend, 677701, mouse IgG₂ₐ and 1/1000; mCherry, MilliporeSigma, AB356482, rabbit, and 1/1000; p62, MilliporeSigma, P0067, and 11500; and Ubiquitin, DAKO, Z0458, rabbit IgG, and 1/500. For Thioflavin S staining, the fixed cells were incubated in 0.1% (w/v) Thioflavin S (Sigma) solution for 8 min and were then washed with 50% ethanol for 5 min.

### Live-Cell Imaging

All live-cell imaging experiments were performed on Zeiss AxioObserver inverted microscope with LSM800 confocal module equipped with a stagetop incubator utilizing an oil immersion objective (Zeiss Plan-Neofluar 40×1.30 N.A., DIC). Following differentiation into mDA neurons, cells were equilibrated on the preheated (37° C. and 5% CO₂) stagetop incubator for 10 min prior to imaging. Acute blue light stimulation was achieved by utilizing the 488 nm laser and the stimulation module within ZEN imaging software. Stimulation with blue light varied from 1-5 s and laser power was 1% (1.5 μW). Following 5 baseline images, laser stimulation was performed, and cells were imaged for up to each indicated time of post-activation. Data presented are representative of at least two independent experiments utilizing three or more biological replicates per experiment.

### Quantitative Image Analysis

Analysis of immunostained images was performed by ImageJ software (NIH). Images were converted to grayscale, and a threshold was adjusted to exclude the background based on the size of particles. Setting a threshold was also used for accomplishing desired intensity values for each experiment. Once a threshold value was determined, all the images in each experiment were applied with the fixed threshold value, and then the number and the total area of immuno-positive aggregates per field were measured using the measurement function.

### Western Blot Analysis

The cells were lysed in RIPA buffer (Cell Signaling Technology) supplemented with 1% SDS (Amersco), 10% glycerol (Sigma-Aldrich), 1×Protease/Phosphatase Inhibitor Cocktail (Cell Signaling Technology), and 1 mM PMSF (Cell Signaling Technology). After sonicating to reduce the viscosity, cell lysates were mixed with Benzonase (Sigma-Aldrich) and incubated for 15 min at 37° C. The samples were clarified by centrifugation at 15,000 g for 30 min at 14° C., boiled at 98° C. for 2 min in Laemmli sample buffer (Sigma-Aldrich) supplemented with 20 mM DTT (Sigma-Aldrich), resolved by SDS-PAGE, and transferred to nitrocellulose membranes Bio-Rad). The western blot analyses were performed with the following antibodies with the target (clone), company, catalog number, isotype, and dilution specified, respectively: β-Actin, Cell signaling Technology, 8H10D10, mouse IgG₂ₐ, and 1/5000; HA (16B12), BioLegend, 901501, mouse IgG₁, and 1/1000.

### Primary Screen, Validation, and Hit Selection

The opto-α-syn expressing SH-SY5Y cells were seeded in 96-well black flat bottom imaging microplates (Falcon) at 30,000 cells per well in 100 μL of complete media using E1-ClipTip electronic multichannel pipette (Thermo Fisher Scientific) and incubated in 37° C. and 5% CO₂ humidified incubator. After 18 h of incubation, 10 μL of 10 μM compounds (column 2 to 10) or 1% DMSO (column 1 and 12) were added (final concentration of DMSO is 0.1%). In order to induce aggregations, the plates were illuminated with blue light (26 μW/mm²) on customized blue light illumination 96-well plates for 2 h. Afterward, cells were fixed in 4% paraformaldehyde (PFA) for 15 min and stained with the aggregated-α-syn antibody (5G4) after permeabilization with 0.1% Triton X-100/0.5% BSA/PBS solution. The Alexa Fluor 488 secondary antibody and DAPI nuclear counter-staining were used. After staining, every four images per well of the stained samples were captured automatically using BD Pathway™ 855 Bioimager for High-Content cell analysis. To analyze images, the automated aggregation quantification algorithm and cell counting algorithm in the form of macro are developed using ImageJ software. Briefly, the algorithm includes the inversion, subtracting background, threshold selection, analyzing particles with ranged size and circularity. The Aggregates Induction Score (AIS) is calculated using, the following equation:

$$A I S ( Aggregates Induction Score ) = [ \frac{{N}^{agg}}{{N}^{total}} ] ,$$

where N^{a}_{gg} is the number of aggregates, ^{Ntotal} is the number of total cells. The AIS in each well is normalized by the AIS in positive control which is set as 1.0. We applied the developed algorithms and calculated AIS for all samples to nominate candidate hits out of the 1,280 compounds. The hit selection strategy was based on calculated AIS; hits were defined as AIS<0.5. The 31 compounds fulfilled those criteria, but 12 compounds with too low cell numbers were excluded as possible compounds due to exhibiting toxicity. Remaining 19 potential hits were further validated in 24-well plates; 5 images per well were taken randomly. Two independent experiments were performed, and total 10 images per well are analyzed to calculate AIS. Finally, 5 compounds were chosen as AIS<0.5 and P<0.0001.

### Chemogenomic Analysis

The similarity ensemble approach (SEA) library search tool was used to identify target proteins of each compound via input of isomeric SMILES. Predicted targets were filtered with criteria of interaction p-values<0.05, selecting human targets, and compared with human protein atlas (HPA) to filtrate targets which are expressed in the human brain (Human Protein Atlas available from www.proteinatlas.org). Then targets which were targeted by over two compounds were selected to focus on the shared pathway across all the compounds. The Gene Ontology (GO) enrichment analysis was performed using g:Profiler (version e99_eg46_p14_f929183) with g:SCS multiple testing correction method applying significance threshold of 0.05 with selected targets.

### RNA Sequencing

Total RNA from 8 samples of PD-iPSCs derived mDA neurons with four different conditions were analyzed by Macrogen (Cambridge, MA). These datasets included two biological replicates. RNA extracts from cells under dark condition with DMSO and blue light stimulated condition treated with DMSO or 1 μM BAG, CDC for 24 h were subjected to cDNA library construction (TruSeq RNA Sample Prep Kit v2). The samples were checked for quality using FastQC v0.11.7 and then subjected to Illumina sequencing using the HiSeq 40(0) system. We aligned the sequencing reads to the reference genome using HISAT2 2.1.0 and bowtie2 2.3.4.1. We used DESeq2 R library to identify differentially expressed genes (p-value<0.05 and fold change cutoff of >1.5) between samples. We used the Kyoto Encyclopedia of Genes and Genomes (KEGG) database to determine the pathways of the differentially expressed genes. Gene Ontology enrichment of the differentially expressed genes was analyzed with DAVD using Fisher's exact test with the threshold of significance set by p-value. The data discussed in this publication have been deposited in NCBI's Gene Expression Omnibus and are accessible through GEO Series accession number “GSE153325”.

### Chemical Library

The library used for the screen contains 1,280 chemicals obtained from Tocris Bioscience. Tocriscreen™ compounds library has the collections of unique and diverse bioactive compounds suitable for high-throughput screening (HTS), cell-based high-content screening (HCS) and chemical biology applications including high purity compounds active at GPCRs, kinases, ion channels, nuclear receptors, and transporters.

### Statistical Analysis

The Z′ factor was used to assess assay performance. The Z′ factor constitutes a dimensionless parameter that ranges from 1 (infinite separation) to <0. It is defined as: Z′=1−(3σ_{c+}+3σ_{c-})/|μ_{c+}−μ_{c-}|, where σ_{c±}, σ_{c-}, μ_{c+}, and μ_{c-} are the standard deviations (σ) and averages (μ) of the positive control (c+, blue light illuminated opto-α-syn SH-SY5Y cells treated with 0.1% DMSO) and the negative control (c−, opto-α-syn SH-SY5Y cells in dark treated with 0.1% DMSO). Z′ factor between 0.5 and 1 indicates an excellent assay with good separation between controls. Z′ factor between 0 and 0.5 indicates a marginal assay, and <0 signifies a poor assay with no separation between controls. All data are represented as the mean±SEM or SD. The statistical analysis was performed using Prism 6 (GraphPad). The differences among multiple means were assessed by ANOVA followed by Tukey's or Dunnett's post hoc test. Assessments with P<0.05 were considered significant. Spearman's correlation coefficients (r) were pair-wisely estimated to compare the linearity between the two groups out of the three groups for the two different measurements, adjusted p-values and Gene Ratios; and the statistical significances of the correlation coefficients were tested at α=0.05. The statistical analyses were performed with SAS 9.4 (SAS Institute Inc, NC, USA). According to Akoglu (2018), when absolute value of r ranges from 0.6 to 0.7, it is interpreted as “moderate” and when it is greater than or equal to 0.8, it is interpreted that “very strong” linearity exists between the two (Akoglu, 2018).

### Example 2

### Light-Induced Pathogenic α-syn Aggregation in the Neuronal Cells

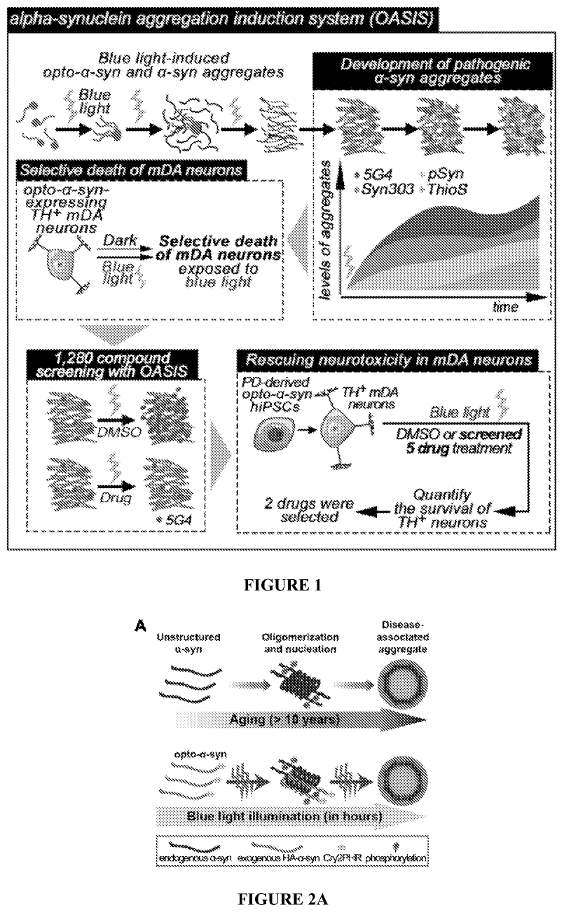

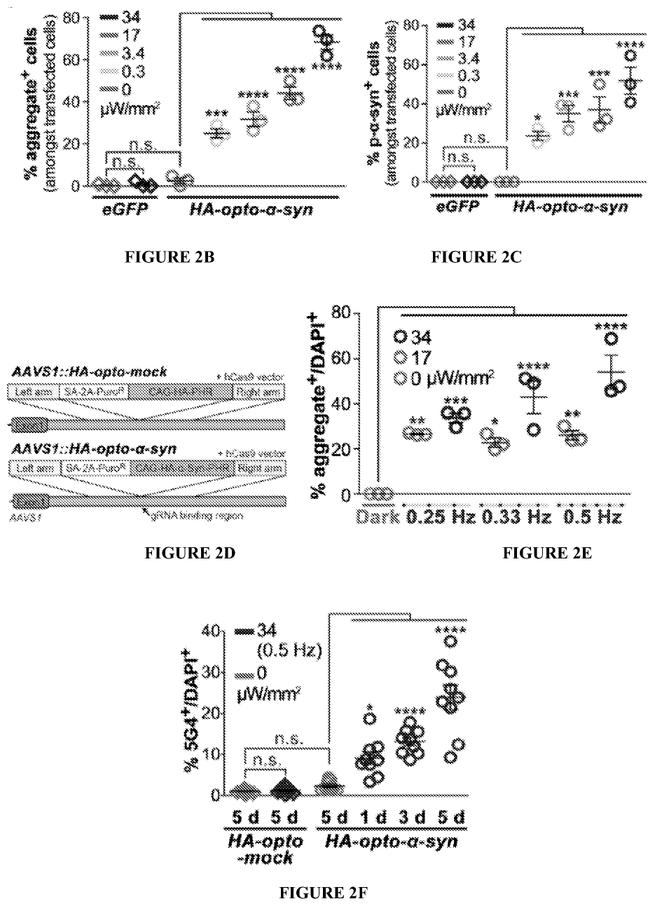

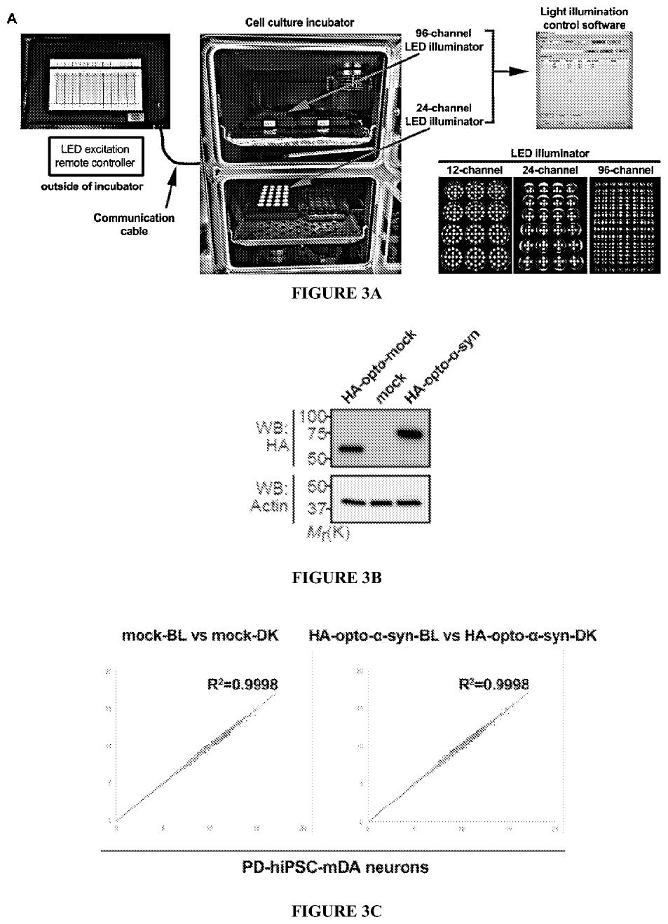

It was hypothesized that the use of an optogenetic modulation to increase the spatial proximity of α-syn monomer in neuronal cells can reproduce the formation of the disease-related α-syn aggregates, which is the pathogenic hallmark in PD. To develop an optogenetic α-syn aggregation system (as illustrated in FIG. 1), a light-responsive domain (Cry2 PHR of Arabidopsis thaliana) which promotes homo-interaction upon blue light illumination was introduced into HA-tagged α-syn (named HA-opto-α-syn, FIG. 2A). First, HA-opto-α-syn was transiently expressed in human neuronal SH-SY5Y cells and whether the blue light can induce its aggregation by using a customized blue light illumination plate was examined (FIG. 3A). The blue light illumination led to α-syn aggregation in an intensity-dependent manner, and the optically induced α-syn aggregates were also phosphorylated at S129, which is one of the important pathogenic markers of α-syn aggregates (FIGS. 2B and 2C). To explore the long-term effects of optical induction of α-syn aggregation, an HA-opto-α-syn knock-in (AAVS1::HA-opto-α-syn) SH-SY5Y cell line using CRISPR/Cas9 system (FIGS. 2D and 3B) was established. The optical induction of α-syn aggregation after 18 h of pulsed blue light (0.25 to 0.5 Hz, 0.5 s; FIG. 2E) was detected. The light-induced α-syn aggregates were also detected by immunostaining with different anti-HA antibodies, confirming that these findings were not caused by an artifact of antibody cross-reactivity. Interestingly, the α-syn aggregates were labeled with synucleinopathy-specific antibodies recognizing either aggregated (5G4, Syn-O2-) or misfolded (Syn303)-α-syn in the terminally differentiated neurons derived from HA-opto-α-syn SH-SY5Y cell line. Next, the disease-associated α-syn aggregates were quantified using 5G4 antibody at multiple time points and it was observed that the 5G4⁺ α-syn aggregates were gradually augmented with exposure to blue light over time (FIG. 2F). These disease-associated 5G4⁺ aggregates were also co-immunostained with two different antibodies specific for pS129. In addition, 5G4− labelled α-syn aggregates co-localized with pS129 or ubiquitin were insoluble in 1% Triton X-100. These results suggest that a controllable pathogenic OASIS on human neuronal cells was successfully developed.

As illustrated in FIG. 2A, light induced α-syn aggregation in cells. The opto-aggregation system schematically represented in FIG. 2A was used to accelerate and precisely control the formation of disease-associated α-syn aggregate. SH-SY5Y cells were transfected with HA-α-syn-Cry2 PHR (HA-opto-α-syn) or eGFP for 24 h in dark and then kept in dark or exposed to blue light continuously for 30 min. Transfected cells, with or without blue light illumination (0.34 to 34 μW/mm² at 470 nm), were co-immunostained with anti-HA or GFP and phospho-S129-α-syn (pS129, P-syn/81A) antibodies, imaged, and the percentage of aggregate⁺ (FIG. 2B) or phosphorylated-α-syn⁺ (p-α-syn⁺) cells (FIG. 2B), relative to the number of transtected cells was quantified using one-way ANOVA followed by Tukey's post hoc test (n=3). Homologous recombination enhanced by CRISPR/Cas9 system was used for AAVS1 locus targeting, as schematically pictured in FIG. 2D. The AAVS1::HA-opto-α-syn SH-SY5Y cells were kept in dark or exposed to pulsed blue light (17 or 34 μW/mm² at 470 nm, 0.25 to 0.5 Hz, 0.5 s) for 18 h. These cells were immunostained with anti-HA antibody and subjected to quantification of the percentage of aggregate⁺ cells, relative to the number of DAP⁺ cells (FIG. 2E), using one-way ANOVA followed by Tukey's post hoc test (n=3). Terminally differentiated AAVS1::HA-opto-α-syn SH-SY5Y cells were exposed to pulsed blue light (34 μW/mm² at 470 nm, 1 Hz, 0.5 s) for 2 h and then immunostained with 5G4, Syn-O2 or Syn303 antibody. Terminally differentiated AAVS1::H1A-opto-mock and AAVS1::HA-opto-α-syn SH-SY5Y cells were kept in dark or exposed to pulsed blue light (34 μW/mm² at 470 nm, 0.5 Hz, 0.5 s) for one to five days as indicated (FIG. 2F). These cells were immunostained with 5G4 antibody and subjected to quantification of the aggregated-α-syn (FIG. 2F). One-way ANOVA followed by Tukey's post hoc test (n=9, 3 images each from 3 independent experiments). Terminally differentiated AAVS1::11A-opto-α-syn SH-SY5Y cells were exposed to pulsed blue light (34 μW/mm² at 470 nm, 1 Hz, 0.5 s) for 3 h (left) or 20 h (right), and then immunostained with the indicated antibodies. Terminally differentiated AAVS1::HA-opto-α-syn SH-SY5Y cells were exposed to pulsed blue light as indicated for five days, and then fixed with 4% PFA containing 1% Triton X-100 for 15 min. These cells were co-immunostained with 5G4 and pS129-α-syn (P-syn/81A) or ubiquitin antibodies. Error bars represent mean±SEM. n.s., not significant. *P<0.05, **P<0.01, ***P<0.001, *****P<0.0001.

As illustrated in FIG. 3A, blue light illumination of the plates was done in a customized CO₂ cell culture incubator. Each channel of LEDs is remotely controlled by the LED excitation controller through a communication cable. Duration of blue light can be regulated by the light illumination control software. The maximum blue light intensity of 12-, 24-, and 96-channel is 34 μW/mm², 34 μW/mm², and 26 μW/mm², respectively. Mock-, AAVS1::HA-opto-mock, or AAVS1::HA-opto-α-syn SH-SY5Y cells were lysed with RIPA buffer and then subjected to immunoblot analysis with anti-HA antibody. Actin was used as a loading control (FIG. 3B). The AAVS1::HA-opto-α-syn SH-SY5Y cells were exposed to pulsed blue light (34 μW/mm² at 470 nm, 1 Hz, 0.5 s) for 2 h and then immunostained with the indicated anti-HA antibodies.

### Example 3

### Light-Induced Disease-Associated α-Syn Aggregation in PD hiPSC-Derived mDA Neurons

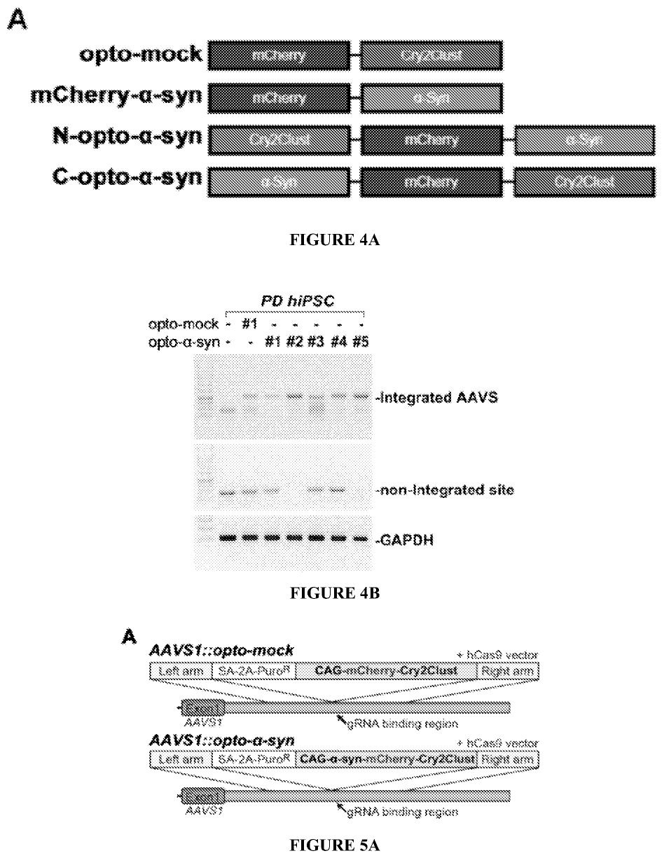

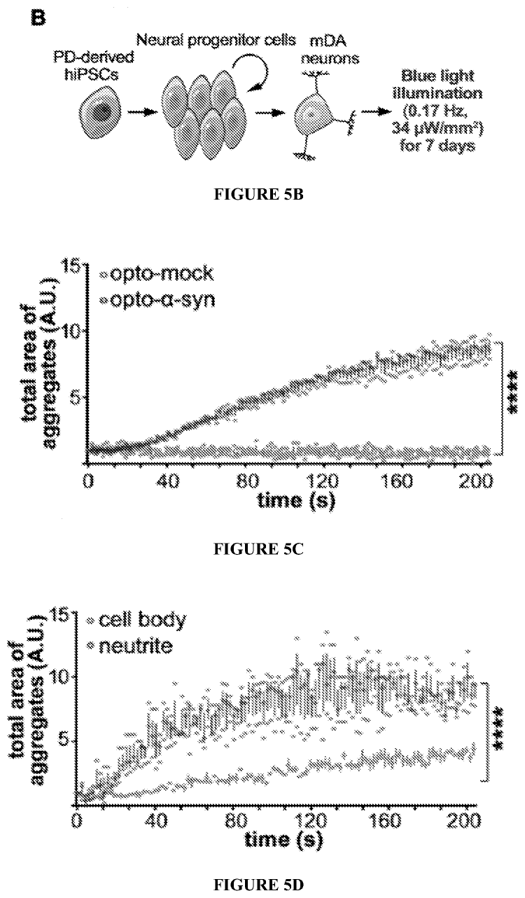

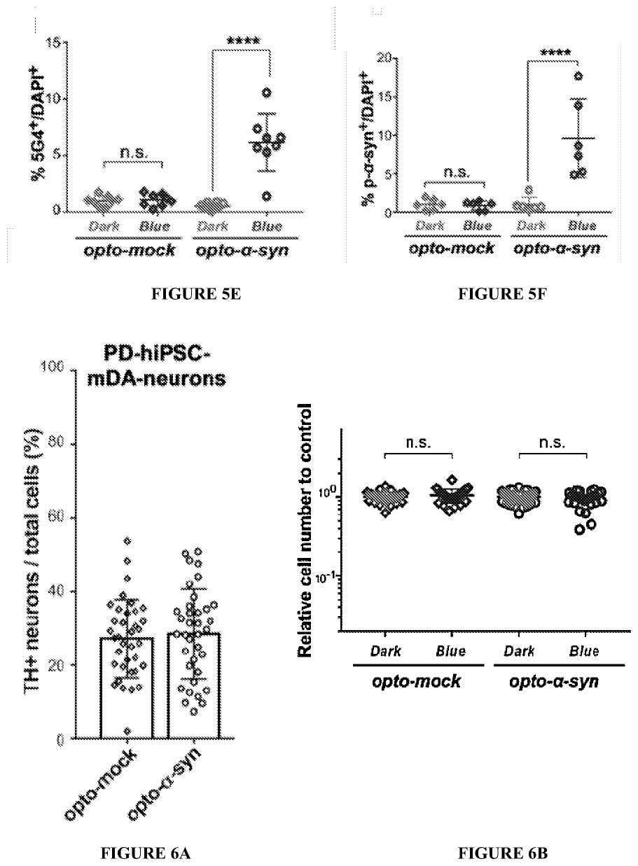

The pathological α-syn species in PD hiPSC-derived mDA neurons without extrinsic stress has been shown once; however, the spatiotemporal induction of α-syn aggregation cannot be regulated with the previously used methods. Adapting Cry2 PHR-based OASIS to PD hiPSC-derived mDA neurons was attempted, but neither distinct aggregate or significant differences of whole-transcriptome between the samples with or without blue light illumination could be observed (FIG. 3C). To improve light-mediated homo-oligomeric ability of opto-α-syn proteins, the Cry2 PHR was substituted to Cry2clust domain which induces protein-protein interaction more efficiently than wild-type of Cry2 PHR. N-terminal or C-terminal Cry2clust-tagged opto-α-syn constructs were designed with controls and it was confirmed that α-syn construct fused with Cry2clust C-terminally induces α-syn aggregates efficiently in both of SH-SY5Y cells and PD hiPSC-derived neural progenitor cells (NPCs) (FIG. 4A). Using SNCA triplication hiPSCs, an α-syn-mCherry fused with Cry2clust C-terminally (named opto-α-syn, AAVS1 . . . opto-α-syn)- or an mCherry fused with Cry2clust (named opto-mock, AAVS1 . . . opto-mock)-expressing PD hiPSC line were then generated through CRISPR/Cas9-mediated homologous recombination to fluorescently monitor the optogenetic control of α-syn aggregation (FIGS. 5A and 4B). To examine the optical induction of α-syn aggregation and its effects on mDA neurons, opto-mock or opto-α-syn PD hiPSCs were first differentiated into mDA neurons as described previously (FIG. 5B). There was no detectable difference in the yield of mDA neurons between opto-α-syn and opto-mock PD hiPSCs (FIG. 6A). To investigate whether Cry2clust-based OASIS can optically induce α-syn aggregation in PD hiPSC-derived mDA neurons, live-cell imaging with confocal microscopy was performed. A significant induction of α-syn aggregation in response to blue light illumination only in opto-α-syn-expressing PD hiPSC-derived mDA (opto-α-syn-mDA) neurons was found, but not in opto-mock-expressing mDA (opto-mock-mDA) neurons. It was also found that the number of α-syn aggregates was significantly increased upon blue light illumination in a time-dependent manner in opto-α-syn-mDA neurons, but not in opto-mock-mDA neurons (FIG. 5C), and the α-syn aggregation was spatially regulated. In addition, opto-α-syn-containing aggregates were formed more rapidly in neurite region compared to those in cell body region (FIG. 5D).

Next, whether the optically induced aggregates contain the important markers for PD-associated α-syn aggregates was tested. The optically derived α-syn aggregates were immunostained with 5G4 antibody in TH⁺ opto-α-syn-mDA neurons, and the 5G4⁺ aggregates were also stained with anti-pS129 antibodies in both of neurite and cell body regions. Furthermore, the number of total- or phosphorylated-α-syn-aggregates was significantly increased in opto-α-syn-mDA neurons upon blue light stimulation compared to opto-mock-mDA neurons (FIGS. 5E and 5F). Consistently, the opto-mock-mDA neurons did not show any of light-inducible 5G4⁺ or pS129⁺ aggregates, despite prolonged blue light illumination; demonstrating that this pathogenic aggregate formation was not caused by the light itself.

The expression of HA-opto-mock or HA-opto-α-syn in SH-SY5Y neuronal cells was evaluated with or without illumination. Mock- and HA-opto-α-syn PD-iPSCs-derived mDA neurons with or without blue light stimulation were images; and global transcriptome analyses (RNA-seq) of the indicated conditions was assessed. Scatter plots of all expressed genes in each pairwise (dots, P<0.05 with Benjamini-Hochberg multiple testing correction).

AAVS1::opto-mock or AAVS1::opto-α-syn PD hiPSCs were generated, as schematically represented in FIG. 4A, illustrating the various protein constructs including opto-mock, mCherry-α-syn, N-opto-α-syn, and C-opto-α-syn. Opto-mock, N-opto-α-syn, and C-opto-α-syn have Cry2Clst domain for blue light-induced protein interaction. SH-SY5Y cells were transfected with each construct as indicated for 24 h in dark and then kept in dark or exposed to blue light (34 μW/mm² at 470 nm, 0.5 Hz, 0.5 s) for 21.5 h, followed by immunostaining with 5G4 antibody. PD hiPSC-derived NPCs were transfected with opto-mock or C-opto-α-syn for 24 h in dark and then kept in dark or exposed to blue light (34 μW/mm² at 470 nm, 0.5 Hz, 0.5 s) for 17.5 h, followed by immunostaining with 5G4 antibody. Genomic DNA PCR of AAVS1::opto-mock or AAVS1::opto-α-syn PD hiPSCs. Flanking regions of gRNA-binding site on AAVS1 locus were amplified using following primers: forward, 5′-CTGCCGTCTCTCTCCTGAGT-3′ (SEQ ID NO:4); reverse, 5′-GTGGGCTTGTACTCGGTCAT-3′ (SEQ ID NO:5). Detection of a 1,033 bp fragment is an indicative of insertion into the AAVS1 locus. Non-integrated AAVS1 allele was amplified by using specific primers: forward, 5′-TTCGGGTCACCTCTCACTCC-3′ (SEQ ID NO:6); reverse, 5′-GGCTCCATCGTAAGCAAACC-3′ (SEQ ID NO:7). An untargeted AAVS1 allele produces an ˜500 bp fragment. Scale bars, 10 μm (FIG. 4B).

Disease-associated α-syn aggregation was light-induced. Different AAVS1 locus were targeted using homologous recombination enhanced by CRISPR/Cas9 system in PD hiPSCs (FIG. 5A). Opto-mock or opto-α-syn expressing PD hiPSCs were differentiated into mDA neurons (FIG. 5B). After differentiation, these mDA neurons were exposed to the blue light. PD hiPSCs-derived mDA neurons expressing opto-mock or opto-α-syn were exposed to acute pulsed blue light stimulation (1.5 μW at 488 nm, 0.17 Hz, 1 s) for checking the formation of light-induced aggregates. Representative images of mDA neurons expressing opto-mock (top) or opto-α-syn (bottom) in dark or exposed to blue light were taken, the total area of aggregate in mDA neurons expressing opto-mock or opto-α-syn (FIG. 5C), or in cell body or neurite of opto-α-syn-expressing mDA neurons (FIG. 5D) were quantified over time using automated live-imaging. Error bars represent mean±SEM. Ordinary two-way ANOVA (n=3, each experiment contains at least 40 cells). Opto-mock-expressing (opto-mock-mDA) or opto-α-syn-expressing PD hiPSC-derived mDA (opto-α-syn-mDA) neurons were in dark or exposed to blue light (34 μW/mm² at 470 nm, 0.17 Hz, 0.5 s) for 7 days and then immunostained with 5G4, anti-mCherry and anti-TH antibodies. The mCherry⁺ α-syn aggregates were colocalized with 5G4 in TH⁺ mDA neurons, which is indicated by arrowheads. Opto-α-syn-mDA neurons were in dark or exposed to blue light (34 μW/mm² at 470 nm, 0.17 Hz, 0.5 s) for 7 days and then immunostained with pS129, 5G4, and anti-mCherry antibodies. Arrowheads indicate the co-localized aggregates with three indicated antibodies. The number of 5G4⁺ or pS129⁺ α-syn aggregates in opto-mock- or opto-α-syn-expressing mDA neurons with or without blue light illumination were quantified (FIGS. 5E and 5F). Error bars represent mean±SD. One-way ANOVA followed by Tukey's post hoc test (n=8 for H, 4 images each from 2 independent experiments: 6 for 1, 3 images each from 2 independent experiments). Error bars represent mean±SEM. n.s., not significant. ****P<0.0001. Scale bars, 10 μm.

As detailed in FIGS. 6A-6B neural differentiation into TH⁺ mDA neurons from AAVS1::opto-mock or AAVS1::opto-α-syn PD hiPSCs was assessed. Neural differentiation into mDA neurons from opto-mock or opto-α-syn PD hiPSC's observed in brightfield microscopic images and immunostaining images with anti-TH antibody show that mDA neurons were successfully generated from opto-mock or opto-α-syn expressing PD hiPSCs. TH⁺ mDA neurons expressing opto-mock or opto-α-syn were quantified (FIG. 6A). Error bars represent mean±SD. Student's t-test (n=36, 6 images each from six independent experiments). Opto-mock- or opto-α-syn-mDA neurons were exposed to acute blue light stimulation (1.5 μW at 488 nm, 0.17 Hz, 1 s) using live-cell imaging. Representative images of mDA neurons expressing opto-mock (top) or opto-α-syn (bottom), or cell body (top) or neurite (bottom) of opto-α-syn-mDA neurons, exposed by blue light. Opto-mock mDA neurons did not show any pS129⁺ or 5G4⁺ α-syn aggregates even in the blue light stimulation. Cells were in dark or exposed to blue light (34 μW/mm² at 470 nm, 0.17 Hz, 0.5 s) for 7 days and then immunostained with pS129, 5G4, and anti-mCherry antibodies. Opto-α-syn mDA neurons were exposed to blue light (34 μW/mm² at 470 nm, 0.17 Hz, 0.5 s) for 7 days and then immunostained with the indicated antibodies. White arrowheads indicate the co-localized aggregates with pS129, 504, and mCherry or Syn303, EP1536Y, and mCherry signals; white arrows indicate the co-localized aggregates with pS129 and 504 or Syn303 and EP1536Y signals. Opto-α-syn-mDA neurons were in dark for 7 days and then immunostained with the indicated antibodies. Quantification of relative levels of cell number to control was evaluated. Opto-mock or Opto-α-syn mDA neurons were in dark or exposed to blue light (34 μW/mm² at 470 nm, 0.17 Hz, 0.5 s) for 7 days (FIG. 6B). Error bars represent mean±SD. One-way ANOVA followed by Tukey's post hoc test (n=30, 6 images each from 5 independent experiments).

### Example 4

### Pathogenic Effects of Light-Induced α-Syn Aggregation on mDA Neurons

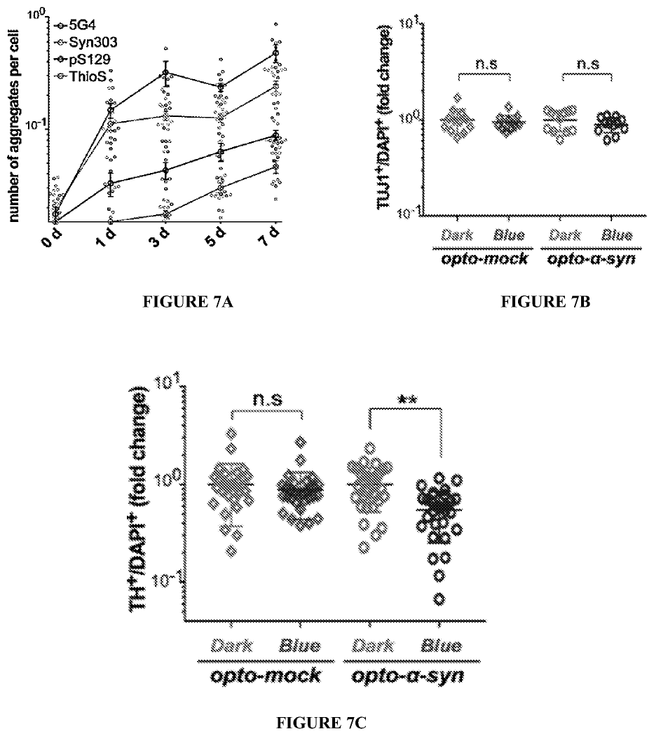

Additional investigation indicated that the light-induced α-syn aggregates were co-stained with p62 and ubiquitin, which are considered as one of the markers for pathogenic α-syn aggregates, together with Syn303 and another anti-pS129 (EP1536Y). More importantly, the optically induced α-syn aggregates were stained with ThioS, which is a marker for the beta-sheet-containing amyloid. As expected, any of these pathogenic markers-positive α-syn aggregates was not formed in dark condition. Furthermore, the changes in the key pathogenic markers-positive aggregates were examined over time of blue light illumination (FIG. 7A). While Syn303⁺ or 5G4⁺ α-syn aggregates were rapidly increased with blue light stimulation, the phosphorylated or ThioS⁺ α-syn aggregates were slowly increased comparatively; suggesting these conformation-selective antibodies and probe might be related to the gradual progress of pathological α-syn aggregation (FIG. 7A). In summary, the OASIS can generate the controllable blue light-dependent pathogenic α-syn aggregates in PD hiPSC-derived mDA neurons.

Next, the effects of optically induced α-syn aggregation were investigated on mDA neurons. To determine whether these α-syn aggregates are toxic to mDA neurons, TH⁺ mDA neurons with or without blue light illumination were quantified. Although the blue light illumination did not induce a significant change in the number of total neurons (stained with TUJ1) or total cells (stained with DAPI) (FIGS. 7B and 7B), a significant loss of TH⁺ neurons in opto-α-syn-mDA neurons after blue light illumination was detected, but not in opto-mock-mDA neurons (FIG. 7C). Collectively, these data suggested that blue light stimulation on opto-α-syn-mDA neurons could induce the pathological α-syn aggregate formation, which shows neurotoxicity to TH⁺ mDA neurons.

As illustrated in FIGS. 7A-7C, selective death of PD hiPSC-derived mDA neurons was induced by the optogenetic α-syn aggregation system. Opto-α-syn-mDA neurons were exposed to blue light (34 μW/mm² at 470 nm, 0.17 Hz, 0.5 s) for 7 days and then immunostained with Syn303, EPI536Y, and anti-mCherry antibodies or anti-ubiquitin, p62, and mCherry antibodies. Arrowheads indicate the co-localized aggregates with three indicated antibodies. Opto-α-syn-mDA neurons were exposed to blue light (34 μW/mm² at 470 nm, 0.17 Hz, 0.5 s) for 7 days and then stained with Thioflavin S. Arrowheads indicate the co-localized aggregates with ThioS and mCherry signal. Opto-α-syn-mDA neurons were in dark or exposed to blue light (34 μW/mm² at 470 nm, 0.17 Hz, 0.5 s) for the indicated time and then immunostained with Syn303. EP1536Y, and 5G4 or stained with ThioS (FIG. 7A). Error bars represent mean±SEM. Ordinary two-way ANOVA (n=12, 6 images each from two independent experiments). Opto-mock- or opto-α-syn-mDA neurons were in dark or exposed to blue light (34 μW/mm² at 470 nm, 0.17 Hz, 0.5 s) for 7 days. These cells were immunostained with anti-TUJ1 antibody and then subjected to quantification of the TUJ1⁺ area per DAPI (FIG. 7B). Error bars represent mean±SEM. One-way ANOVA followed by Tukey's post hoc test (n=12, 6 images each from 2 independent experiments). Opto-mock- or opto-α-syn-mDA neurons were in dark or exposed to blue light (34 μW/mm² at 470 nm, 0.17 Hz, 0.5 s) for 7 days. These cells were immunostained with anti-TH antibody and then subjected to quantification of the TH⁺ area normalized to DAPI (FIG. 7C). Error bars represent mean±SD. One-way ANOVA followed by Tukey's post hoc test (n=30, 6 images each from 5 independent experiments). Error bars represent mean±SEM. n.s., not significant. **P<0.01.

### Example 5

### Application of the Optical Induction System for α-Syn Aggregation into High-Content Imaging Compound Screening

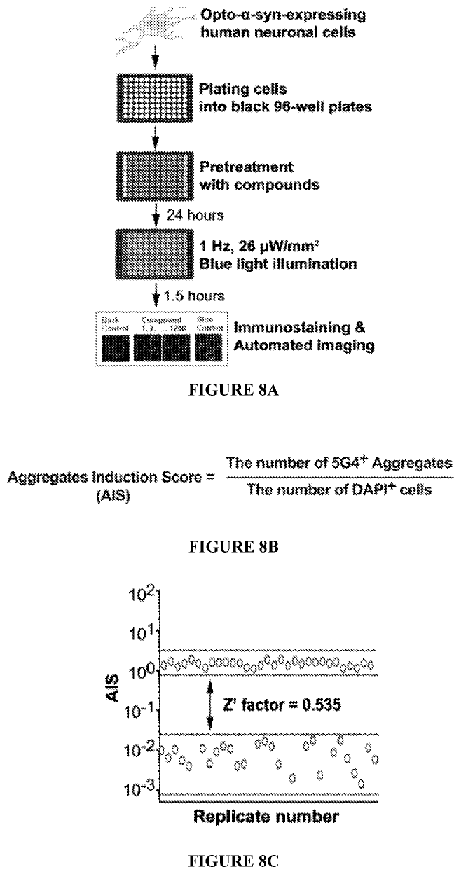

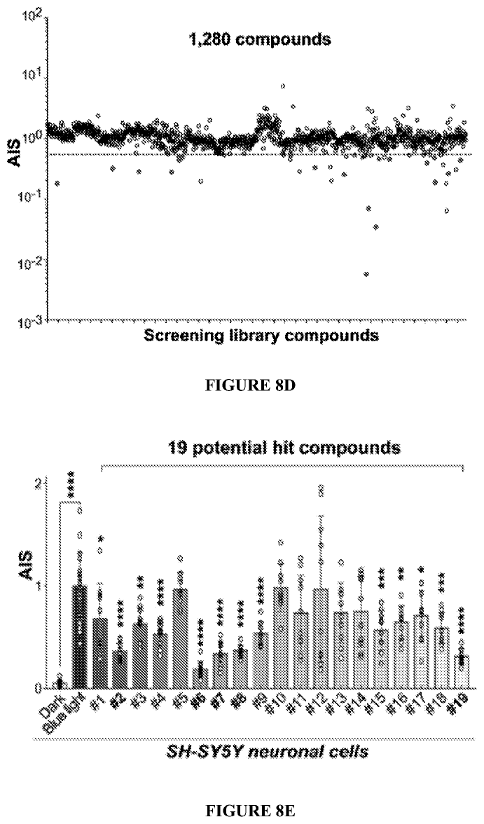

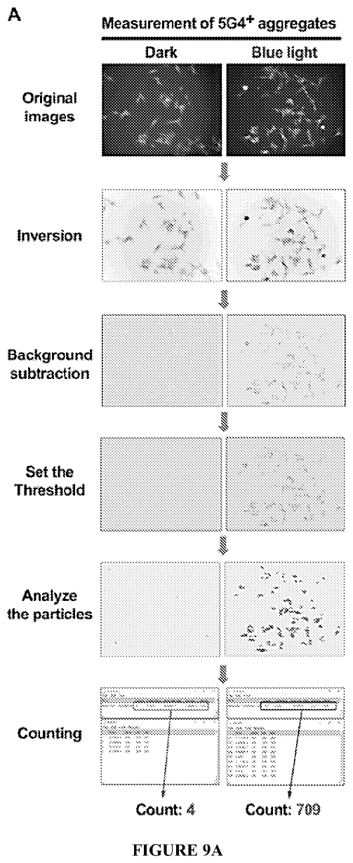

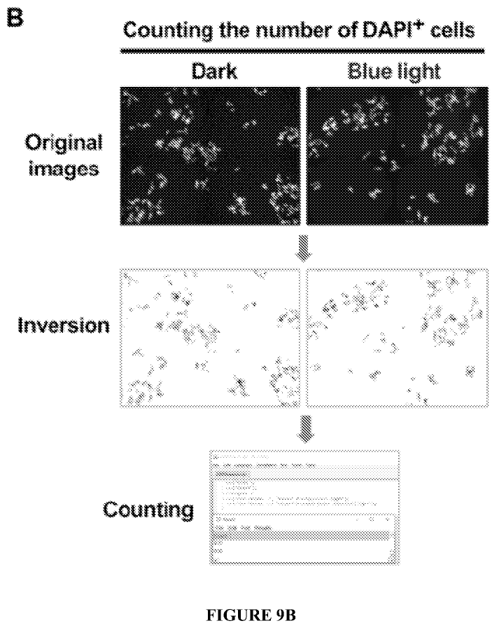

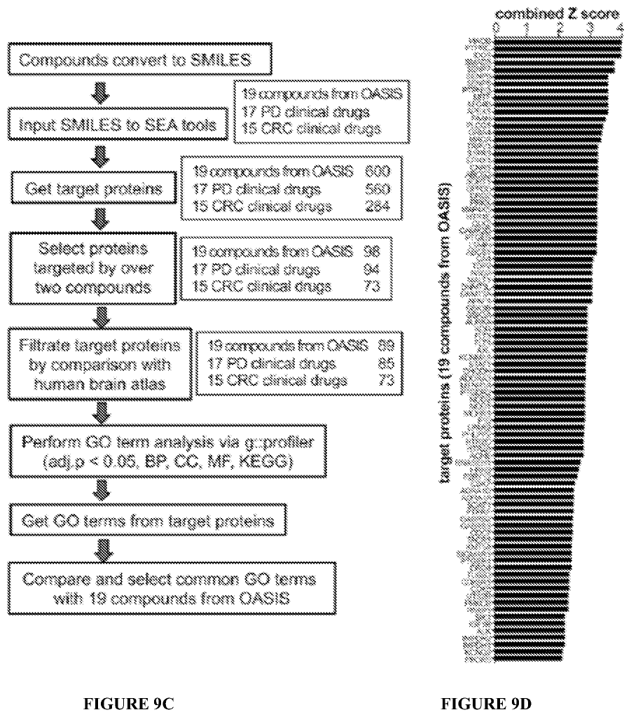

There have been strong needs to develop a proper human neuronal cell-based in vitro screening platform to identify novel compounds for curing PD. However, only few studies have set up cell-based assays due to a lack of technology that could induce and control pathological protein aggregations. To identify chemical compounds that inhibit or delay early-stage aggregations of α-syn, OASIS with high-content imaging (HCI) assay using 5G4 antibody to measure the amount of formation of aggregated α-syn was developed. conditions of cell plating, immunocytochemistry with 5G4 antibody, and automated imaging systems for 96-wells were optimized (FIG. 8A). To process the aggregations of final images, image analysis method using ImageJ software were utilized and the new measurement of the number of 5G4⁺ aggregates, termed as Aggregates Induction Score (AIS) was developed (FIGS. 8B, 9A, and 9B). To validate OASIS-based HCI assay, a control study with or without blue light illumination (as a negative or positive control) was performed on 96-well-plate format, demonstrating excellent Z′ values of 0.535 (FIGURE SC). Next, a library of 1,280 small molecules, which contain diverse high-purity compounds active at GPCRs, kinases, ion channels, nuclear receptors, and transporters was screened. Compounds were screened at 1 μM in 0.1% DMSO with each plate containing 0.1% DMSO control wells (FIG. 8D). Through the primary screening with calculating AIS, we selected 19 compounds (hit rate, 1.5%) as potential inhibitors of the early-stage α-syn aggregation (closed circles; FIG. 8D). Among them, 4 of 19 compounds have been previously reported as potential neuroprotective drugs for PD, confirming the feasibility of the OASIS-based HCI assay. Further validation with those potential hits were performed. To confirm the reproducibility of inhibitory effects of those compounds, AIS from two independent experiments were measured following blue light stimulation under standard 24-well culture conditions; especially, 5 potential hit compounds showed a significant decrease of AIS compared to blue light-illuminated DMSO control: ‘BVT 948’ (BVT; #2 in FIG. 8E), ‘C 021 dihydrochloride’ (CDC; #6 in FIG. 8E), ‘BAG 956’ (BAG; #7 in FIGURE SE), ‘Arcyriaflavin A’ (AFA; #8 in FIGURE SE), and ‘AZD 1480’ (AZD; #19 in FIGURE SE) (FIG. 8E; the detailed information of numbered compounds used in FIG. 8E was described in Table 1).

TABLE 1
List of 19 compounds screened by the optical
induction system for α-syn aggregation.

| No. Compound | | Fanction | AIS ⁽¹⁾ | p-value | Note |
|---|---|---|---|---|---|
| 1 | Cyclothiazide | AMPA selective desensitization | 0.68 | 2.00E−02 | * |
| | | inhibitor | | | |
| 2 | BVT 948 | Non-competitive protein tyrosine | 0.36 | 2.00E−06 | **** |
| | | phosphatase inhibitor | | | |
| 3 | C1976 | Selective ACAT inhibitor | 0.63 | 2.00E−03 | ** |
| 4 | NE 100 | Selective sigmal antagonist | 0.53 | 1.00E−04 | *** |
| | hydrochloride | | | | |
| 5 | BX 471 | Potent, selective CCR1 antagonist | 0.97 | 8.00E−01 | |
| 6 | C 021 | Potent CCR4 antagonist | 0.19 | 0.00E+00 | **** |
| | dihydrochloride | | | | |
| 7 | BAG 956 | Dual PI 3-kinase and PDPK1 (PDK1) | 0.34 | 1.00E−06 | **** |
| | | inhibitor | | | |
| 8 | Arcyriaflavin A | Potent cdk4/cyclin D1 and CaM Kinase | 0.37 | 2.00E−06 | **** |
| | | II inhibitor; Antiviral agent | | | |
| 9 | Amlexanox | Inhibitor of TBK1 and IKKepsilon; | 0.53 | 2.00E−04 | *** |
| | | antiallergic agent | | | |
| 10 | Kartogenin | Potently induces chondrogenesis | 0.98 | 9.00E−01 | |
| | | in MSCs | | | |
| 11 | Neuropathiazol | Selective inducer of neuronal | 0.73 | 5.00E−02 | |
| | | differentiation in hippocampal | | | |
| | | neural progenitors | | | |
| 12 | Napabucasin | STAT3 inhibitor; also blocks cancer | 0.97 | 9.00E−01 | |
| | | stem cell self-renewal | | | |
| 13 | Dexamethasone | Anti-inflammatory glucocorticoid | 0.74 | 4.00E−02 | * |
| 14 | XD 14 | BET bromodomain inhibitor | 0.75 | 6.00E−02 | |
| 15 | Mycophenolic | Inosine monophosphate dehydrogenase | 0.57 | 6.00E−04 | *** |
| | acid | inhibitor | | | |
| 16 | Cilostazol | PDE3A inhibitor. Also, adenosine | 0.65 | 3.00E−03 | ** |
| | | uptake inhibitor | | | |
| 17 | Mycophenolate | Inosine monophosphate dehydrogenase | 0.71 | 2.00E−02 | * |
| | mofetil | inhibitor | | | |
| 18 | Rizatriptan | Potent 5-HT1 B/1D agonist | 0.59 | 8.00E−04 | *** |
| | benzoate | | | | |
| 19 | AZD 1480 | Potent and selective JAK2 inhibitor; | 0.31 | 0.00E+00 | **** |
| | | antiangiogenic | | | |

⁽¹⁾ AIS, aggregates induction score

As illustrated in FIGS. 8A-8E, high-content imaging screening with the optogenetic α-syn aggregation system was performed, following the schematic representation of the process of high-content imaging (HCI) screening with optogenetics-assisted method of alpha-synuclein aggregation induction system (OASIS) (FIG. 8A). SH-SY5Y cells expressing opto-α-syn were in dark or exposed to blue light (26 μW/mm² at 470 nm, 1 Hz, 1 s) for 1.5 h under 96-well plate culture conditions, and then immunostained with 5G4 antibody. The equation of Aggregates Induction Score (AIS) (FIG. 8B) was used for the calculation of Z′-factor for HCI screening with OASIS (FIG. 8C). Dots represent wells with the following treatment: opto-α-syn cells in dark (lower circles) or exposed to blue light (upper circles). Arrow represents the degree of separation (Z′-factor) between light-illuminated and darkness controls. A scatter plot of compounds screened in the OASIS-based HCI assay was generated, where for each compound, the corresponding AIS (y-axis, logo scale) observed in the drug-treated human neuroblastoma cells is plotted (positive control was set as 1.0). The 1,280 compounds were screened and are shown on the x-axis. Closed circles represent 19 selected potential hit compounds (FIG. 8D). Effect of treatment with 19 compounds were validated on α-syn aggregation in HA-opto-α-syn SH-SY5Y neuronal cells under 24-well plate culture conditions (FIG. 8E). Detailed information of numbered compounds was described in Table 1. One-way ANOVA followed by Dunnett's post hoc test (n=10 or 20, 5 or 10 images each from two independent experiments). *P<0.05, **P<0.01, ***P<0.001, ****P<0.0001.

As illustrated in FIGS. 9A-9B, high-content imaging screening with the optogenetic α-syn aggregation system was performed. The measurement of 5G4⁺ aggregates in opto-mock or opto-α-syn SH-SY5Y neuronal cells was assessed. Cells were in dark or exposed to blue light (26 μW/mm² at 470 nm, 1 Hz, 1 s) for 1.5 h, and then immunostained with 5G4 antibody. After staining, four images per well were automatically captured by using BD Pathway™ 855 Bioimager and analyzed with ImageJ software. Images were converted to grayscale, and a threshold was adjusted to exclude the background based on the size of particles. Once the value of threshold was determined, all the images were applied with the fixed threshold, and then the number of aggregates was measured (FIG. 9A). Number of DAPI from the original images were counted (FIG. 9B). Representative images of opto-α-syn SH-SY5Y neuronal cells treated with 19 compounds under 24-well plate culture conditions were imaged. SH-SY5Y cells expressing opto-α-syn were in dark or exposed to blue light (34 μW/mm² at 470 nm, 1 Hz, 0.5 s) for 1.5 h, and then immunostained with 5G4 antibody.

### Example 6

### Comparative Chemogenomic Analysis of Potential Hit Compounds from Oasis-Based HCI Assay and PD Clinical Drugs

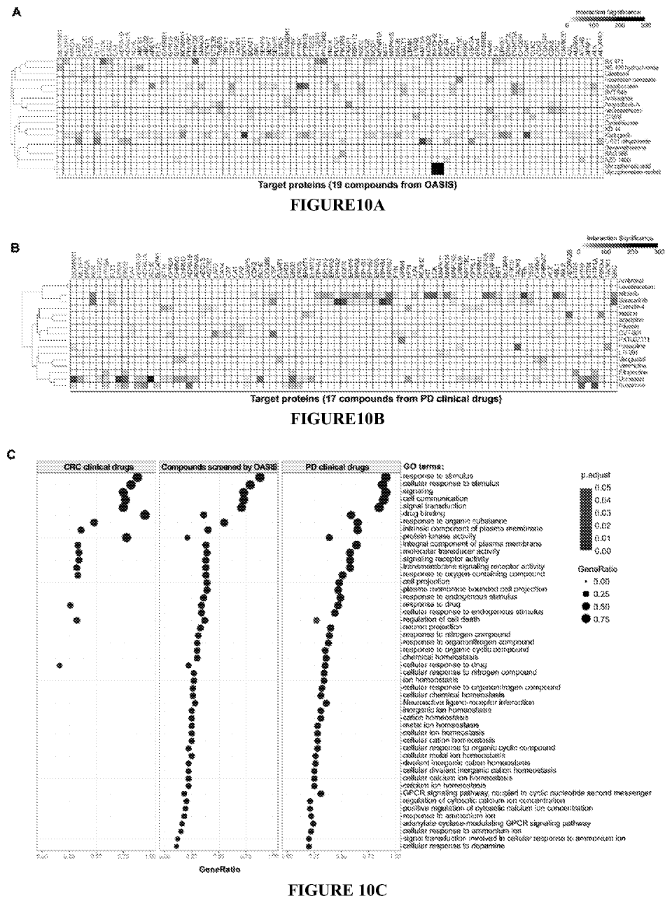

Chemogenomic analysis of 19 potential hit compounds selected by the OASIS-based HCI assays (named OASIS compounds) was further performed. First isometric simplified molecular-input line-entry system (SMILES) of each compound was obtained and put into the similarity ensemble approach (SEA) computational tool to predict drug-protein interactions (FIG. 9C). The SEA-predicted drug-protein pairs were filtered by predicted interaction p-values<0.05 and selected human proteins, which yielded 600 target proteins from 19 OASIS compounds. To identify the common targets or pathways, 98 proteins targeted commonly by over two compounds were selected and it was confirmed that 89 out of 98 proteins were expressed in human brain by filtering the target list through the human protein atlas (HPA) database. Next, combined z-scores were calculated from these 89 target proteins by adding normalized z-scores from primary screening (FIG. 9D). Based on these values, compound-protein interaction was mapped, and it showed a significant interaction between 19 OASIS compounds and target proteins (FIG. 10A). To find out which biological pathways are associated with reducing α-syn aggregation, Gene Ontology (GO) term enrichment analysis was performed with 89 target proteins. Interestingly, various PD-related GO terms were obtained such as ion homeostasis, neuroactive ligand-receptor interaction, and cellular response to dopamine (Tables 2-4). To validate these results, the chemogenomic analysis method was applied to PD and colorectal cancer (CRC) clinical drugs as a positive and negative control, respectively. Notably, drug-protein interaction heatmap of PD clinical drugs revealed 13 common target proteins with compounds screened by OASIS, while CRC clinical drugs showed only 3 common target proteins, as well as showed strong interaction of 17 compounds with its target proteins (FIG. 10B). Furthermore, GO term analysis of PD clinical drugs indicated that 281 out of 322 GO terms obtained from OASIS compounds are common with GO terms from PD clinical drugs. These 281 GO terms showed high correlation between PD clinical drugs and OASIS compounds (adj. p-value, Spearman's correlation coefficients (r)=0.5130, P<0.0001; GeneRatio, r=0.9376, P<0.0001); however, there was no significant correlation between the GO terms from CRC clinical drugs and OASIS compounds (adj. p-value, r=0.0355, P=0.5532; GeneRatio, r=0.7797, P<0.0001). Consistently, comparative GO enrichment dot plots with top 50 tanked GO terms revealed that dot plot of OASIS compounds is similar to the dot plot of PD clinical drugs compared to the plot of CRC clinical drugs (FIG. 5C). Taken together, these results not only validated the feasibility of OASIS-based HCI assay by comparing them with the results of PD clinical drugs but also suggested that OASIS-mediated screened compounds could target PD-related pathways.

TABLE 2
List of 19 compounds screened by OASIS.

| Compound | SMILES | note |
|---|---|---|
| Cyclothiazide | NS(═O)(═O)C1═C(C1)C═C2NC(NS | A newly identified |
| | (═O)(═O)C2═C1)C1CC2CC1C═C2 | compound inhibiting |
| | | α-syn aggregation |
| BVT 948 | CC1(C)C(═O)N═C2C1═C(O)C(═O) | A newly identified |
| | C1═CC═CC═C21 | compound inhibiting |
| | | α-syn aggregation |
| C1 976 | CCCCCCCCCCC(C)(C)C(═O)NC1═C | A newly identified |
| | (OC)C═C(OC)C═C1OC | compound inhibiting |
| | | α-syn aggregation |
| NE 100 | CCCN(CCC)CCC1═CC═C(OC)C | A newly identified |
| hydrochloride | (OCCC2═CC═CC═C2)═C1•Cl | compound inhibiting |
| | | α-syn aggregation |
| BX 471 | FC1═CC═C(CN2C[C@@H] | A newly identified |
| | (C)N(C(COC3═CC═C(C1)C═C3NC | compound inhibiting |
| | (N)═O)═O)CC2)C═C1 | α-syn aggregation |
| C 021 | COC1═C(OC)C═C(N═C(N4CCC(N5CCCCC5) | A newly identified |
| dihychloride | CC4)N═C2NC3CCCCCC3)C2═C1•C1•C1 | compound inhibiting |
| | | α-syn aggregation |
| BAG 956 | CC3═NC1═C(N3C4═CC═C(C(C) | A newly identified |
| | (C#N)C)C═C4)C2═C(C═CC | compound inhibiting |
| | (C#CC5═CN═CC═C5)═C2)N═C1 | α-syn aggregation |
| Arcyriaflavin A | O═C1NC(═O)C2═C1C1═C | A newly identified |
| | (NC3═CC═CC═C13)C1═C2C2═ | compound inhibiting |
| | C(N1)C═CC═C2 | α-syn aggregation |
| Amlexanox | CC(C)C1═CC═C2C(C(C(C═C(C(O)═O) | TLR2 signaling- |
| | C(N)═N3)═C3O2)═O)═C1 | mediated decrease of |
| | | α-syn pathology in |
| | | neuronal cells |
| Kartogenin | O═C(NC2═CC═C(C3═CC═CC═C3) | A newly identified |
| | C═C2)C1═CC═CC═C1C(O)═O | compound inhibiting |
| | | α-syn aggregation |
| Neuropathiazol | O═C(OCC)C1═CC═C(N(C2═CSC | A newly identified |
| | (C3═CC═CC═C3)═N2)C)C═C1 | compound inhibiting |
| | | α-syn aggregation |
| Napabucasin | O═C(C1═C2C═C(C(C)═O)O1) | A newly identified |
| | C3═CC═CC═C3C2═O | compound inhibiting |
| | | α-syn aggregation |
| Dexamethasone | O═C1C═CC2(C)C(CC[C@]3([H]) | inhibition of copper- |
| | [C@@](F)2[C@@H](O)C[C@@] | induced alpha- |
| | 4(C)[C@]([H])3C[C@@H](C) | synuclein aggregation |
| | [C@@](O)4C(CO)═O)═C1 | by a metallothionein- |
| | | dependent mechanism |
| XD 14 | CC1═C(C(C)═O)C(CC)═C(C(NC2═CC | A newly identified |
| | (S(N(CC)CC)(═O)═O)═CC═C2O)═O)N1 | compound inhibiting |
| | | α-syn aggregation |
| Mycophenolic | COC1═C(C)C2═C(C(═O)OC2)C | A newly identified |
| acid | (O)═C1C\C═C(/C)CCC(O)═O | compound inhibiting |
| | | α-syn aggregation |
| Cilostazol | O═C1CCC2═CC | the neuroprotective |
| | (OCCCCC3═NN═NN3C3CCCCC3)═CC═C2N1 | effect in rotenone- |
| | | induced PD model in |
| | | rats |
| Mycophenolate | O═C(OC2)C1═C2C(C)═C(OC)C | A newly identified |
| mofetil | (C/C═C(C)/CCC(OCCN3CCOCC3)═ | compound inhibiting |
| | O)═C1O | α-syn aggregation |
| Rizatriptan | CN(CCC2═CNC1═CC═C | A newly identified |
| benzoate | (CN3N═CN═C3)C═C12)C•O═C | compound inhibiting |
| | (C4═CC═CC═C4)O | α-syn aggregation |
| AZD 1480 | C[C@@H](C1═NC═C | Protection of |
| | (F)C═N1)NC2═NC═C(C1)C | α-Synuclein-Induced |
| | (NC3═NNC(C)═C3)═N2 | Neuroinflammation |
| | | by Inhibition of the |
| | | JAK/STAT Pathway |

TABLE 3
List of 17 PD clinical trial drugs

| | | Clinical |
|---|---|---|
| Compound | SMILES | phase |
| Ambroxol | C1CC(CCC1NCC2═C(C(═CC(═C2)Br)Br)N)O | Phase 2 |
| Exendin-4 | [H]/N═C(\N)/NCCC[C@@H](C(═O)N[C@@H] | Phase 2 |
| | (CC(C)C)C(═O)N[C@@H](Cc1ccccc1)C(═O)N[C@@H] | |
| | ([C@@H](C)CC)C(═O)N[C@@H](CCC(═O)O)C(═O) | |
| | N[C@@H](Cc2c[nH]c3c2cccc3)C(═O)N[C@@H] | |
| | (CC(C)C)C(═O)N[C@@H](CCCCN)C(═O)N[C@@H] | |
| | (CC(═O)N)C(═O)NCC(═O)NCC(═O)N4CCC[C@H]4C(═O) | |
| | N[C@@H](CO)C(═O)N[C@@H](CO)C(═O)NCC(═O) | |
| | N[C@@H](C)C(═O)N5CCC[C@H]5C(═O)N6CCC[C@H]6 | |
| | C(═O)N7CCC[C@H]7C(═O)N[C@@H](CO)C(═O)N)NC | |
| | (═O[C@H](C(C)C)NC(═O)[C@H](C)NC(═O)[C@H] | |
| | (CCC(═O)O)NC(═O)[C@H](CCC(═O)O)NC(═O)[C@H] | |
| | (CCC(═O)O)NC(═O)[C@H](CCSC)NC(═O)[C@H](CCC(═O) | |
| | N)NC(═O)[C@H](CCCCN)NC(═O)[C@H](CO)NC(═O) | |
| | [C@H](CC(C)C)NC(═O)[C@H](CC(═O)O)NC(═O)[C@H] | |
| | (CO)NC(═O)[C@H]([C@@H](C)O)NC(═O)[C@H] | |
| | (Cc8ccccc8)NC(═O)[C@H]([C@@H](C)O)NC(═O)CNC(═O) | |
| | [C@H](CCC(═O)O)NC(═O)CNC(═O)[C@H](Cc9cnc[nH]9)N | |
| LT1-291 | O═C(C1═C2N═C(C)C═C(C)N2N═C1)N[C@@H]3CC | Phase 2 |
| | [C@@H](OCCCCC)CC3 | |
| Levetiracetam | CC[C@@H](C(═O)N)N1CCCC1═O | Phase 2 |
| Nilotinib | CC1═C(C═C(C═C1)C(═O)NC2═CC(═CC(═C2)C(F)(F)F)N3C═C | Phase 2 |
| | (N═C3)C)NC4═NC═CC(═N4)C5═CN═CC═C5 | |
| Rasagiline | C#CCN[C@@H]1CCC2═CC═CC═C12 | Phase 3 |
| Riluzole | C1═CC2═C(C═C1OC(F)(F)F)SC(═N2)N | Phase 2 |
| Saracatinib | CN1CCN(CC1)CCOC2═CC3═C(C(═C2)OC4CCOCC4)C(═NC═N3) | Phase 1 |
| | NC5═C(C═CC6═C5OCO6)C1 | |
| Venglustat | O═C(O[C@@H]1CN2CCC1CC2)NC(C)(C3═CSC(C4═CC═C(F) | Phase 2 |
| | C═C4)═N3)C | |
| Inosine | C1═NC2═C(C(═O)N1)N═CN2[C@H]3[C@@H] | Phase 3 |
| | ([C@@H]([C@H](O3)CO)O)O | |
| Isradipine | CC1═C(C(C(═C(N1)C)C(═O)OC(C)C)C2═CC═CC3═NON═C32)C | Phase 3 |
| | (═O)OC | |
| PXT002331 | C1COCCN1CCCC2═CC\3═C(C═C2)OC(═C/C3═N\O) | Phase 2 |
| | C4═NC═C5C═CSC5═C4 | |
| CVT-301 | C1═CC(═C(C═C1C[C@@H](C(═O)O)N)O)O | FDA |
| | | approved |
| Eltoprazine | C1CN(CCN1)C2═C3C(═CC═C2)OCCO3 | Phase 2 |
| Donepezil | COC1═C(C═C2C(═C1)CC(C2═O)CC3CCN(CC3)CC4═CC═CC═C4)OC | Phase 4 |
| Varenicline | C1C2CNCC1C3═CC4═NC═CN═C4C═C23 | Phase 2 |
| Buspirone | C1CCC2(C1)CC(═O)N(C(═O)C2)CCCCN3CCN(CC3)C4═NC═CC═N4 | Phase 2 |

TABLE 4
List of 15 CRC clinical trial drugs

| | | Clinical |
|---|---|---|
| Compound | SMILES | phase |
| Camptosar | CCC1═C2CN3C(═CC4═C(C3═O)COC(═O)[C@@]4(CC)O) | FDA |
| (Irinotecan | C2═NC5═C1C═C(C═C5)OC(═O)N6CCC(CC6)N7CCCCC7•C1 | approved |
| Hydrochloride) | | |
| Capecitabine | CCCCCOC(═O)NC1═NC(═O)N(C═C1F)[C@H]2[C@@H] | FDA |
| (Xeloda) | ([C@@H]([C@H](O2)C)O)O | approved |
| Eloxatin | C1CC[C@H]([C@@H](C1)N)N•C(═O)(C(═O) | FDA |
| | [O—])[O—]•[Pt+2] | approved |
| 5-FU | C1═C(C(═O)NC(═O)N1)F | FDA |
| | | approved |
| Fusilev | C1C(N(C2═C(N1)N═C(NC2═O)N)C═O)CNC3═CC═C(C═C3)C(═O) | FDA |
| | N[C@@H](CCC(═O)[O—])C(═O)[O—]•[Ca+2] | approved |
| Lonsurf | C1CC(═N)N(C1)CC2═C(C(═O)NC(═O)N2)C1•C1[C@@H] | FDA |
| | ([C@H](O[C@H]1N2C═C(C(═O)NC2═O)C(F)(F)F)CO)O•C1 | approved |
| Oxaliplatin | C1CC[C@H]([C@@H](C1)[NH—])[NH—]•C | FDA |
| | (═O)(C(═O)[O—])[O—]•[Pt+4] | approved |
| Stivarga | CNC(═O)C1═NC═CC(═C1)OC2═CC(═C(C═C2)NC(═O)NC3═CC(═C | FDA |
| (Regorafenib) | (C═C3)C1)C(F)(F)F)F | approved |
| encorafenib | C[C@@H](CNC1═NC═CC(═N1)C2═CN(N═C2C3═C(C(═CC | FDA |
| (braftovi) | (═C3)C1)NS(═O)(═O)C)F)C(C)C)NC(═O)OC | approved |
| Khapzory | C1[C@@H](N(C2═C(N1)N═C(NC2═O)N)C═O)CNC3═CC═C(C═C3)C | FDA |
| | (═O)N[C@@H](CCC(═O)[O—])C(═O) | approved |
| | [O—]•[Na+]•[Na+] | |
| Vemurafenib | CCCS(═O)(═O)NC1═C(C(═C(C═C1)F)C(═O)C2═CNC3═C2C═C | Phase 2 |
| | (C═N3)C4═CC═C(C═C4)C1)F | |
| Dabrafenib | CC(C)(C)C1═NC(═C(S1)C2═NC(═NC═C2)N)C3═C(C(═CC═C3) | Phase 2 |
| | NS(═O)(═O)C4═C(C═CC═C4F)F)F | |
| trametinib | CC1═C2C(═C(N(C1═O)C)NC3═C(C═C(C═C3)1)F)C(═O)N(C(═O) | Phase 2 |
| | N2C4═CC═CC(═C4)NC(═O)C)C5CC5 | |
| binimetinib | CN1C═NC2═C1C═C(C(═C2F)NC3═C(C═C(C═C3)Br)F)C(═O)NOCCO | Phase 3 |
| lapatinib | CS(═O)(═O)CCNCC1═CC═C(O1)C2═CC3═C(C═C2)N═CN═C3NC4═CC | Phase 2 |
| | (═C(C═C4)OCC5═CC(═CC═C5)F)C1 | |

The main steps of SEA-mediated target analysis were schematically represented in FIG. 9C. Combined Z score of target proteins obtained from 19 compounds screened through OASIS were listed in FIG. 9D.

As shown in FIGS. 10A-10C, potential hit compounds from primary screening were validated through comparative chemogenomic analysis with PD clinical drugs. The drug-protein interaction matrix for the significantly enriched (FIG. 10A) 89 drug target proteins from 19 compounds screened by OASIS, (FIG. 10B) 85 drug target proteins from 17 PD clinical drugs. Shading represents the significance of the predicted interaction based on its z-score. Comparative Gene Ontology (GO) term dot plots from significant target proteins of CRC clinical drugs, compounds screened by OASIS, and PD clinical drugs. Y-axis indicates the GO term and X-axis shows the GeneRatio per GO term. Color gradient and size of dots represent adjusted p-values and GeneRatio, respectively (FIG. 10C).

### Example 7

### Neuroprotective Effect of the Screened Compounds in Opto-α-Syn Expressing PD hiPSC-Derived mDA Neurons

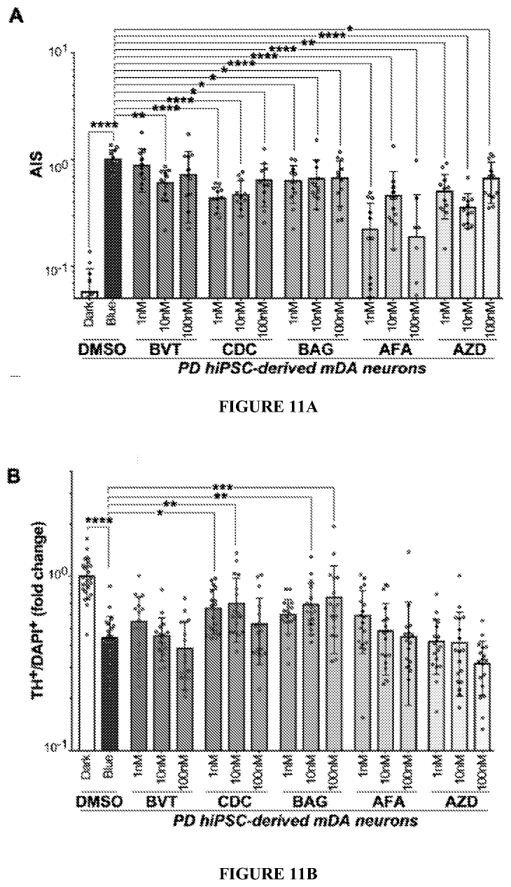

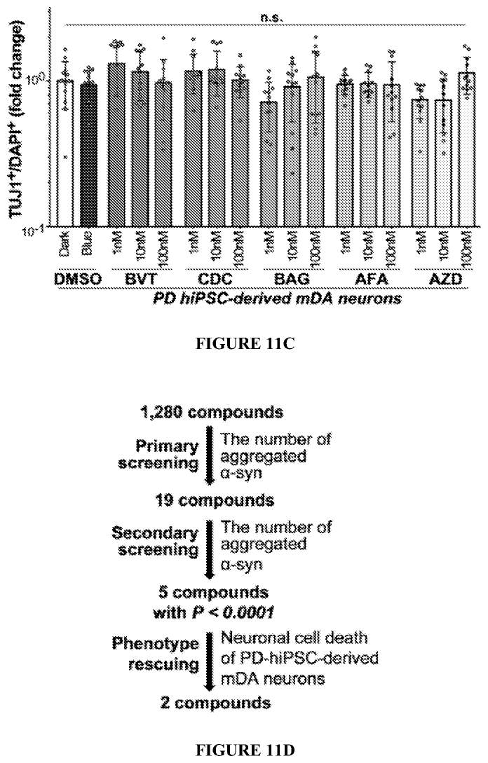

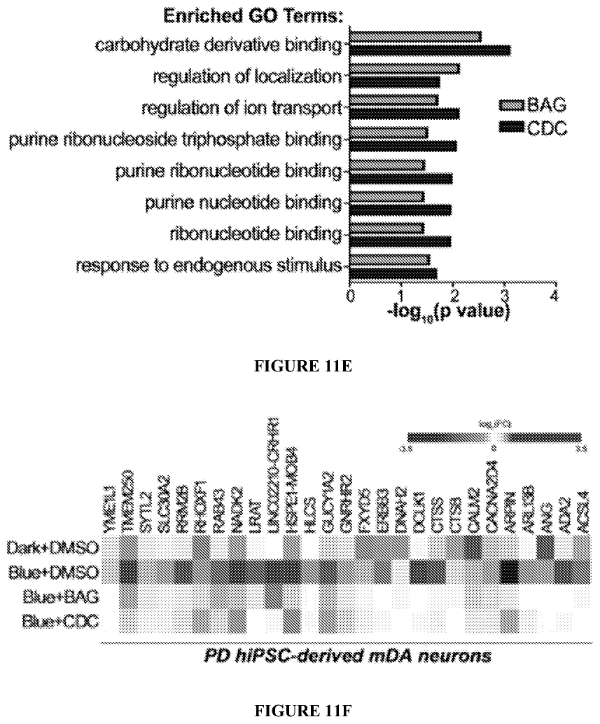

Whether these potential hit compounds can inhibit α-syn aggregation and rescue aggregation-induced neuronal toxicity was tested on PD hiPSC-derived mDA neurons. The top 5 ranked small compounds in FIGURE SE were selected and these compounds to were used to treat opto-α-syn PD hiPSC-derived mDA neurons at three different concentrations (1 nM, 10 nM, 100 nM) with blue light stimulation. Induction of the 5G4⁺ aggregate formation in opto-α-syn PD hiPSC-derived mDA neurons was significantly decreased in response to all five compounds treatment compared to DMSO treatment (FIG. 11A). Furthermore, a significant increase in the survival rate of TH⁺ mDA neurons particularly was observed following the treatment of CDC and BAG compared to DMSO control group (P=0.0282 for 1 nM CDC, 0.0088 for 10 nM CDC, 0.0055 for 10 nM BAG, 0.0001 for 100 nM BAG; FIG. 11B). These compounds did not induce any notable changes to the number of TUJ1⁺ neurons (FIG. 11C). Overall, the two compounds were finally selected among the potential hit drugs from our novel opto-α-syn neuronal cell model-based primary screening (FIG. 11D), and they considerably rescued neuronal cell death from aggregation-induced TH⁺ mDA neuron-selective toxicity in PD-iPSC derived neurons. To identify possible molecules involved in this compound-mediated recovery of TH⁺ mDA neurons, RNA-sequencing (RNA-seq) analysis was performed with four different samples (dark condition with DMSO, blue light stimulation with DMSO, blue light stimulation with BAG treatment, and blue light stimulation with CDC treatment). To find BAG- or CDC-responsive genes, differentially expressed genes (DEGs) of DMSO-treated samples upon blue light stimulation were first extracted, and then the DEGs were filtered with criteria of which became non-DEGs in response to CDC or BAG treatment. Same chemogenomic analysis used in FIGS. 10A-10C was conducted with BAG and CDC to identify predicted targets and related GO terms, and then these GO terms were compared with the GO terms obtained from BAG- or CDC-responsive DEGs in RNA-seq analysis. Notably, not only common potential targets were found, but also significant PD-associated GO terms including regulation of ion transport and purine nucleotide binding were commonly identified in the results of chemogenomic and RNA-seq analysis of BAG or CDC treatment (FIGS. 11E and 11F), suggesting that BAG and CDC may rescue the cell death of PD-iPSC-derived mDA neurons by regulating PD-related molecular pathways. Accordingly, these results show a possibility that OASIS-mediated drug screening can be applied to a therapeutic development platform for PD.

As shown in FIGS. 11A-11F, the effects of 5 selected compounds on the light-induced α-syn aggregation in PD hiPSC-derived mDA neurons were confirmed. Opto-α-syn-mDA neurons were exposed to blue light (34 μW/mm² at 470 nm, 0.17 Hz, 0.5 s) for 7 days with or without the indicated chemical treatment. For 7 days, each drug was treated twice on day 1 and day 4. Opto-α-syn-mDA neurons were immunostained with 5G4, anti-TH, or anti-TUJ1 antibody and then subjected to quantification of (FIG. 11A) the aggregated-α-syn⁺, (FIG. 11B) TH⁺, or (FIG. 11C TUJ1⁺ area per DAPI, respectively. One-way ANOVA followed by Dunnett's post hoc test (n=12 for A, 6 images each from 2 independent experiments; n=18 for B, 6 images each from 3 independent experiments; n=12 for D, 6 images each from two independent experiments). Representative images of TH⁺ opto-α-syn-mDA neurons were shown in C. Two out of a total of 1,280 chemicals were screened by high-content imaging-mediated optogenetics-assisted method of alpha-synuclein aggregation induction system (OASIS) (FIG. 11D). Error bars represent mean±SD. Bar graph of Gene Ontology (GO) enrichment analysis. The common terms between GO terms obtained from chemogenomic analysis of BAG or CDC and from RNA-seq analysis of BAG- or CDC-responsive genes were selected. P-values of GO terms from RNA-seq analysis are displayed (FIG. 11E). Expression of selected differentially expressed genes related with GO terms in F, Heat map displays log 2 fold change values. The data was scaled by the median of each column. *P<0.05, **P<0.01, ***P<0.001, ****P<0.0001. (FIG. 11F).

The effect of 5 selected compounds on opto-α-syn-expressing PD hiPSC-derived mDA neurons was evaluated. Opto-α-syn-mDA neurons were in dark or exposed to blue light (34 μW/mm² at 470 nm, 0.17 Hz, 0.5 s) for 7 days with or without the indicated chemical treatment. For 7 days, each drug was treated twice on day 1 and day 4, and then opto-α-syn-mDA neurons were immunostained with 5G4 or anti-TUJ1 antibody.

### Example 8

### Discussion

It is widely accepted that pathogenic α-syn aggregates are important to gain an understanding of the molecular and cellular mechanisms of PD as well as the therapeutic target, but there is no sophisticated model to induce α-syn aggregation in human neurons. To address this issue, the preformed fibrils (PFFs) model has been developed based on the discovery that the injection of α-syn protofibrils to the brain could induce the formation of α-syn aggregates. However, significant expertise is needed to obtain functional PFFs. Moreover, it takes more than weeks to observe any significant PFFs-induced pathogenic α-syn aggregation even with supra-physiological quantities, and there is a lack of precise temporal control over the generation of PFFs. In this study, a synthetic biological technique to optically control the aggregation of α-syn, was developed which is called OASIS, in human neuronal cells. Importantly, OASIS generates light-induced α-syn aggregates stained with various pathological markers for α-syn-related neurodegenerative disorders, such as pS129-α-syn, Syn303, 5G4, p62, ubiquitin, and ThioS. In addition, some of pS129⁺/5G4⁺ or pS129⁺/Syn303⁺ aggregates were barely co-localized with mCherry signal; suggesting that OASIS may facilitate the formation of α-syn aggregates containing endogenous α-syn proteins. The conformation of α-syn in PD patient's brain is known to be different corresponding to different stages of maturity for Lewy pathology. In addition, recent studies have reported that various conformational antibodies of α-syn can detect different aggregates species at different stages of PD progression. The data presented herein consistently showed that 5G4 or Syn303 antibodies recognized the optically induced α-syn aggregates in early stage, and the pS129 antibody or ThioS stained α-syn aggregates in late stage, comparatively (FIG. 7A); suggesting that OASIS could initiate the α-syn aggregation processes, followed by a presession of α-syn-pathology profile related with α-syn conformational changes in short time window on the PD hiPSC-derived mDA neurons. Moreover, compared with the time to physical disease progression (perhaps decades), OASIS has a considerably shortened time for the pathological aggregate formation by using our optical induction system (hours to days). For dissecting detailed α-syn pathology, ultrastructural and functional characterization of α-syn at each stage will be needed in future studies.

In this study, two different light-responsive domains, which are Cry2 PHR and Cry2clust were tested in SH-SY5Y neuronal cells and in PD hiPSC-derived mDA neurons, respectively, to induce optical α-syn aggregations. Although Cry2 PHR-fused α-syn proteins could successfully induce pathogenic aggregates in SH-SY5Y human neuronal cells, it could not in PD hiPSC-derived mDA neurons (FIGS. 2A-2F,). Since there were several reports that robust clustering of Cry2 PHR can be weakened under certain conditions, we replaced the Cry2 PHR to Cry2clust, which is an engineered Cry2 module with C-terminal extension of 9-residue peptide from Cry2 PHR, in PD hiPSC-derived mDA neurons to induce strong homo-oligomerization. Consistent with previous study Cry2clust-fused α-syn showed rapid and efficient induction of homo-oligomerization after blue light illumination even in PD hiPSC-derived mDA neurons. Constitutively active autophagy at a basal level in neurons, which is critical for neuronal survival by degrading cargo material such as aggregate-prone proteins and damaged organelles, may partially explain the necessity of the efficient Cry2clust module in mDA neurons.

Furthermore, it was found that the α-syn aggregation in neurites was more rapidly induced compared to the cell body region (FIG. 5D). Importantly, this data recapitulated the well-known previous studies about α-syn pathology progression, which are appeared to form Lewy neurite prior to Lewy body in vivo and in vitro; suggesting that our OASIS-mediated α-syn aggregates present similar features with Lewy body and Lewy neurite. One of the advantages of OASIS is a spatial control of α-syn aggregate formation, which will be an important experimental tool to study the subcellular localization of α-syn aggregates and its relevance in PD pathogenesis. Results from the OASIS-based HCI screening of the 1,280 compounds enabled the identification of 19 compounds that reduce α-syn aggregation. Chemogenomic analyses using pathway and gene target analyses on compounds revealed common PD related characteristics among our 19 potential hit compounds. Especially, comparative studies with CRC clinical drugs and PD clinical drugs highlighted more specific PD-linked GO terms including synapse-related terms which are closely associated with α-syn protein function, ion homeostasis-related terms which are crucial for the survival of mDA neurons, and dopamine-related terms which are essential for the function of mDA neurons. Altogether, chemogenomic analyses validated the possibility of our OASIS-based HCI assays as a novel platform to find potential drugs for PD. Consequently, two small molecules were identified, CDC and BAG, as potential candidates possessing neuroprotective properties in opto-α-syn-mDA neurons. The CDC and BAG are known as a potent CCR4 chemokine receptor antagonist and PI 3-kinase/PDK1 dual inhibitor, respectively. Interestingly, several studies suggested that chemokines and chemokine receptors, including CCR4 which is expressed in microglia, astrocytes, and neurons in the central nervous system (CNS), may be involved in various neurodegenerative disorders such as Alzheimer's disease (AD), PD, multiple sclerosis (MS), stroke, and human immunodeficiency virus-associated dementia (HAD) in regards to neuroinflammation in the brain. Consistently, PD patients showed an elevated level of CCL5, one of the CCR4 ligands, compared to the controls. In addition, it has been reported that activation of metabotropic glutamate/P3K/AKT signaling pathway may play an important role in the pathogenesis of PD. Collectively, this study suggests a possibility that CCR4 and PI3K pathways could be novel targets for drug development in PD.

When analyzing RNA-seq results from CDC- or BAG-treated samples, common GO terms and gene lists belonging to these terms were identified (FIGS. 11E and 11F). BAG and CDC treatment reversed the expression levels of a set of genes that were changed by light-induced α-syn aggregates, to their expression levels of the dark condition. Importantly, these genes are categorized in the PD-related GO terms such as regulation of ion transport or purine nucleotide binding. Interestingly, the GO term analysis result from RNA-seq analysis was consistent with the result from the target prediction of CDC and BAG through SEA tools (Tables 2-4). However, it is still unclear how specifically these compounds would block the formation of α-syn aggregates and/or accelerate the degradation of already formed aggregates; detailed mechanisms need to be elucidated for the future study. OASIS has several advantages that can reinvigorate current PD research. Firstly, this study demonstrates that OASIS-based HCI assay can be used as a novel screening platform to identify small molecules that can reduce the levels of α-syn aggregation and reverse the cytotoxicity in PD hiPSC-derived mDA neurons. By calculating AIS, 19 molecules out of 1,280 compounds were successfully screened. Notably, 4 of 19 compounds have been already published as a potential therapeutic drug for PD, confirming that OASIS can be utilized as an efficient method for discovering new targets for PD in a high throughput manner. Secondly, it can provide a unique window to identify genetic targets that control 3-sheet structure formation of α-syn by combining OASIS with genome-scale knockout and transcriptional activation screening. After infecting opto-α-syn expressing neuronal cells with CRISPR-based lentiviral libraries, infected cells which show significantly down- or up-regulated level of ThioS⁺ or 5G4⁺ aggregates with blue light illumination can be isolated and analyzed by high-throughput sequencing of barcodes to quantify each sgRNAs. Thirdly, by genetically applying OASIS into the mouse model through CRISPR/Cas9-mediated homologous recombination, precise spatiotemporal control of α-synaggregation may be possible in vivo. Lastly, although our current study focuses only on the α-syn aggregation in PD, most neurodegenerative diseases are patho-physiologically associated with protein aggregation: A3 and tau in AD, α-syn in PD and dementia with LBs, huntingtin in Huntington's disease, ataxins in polyglutamine diseases, prions in prion diseases, SOD1 and TDP43 in amyotrophic lateral sclerosis and frontotemporal lobar degeneration (FLD), and tau in frontotemporal dementia and FLD. The processes of protein aggregation in each neurodegenerative disease are exceedingly complex and occur over a considerable amount of time, and thus, revealing both the mechanisms of formation and the pathophysiological effects of protein aggregation are challenging due to a lack of proper model systems. Accordingly, the opto-aggregation system described herein can be applied to various other diseases with pathogenic protein aggregations.

In summary, the OASIS provides a highly efficient and rapid humanized neuronal model to study pathophysiological α-syn aggregation using optical stimulation. Furthermore, newly developed OASIS-based HCI assay can be expendably applied for screening of novel compounds curing synucleinopathy-related diseases with various cell types differentiated from hiPSCs.

### Example 9

Validation of Selected Compounds Bag 956 and CDC 021 Efficacies in A-Syn PFF Models on Mouse Primary Neurons and Human PD IPSC-Derived MDA Neurons

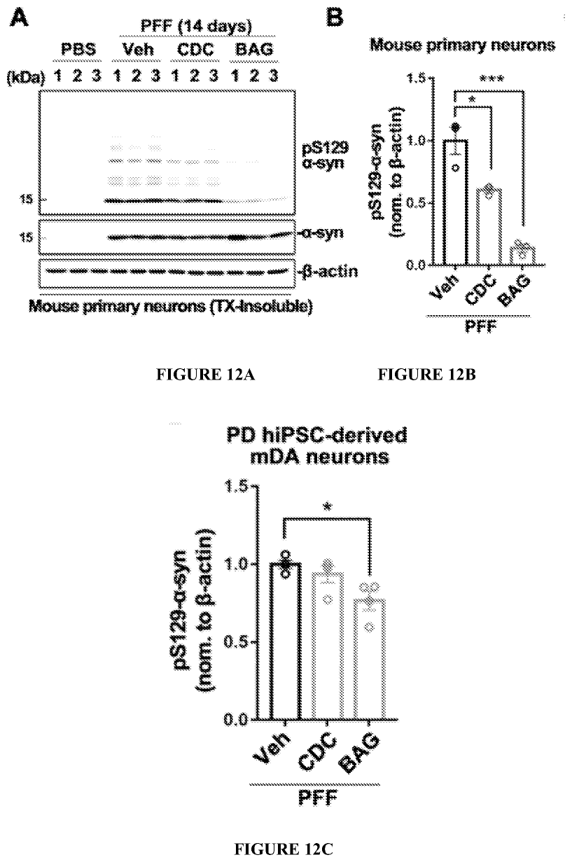

To validate the effects of these two compounds on the OASIS-independent model, α-syn PFF were treated with mouse primary neurons and non-OASIS-human PD hiPSC-derived mDA neurons with CDC 021 and BAG 956. Treatment with CDC 021 and BAG 956 significantly reduced the pathologic α-syn in the Triton X-100 (TX)-insoluble fraction (FIGS. 12A and 12B); these results were confirmed by immunocytochemistry in mouse primary neurons in which representative immunostaining images of MAP2 and pS129-α-syn in mouse primary neurons treated with PBS or α-syn PFF with vehicle, BAG 956, or CDC 021, were analyzed (data not known). It was also confirmed that BAG 956 treatment showed a significant decrease of TX-insoluble pS129-α-syn in human PD iPSC-derived mDA neurons (FIG. 12C).

### Example 10

### Validation of Neuroprotective Effect of Bag on A-Syn PFF-Induced Behavioral Deficits

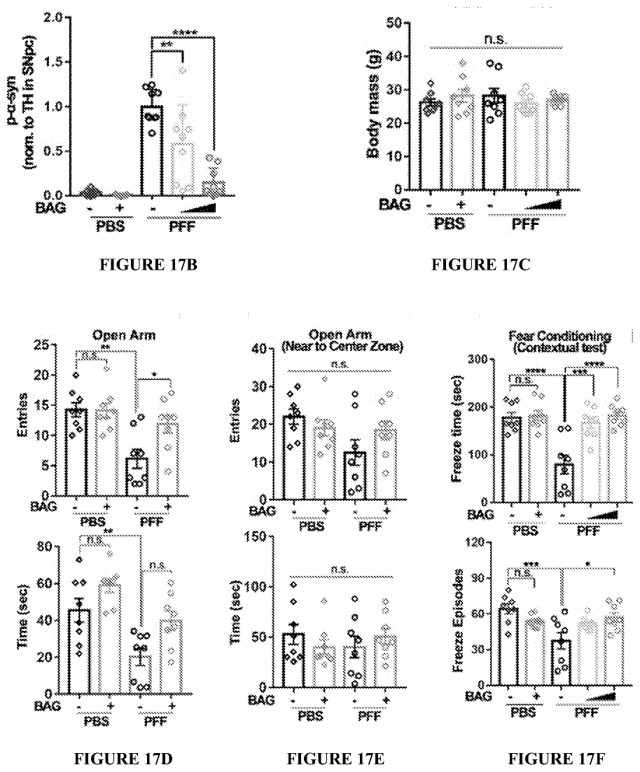

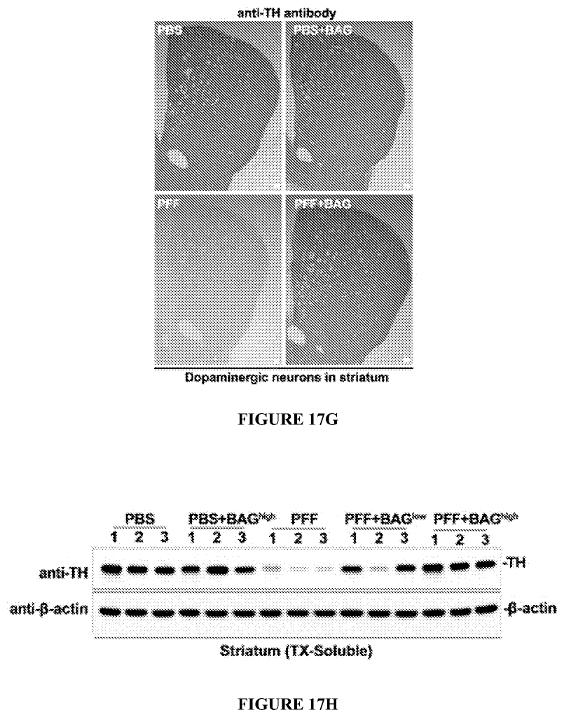

To test the neuroprotective potential of BAG 956, in vivo α-syn PFF mouse model was used for a short-term period. Immunostaining analysis with PBS or BAG 956 treatment for two months at 2 mg/kg or one month at 10 mg/kg after the intrastriatal α-syn PFF injection showed that BAG 956 significantly reduced pathologic pS129-α-syn immunoreactivity induced by α-syn PFF injection in TW neurons of the ventral midbrain (FIGS. 17A-17B). Next, BAG 956 efficacy was validated for long-term treatment on α-syn PFF mouse model by comprehensive behavioral and biochemical assays.

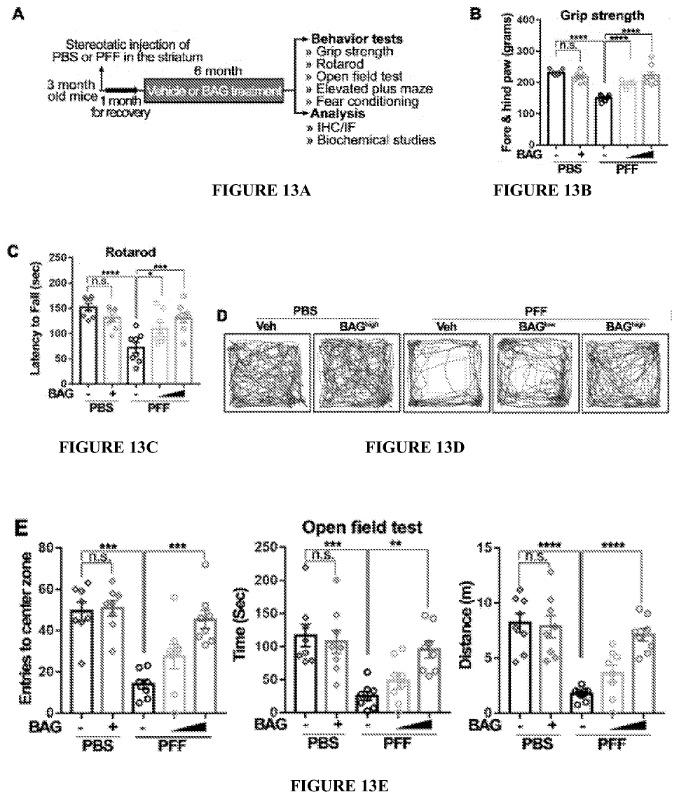

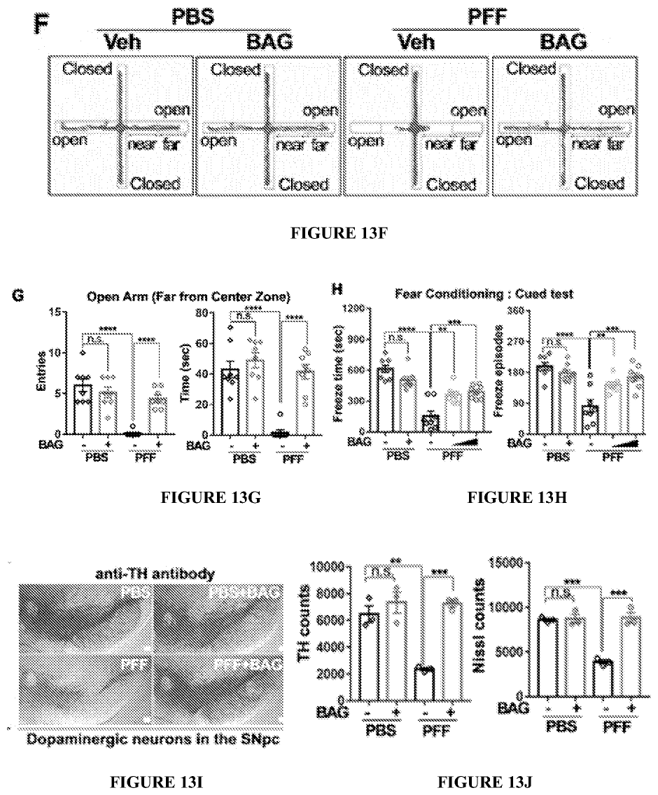

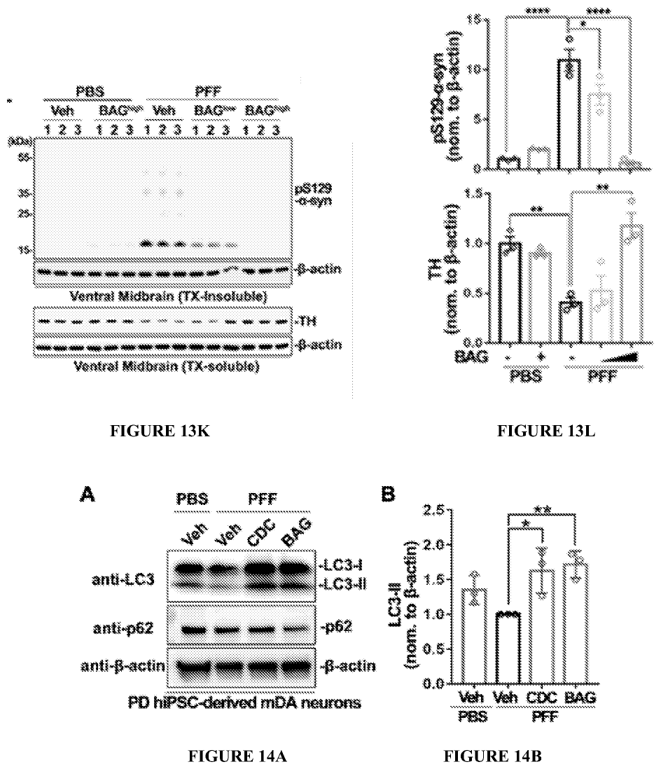

Behavioral analysis was performed to evaluate the behavioral defect elicited by intrastriatal α-syn PFF injection and any restoration of behavioral dysfunction by oral administration of BAG 956 for 6 months at a low (2 mg/kg) or high (10 mg/kg) concentration after one-month recovery from α-syn PFF injection. PBS injected mice were treated vehicle or BAG956^{high} (10 mg/kg), whereas α-syn PFF injected mice were orally administered with vehicle, BAG 956^{low} (2 mg/kg), or BAG956^{high} (10 mg/kg) (FIG. 13A). No endpoint body mass differences were observed in all groups (FIG. 17C). Intrastriatal α-syn PFF injected model showed significant reduction of fore- and hind-limb grip strength (FIG. 13B), latency to fall from the rotarod test (FIG. 13C—the values in α-syn PFF with vehicle-treated group was set as 1.0); these were dramatically recovered with treatment of BAG 956 at both 2 mg/kg and 10 mg/kg doses. The open field test (OFT) and elevated plus maze (EPM) were performed to evaluate locomotion, exploration, and anxiety behaviors. Representative images showed that the α-syn PFF injected mice preferred to stay closer to the wall of the designated area (FIGS. 13D and 13E) or the closed arm of the EPM (FIGS. 13F and 13G). The time spent, number of entries, and travelled distance in the center zone of the OFT (FIGS. 13E and 17D) or in the far zone of the open arm of the EPM (FIGS. 13G and 17E), were significantly reduced in the PFF mouse model. The behavioral abnormalities were significantly restored with treatment of BAG 956 at 10 mg/kg. Next, hippocampal- or amygdala-dependent learning and memory based contextual or cued fear conditioning tests were performed. The α-syn PFF injected mouse model showed dampened freezing phenotypes including total freezing time and freezing episode (FIGS. 13H, 13L and 17F); these were recovered by BAG 956 treatment at both 2 mg/kg and 10 mg/kg doses.

### Example 11

### Validation of Neuroprotective Efficacy of Bag 956 on A-Syn PFF-Induced PD-Like Pathologies In Vivo

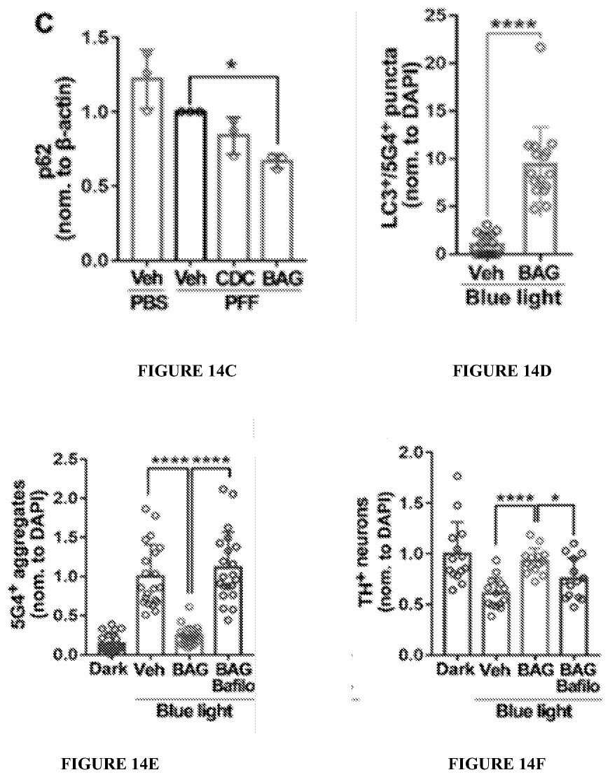

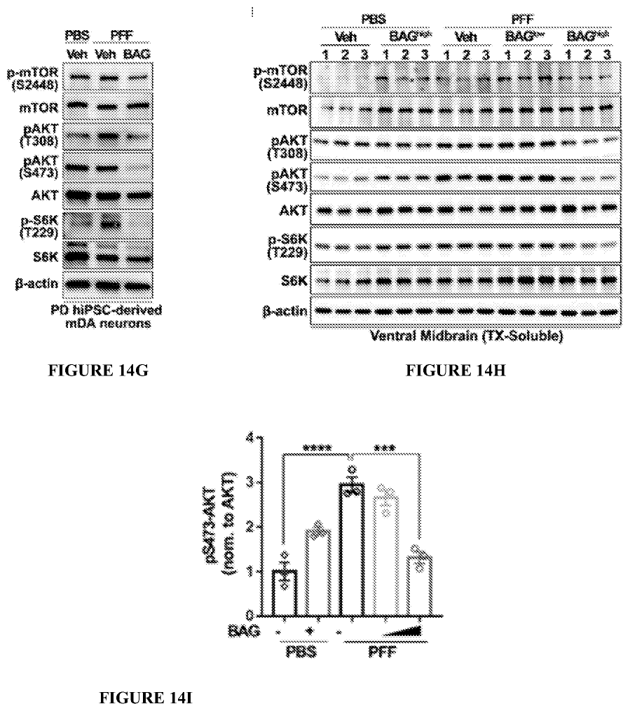

To examine whether the oral administration of BAG 956 could rescue the loss of DA neurons induced by intrastriatal α-syn PFF injection, the number of TH-positive neurons in the substantia nigra pars compacta (SNpc) was measured via an unbiased stereological counting analysis. Representative TH immune-stained images of the SNr (FIG. 13I) and quantification of the number of TH- and Nissl-positive stained DA neurons (FIG. 13J) revealed a significant loss of DA neurons in α-syn PFF injected mice; this was recovered by treatment with BAG 956 at 10 mg/kg. Importantly, α-syn PFF injection significantly reduced striatal TH-positive fiber density as assessed by IHC, which was rescued by treatment with BAG 956 at 10 mg/kg (FIG. 14G). Pathologic pSer129-α-syn immunoreactivity was elevated in TH-positive neurons of the SN of α-syn PFF injected mice as assessed by IHC; this was significantly rescued by treatment with BAG 956 at 10 mg/kg (FIG. 17G). Similar results were observed in WB analysis with TX-insoluble fractions from the ventral midbrains, α-syn PFF injection increased the levels of pS129-α-syn in the TX-insoluble fraction, which was significantly reduced by BAG 956 treatment at both 2 mg/kg and 10 mg/kg doses (FIGS. 13K and 13L, upper panels). α-syn PFF injection reduced the protein levels of TH in both the ventral midbrain and striatum, which were rescued by BAG 956 treatment at 10 mg/kg (FIGS. 13K and 13L, lower panels, and FIG. 171H).

### Example 12

### Autophagy-Enhancing Potential of Bag 956 on Neuroprotective Effects Against A-Syn Pathology

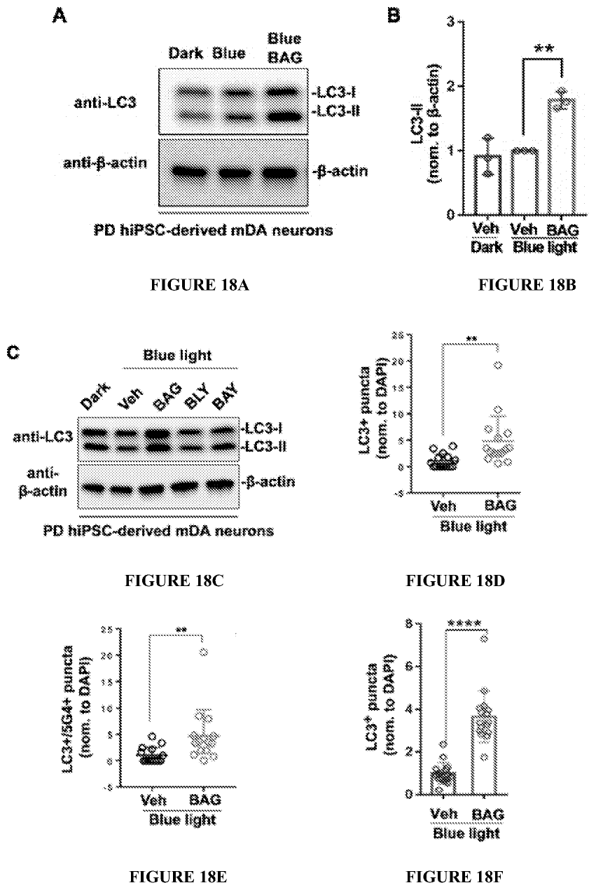

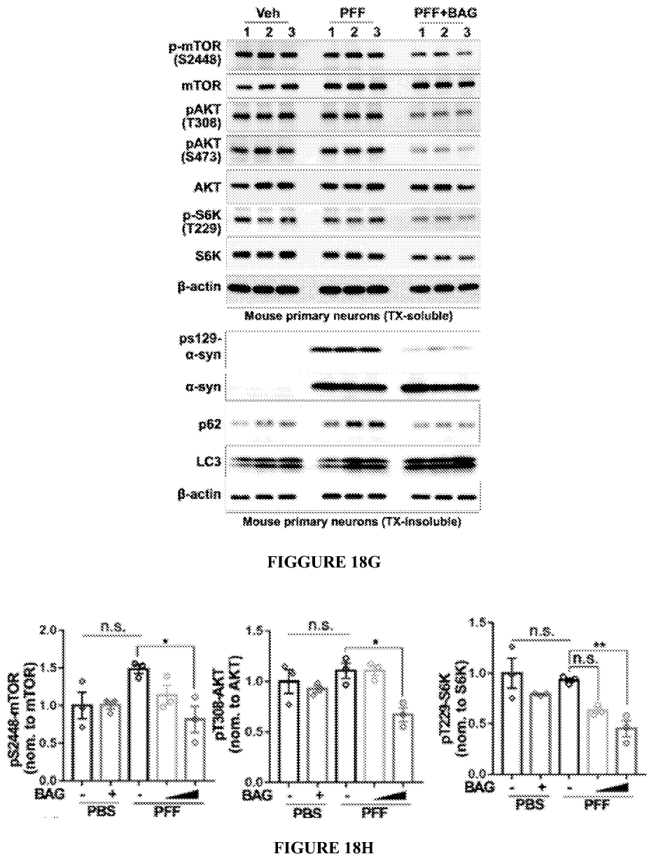

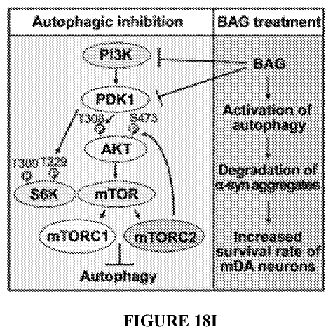

Previous studies demonstrated that α-syn was degraded by autophagy, and autophagic defects exacerbate α-syn-mediated PD pathology. Because autophagy can be activated by the inhibition of PI 3-kinase (PI3K) or the PDK1 pathway, it was tested whether treatment with BAG 956 can induce the autophagic flux to decrease α-syn aggregates in opto-α-syn mDA neurons and MOs. Interestingly, only BAG 956 treatment up-regulated and down-regulated the level of autophagic marker LC-311 and autophagy flux protein p62, respectively, compared with control or CDC 021 treatment in α-syn PFF-treated opto-α-syn-mDA neurons (FIGS. 14A-14C). The level of LC-311 was also increased by BAG 956 treatment in blue light-illuminated opto-α-syn-mDA neurons (FIGS. 18A and 18B), and it was more potent than BYL719 (BLY, alpelisib) or BAY 80-6946 (BAY, copanlisib); both of these are pan-PI3K inhibitors approved by FDA (FIG. 18C). It was further confirmed that BAG 956 treatment treated mDA neurons showed the increased number of LC3′ puncta as well as LC3⁺/5G4⁺ co-localized vesicles (FIGS. 18D-18F). These results were consistent when BAG 956 was only treatment during the dark condition after blue light illumination for six days in opto-α-syn-MOs (FIGS. 14D and 14F), suggesting that autophagy is involved in BAG 956-mediated degradation of opto-α-syn aggregates. Consistently, additional treatment with bafilomycin A1, the autophagy inhibitor, with BAG 956 reversed the reduction of 5G4′ α-syn aggregates as well as the level of TH⁺ mDA neurons in blue light-illuminating condition (FIGS. 14E and 14F), suggesting autophagic degradation is important for the BAG 956-mediated neuroprotective effects against α-syn pathology.

Since PI3K-PDK1/AKT/mTOR signaling pathway plays a critical role in regulation of autophagy, it was investigated the effects of BAG 956 treatments on PFF models. Phosphorylation of many PDK1 downstream targets, PI3K, AKT, S6K, and mTOR, were significantly reduced by BAG 956 treatment in α-syn PFF-treated PD hiPSC-derived dopaminergic neurons (FIG. 14G) and mouse primary neurons (FIG. 18G) as well as α-syn PFF-injected mice (FIGS. 14H and 18H). Importantly, α-syn PFF injection significantly increased pS473-AKT, which was reduced by BAG 956 treatment in α-syn PFF injected mice (FIG. 14I). Taken together, BAG 956 may act as an autophagy-enhancing drug through inhibition of the PI3K-PDK1/AKT/mTOR signaling pathway (FIG. 18I).

### Example 13

### Rescuing Effect of Bag 956 in Tau PFF-Treated Neuron Model In Vitro and In Vivo

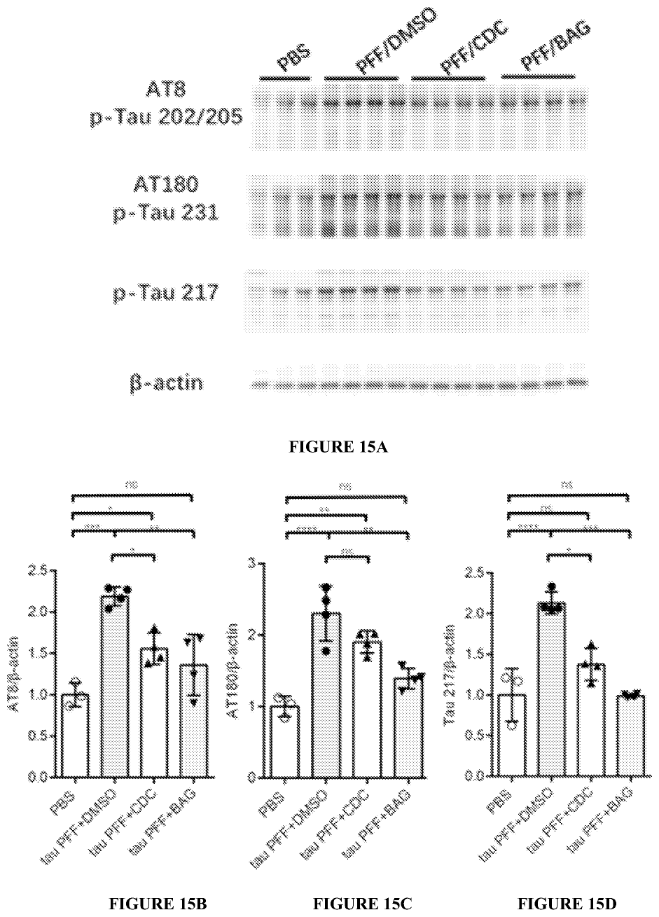

It was tested whether the BAG956 compound can be beneficial in other proteinopathy models. The BAG and CDC compounds were used on the tau PFF treated mouse conical neurons (FIGS. 15A-15D) and measured expression levels with three different phosphorylated-Tau (p-Tau) antibodies. Interestingly, all three forms of p-Tau were significantly downregulated by BAG treatment. These data suggested that the beneficial effects of BAG could also be applied to tau aggregates in vitro.

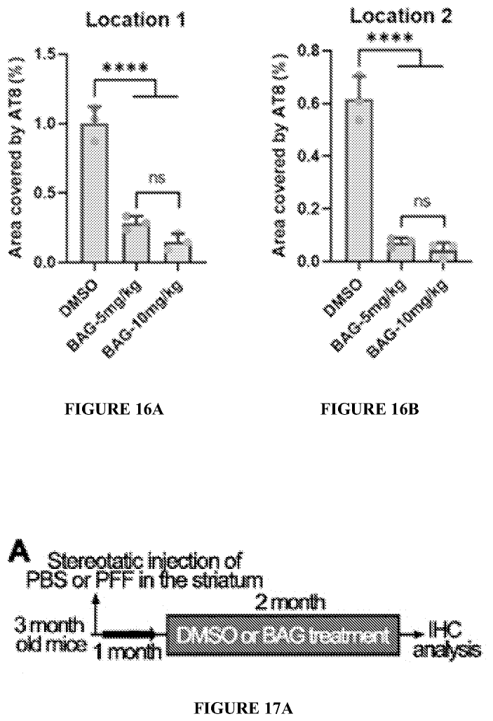

To find in vivo efficacy in a pilot exponential set and set a drug dosage, an in vivo experiment with the tau-PFF injected mice with DMSO or BAG treatment (5 and 10 μg/kg) was performed. Intraperitoneal injection of different concentrations (0, 5, 10 mg/kg) of BAG was given for one month, once every two days. Immunofluorescence staining results of AT8 in the dentate gyrus of C57bl/6j mice (3 per group) after injection of K18-tau PFF (5 μg/kg) into the hippocampus for two months was performed (data not shown). As shown in FIGS. 16A and 16B, significantly decreased levels of the AT8+ phosphorylated-tau were observed in the tau-PFF injected mice with BAG treatment. These data present the therapeutic effects of the BAG efficacy test in the tau-PFF injected mice without noticeable toxicity with 10 μg/kg dosage.

### Example 14

### Discussion

An optically controllable α-syn aggregation inducing system (OASIS) was initially developed in human neuronal cells. Importantly, OASIS generated light-induced α-syn aggregates stained with various pathological markers for PD. Moreover, OASIS has a considerably shortened time for the pathological aggregate formation (hours to days), which enabled us to develop an OASIS-based HCI screening assay. The OASIS-based screening of the 1,280 compounds identified two potential neuroprotective molecules, CDC 021, and BAG 956. BAG 956, in particular, successfully rescued the α-syn pathology induced by α-syn PFF (an OASIS-independent PD model), which has been extensively used to study α-syn pathology and PD in vitro and in vivo. Importantly, the efficacy of BAG 956 on α-syn PFF-induced PD-like symptom was validated in in vivo mouse model by a wide range of pathological, behavioral, and biochemical studies suggesting BAG 956 could be a potent candidate for alleviating PD symptoms. Furthermore, it was found that BAG 956 activated autophagic flux by inhibiting the PI3K-PDK/AKT/mTOR signaling pathway to induce clearance of α-syn aggregates. In particular, pS473-AKT, which is the target of mTOR complex 2 (mTORC2), was the most effective target by BAG 956 treatment, suggesting BAG 956 may enhance autophagy by regulation of mTORC2-dependent mechanism without side effects induced by inhibition of mTORC1.

In addition, to address if BAG 956 can be effective other pathogenic protein aggregates, BAG 956 was tested in a tau PFF model as tau aggregates can be found in many different forms of dementia. As shown in FIGS. 15A-15D and 16A-16B, BAG 956 treatment can significantly decrease the levels of pathogenic form of tau PFF in vitro and in vivo.

In summary, OASIS identified a novel compound, BAG 956, rescuing α-synucleinopathy-related phenotypes of in vitro and in vivo PD models, as well as tau PFF pathology.

### REFERENCES

Akoglu, H. (2018). User's guide to correlation coefficients. Turk. J. Emerg. Med. 18, 91-93.
Anderson, J. P., Walker, D. E., Goldstein, J. M., de Laat, R., Banducci, K., Caccavello, R. J., Barbour, R., Huang, J., Kling, K., Lee, M., et al. (2006). Phosphorylation of Ser-129 is the dominant pathological modification of alpha-synuclein in familial and sporadic Lewy body disease. J. Biol. Chem. 281, 29739-29752.
Badger, J. L., Cordero-Llana, O., Hartfield, E. M., and Wade-Martins, R. (2014). Parkinson's disease in a dish—Using stem cells as a molecular tool. Neuropharmacology 76 Pt A, 88-96. Barnham, K. J., and Bush, A. I. (2008). Metals in Alzheimer's and Parkinson's diseases. Curr. Opin. Chem. Biol. 12, 222-228.
Blesa, J., and Przedborski, S. (2014). Parkinson's disease: animal models and dopaminergic cell vulnerability. Front. Neuroanat. 8, 155.
Braak, H., Del Tredici, K., Rub, U., de Vos, R. A., Jansen Steur, E. N., and Braak, E. (2003). Staging of brain pathology related to sporadic Parkinson's disease. Neurobiol. Aging 24, 197-211.
Burre, J. (2015). The synaptic function of alpha-synuclein. J. Parkinsons Dis. 5, 699-713.
Che, D. L., Duan, L., Zhang, K., and Cui, B. (2015). The dual characteristics of light-induced cryptochrome 2, homo-oligomerization and heterodimerization, for optogenetic manipulation in mammalian cells. ACS Synth. Biol. 4, 1124-1135.
Chen, L., and Feany, M. B. (2005). Alpha-synuclein phosphorylation controls neurotoxicity and inclusion formation in a Drosophila model of Parkinson disease. Nat. Neurosci. 8,657-663.
Chu, Y., Muller, S., Tavares, A., Barret, O., Alagille, D., Seibyl, J., Tamagnan, G., Marek, K., Luk, K. C., Trojanowski, J. Q., et al. (2019). Intrastriatal alpha-synuclein fibrils in monkeys: spreading, imaging and neuropathological changes. Brain 142, 3565-3579.
Cornacchia, D., and Studer, L. (2017). Back and forth in time: Directing age in iPSC-derived lineages. Brain Res. 1656, 14-26.
Covell, D. J., Robinson, J. L, Akhtar, R. S., Grossman, M., Weintraub, D., Bucklin, H. M., Pitkin, R. M., Riddle, D., Yousef, A., Trojanowski, J. Q., et al. (2017). Novel conformation-selective alpha-synuclein antibodies raised against different in vitro fibril forms show distinct patterns of Lewy pathology in Parkinson's disease. Neuropathol. Appl. Neurobiol. 43,604-620.
Dauer, W., and Przedborski, S. (2003). Parkinson's disease: mechanisms and models. Neuron 39, 889-909.
Delenclos, M., Burgess, J. D., Lamprokostopoulou, A., Outeiro, T. F., Vekrellis, K., and McLean, P. J. (2019). Cellular models of alpha-synuclein toxicity and aggregation. J. Neurochem. 150,566-576.
Devine, M. J., Ryten, M., Vodicka, P., Thomson, A. J., Burdon, T., Houlden, H., Cavaleri, F., Nagano, M., Drummond, N.J., Taanman, J. W., et al. (2011). Parkinson's disease induced pluripotent stem cells with triplication of the alpha-synuclein locus. Nat. Commun. 2, 440.
Dickerson, J. W., and Conn, P. J. (2012). Therapeutic potential of targeting metabotropic glutamate receptors for Parkinson's disease. Neurodegener. Dis. Manag. 2, 221-232.
Duda, J., Potschke, C., and Liss, B. (2016). Converging roles of ion channels, calcium, metabolic stress, and activity pattern of Substantia nigra dopaminergic neurons in health and Parkinson's disease. J. Neurochem. 139 Suppl 1, 156-178.
Dzamko, N., Gysbers, A., Perera, G., Bahar, A., Shankar, A., Gao, J., Fu, Y., and Halliday, G. M. (2017). Toll-like receptor 2 is increased in neurons in Parkinson's disease brain and may contribute to alpha-synuclein pathology. Acta Neuropathol. 133, 303-319.
Fedele, S., Collo, G., Behr, K., Bischofberger, J., Muller, S., Kunath, T., Christensen, K., Gundner, A. L., Graf, M., Jagasia, R., et al. (2017). Expansion of human midbrain floor plate progenitors from induced pluripotent stem cells increases dopaminergic neuron differentiation potential. Sci. Rep. 7, 6036.
Giasson, B. I., Duda, J. E., Quinn, S. M., Zhang, B., Trojanowski, J. Q., and Lee, V. M. (2002). Neuronal alpha-synucleinopathy with severe movement disorder in mice expressing A53T human alpha-synuclein. Neuron 34, 521-533.
Gorell, J. M., Johnson, C. C., Rybicki, B. A., Peterson, E. L., Kortsha, G. X., Brown, G. G., and Richardson, R. J. (1997). Occupational exposures to metals as risk factors for Parkinson's disease. Neurology 48, 650-658.
Guo, J. L., Covell, D. J., Daniels, J. P., Iba, M., Stieber, A., Zhang, B., Riddle, D. M., Kwong, L. K., Xu, Y., Trojanowski, J. Q., et al. (2013). Distinct alpha-synuclein strains differentially promote tau inclusions in neurons. Cell 154, 103-117.
Hartmann, A. (2004). Postmortem studies in Parkinson's disease. Dialogues Clin. Neurosci. 6, 281-293.
Hedya, S. A., Safar, M. M., and Bahgat, A. K. (2018). Cilostazol mediated Nurrl and autophagy enhancement: neuroprotective activity in rat rotenone PD model. Mol. Neurobiol. 55, 7579-7587.
Hockemeyer, D., Soldner, F., Beard, C., Gao, Q., Mitalipova, M., DeKelver, R. C., Katibah. G. E., Amora, R., Boydston, E. A., Zeitler, B., el al. (2009). Efficient targeting of expressed and silent genes in human ESCs and iPSCs using zinc-finger nucleases. Nat. Biotechnol. 27, 851-857.
Jones, D. R., Delenclos, M., Baine, A. T., DeTure, M., Murray, M. E., Dickson, D. W., and McLean, P. J. (2015). Transmission of soluble and insoluble alpha-synuclein to mice. J. Neuropathol. Exp. Neurol. 74, 1158-1169.
Kalia, S. K., Kalia, L. V., and McLean, P. J. (2010). Molecular chaperones as rational drug targets for Parkinson's disease therapeutics. CNS Neurol. Disord. Drug Targets 9, 741-753.
Kanazawa, T., Uchihara, T., Takahashi, A., Nakamura, A., Orimo, S., and Mizusawa, H. (2008). Three-layered structure shared between Lewy bodies and lewy neurites-three-dimensional reconstruction of triple-labeled sections. Brain Pathol. 18, 415-422.
Kennedy, M. J., Hughes, R. M., Peteya, L. A., Schwartz, J. W., Ehlers, M. D., and Tucker, C. L. (2010). Rapid blue-light-mediated induction of protein interactions in living cells. Nat. Methods 7, 973-975.
Kim, N., Kim, J. M., Lee, M., Kim, C. Y., Chang, K. Y., and Heo, W. D. (2014). Spatiotemporal control of fibroblast growth factor receptor signals by blue light. Chem. Biol. 21, 903-912.
Koprich, J. B., Kalia, L. V., and Brotchie, J. M. (2017). Animal models of alpha-synucleinopathy for Parkinson disease drug development. Nat. Rev. Neurosci./8, 515-529.
Kouroupi, G., Taoufik, E., Vlachos, I. S., Tsioras, K., Antoniou, N., Papastefanaki, F., Chroni-Tzartou, D., Wrasidlo, W., Bohl, D., Stellas, D., et al. (2017). Defective synaptic connectivity and axonal neuropathology in a human iPSC-based model of familial Parkinson's disease. Proc. Natl. Acad. Sci. U.S.A 114, E3679-E3688.
Kovacs, G. G., Wagner, U., Dumont, B., Pikkarainen, M., Osman, A. A., Streichenberger, N., Leisser, I., Verchere, J., Baron, T., Alafuzoff, I., et al. (2012). An antibody with high reactivity for disease-associated alpha-synuclein reveals extensive brain pathology. Acta Neuropathol. 124, 37-50.
Kriks, S., Shim, J. W., Piao, J., Ganat, Y. M., Wakeman, D. R., Xie, Z., Carrillo-Reid, L., Auyeung, G., Antonacci, C., Buch, A., et al. (2011). Dopamine neurons derived from human ES cells efficiently engraft in animal models of Parkinson's disease. Nature 480, 547-551.
Lee, S., Park, H., Kyung, T., Kim, N.Y., Kim, S., Kim, J., and Heo, W. D. (2014). Reversible protein inactivation by optogenetic trapping in cells. Nat. Methods 11, 633-636.
Lee, V. M., and Trojanowski, J. Q. (2006). Mechanisms of Parkinson's disease linked topathological alpha-synuclein: new targets for drug discovery. Neuron 52, 33-38.
Liu, G. H., Ding, Z., and lzpisua Belmonte, J. C. (2012). iPSC technology to study human aging and aging-related disorders. Curr. Opin. Cell Biol. 24, 765-774.
Luk, K. C., Kehm, V., Carroll, J., Zhang, B., O'Brien, P., Trojanowski, J. Q., and Lee, V. M. (2012). Pathological alpha-synuclein transmission initiates Parkinson-like neurodegeneration in nontransgenic mice. Science 338, 949-953.
Luk, K. C., Song, C., O'Brien, P., Stieber, A., Branch, J. R., Brunden, K. R., Trojanowski, J. Q., and Lee, V. M. (2009). Exogenous alpha-synuclein fibrils seed the formation of Lewy body-like intracellular inclusions in cultured cells. Proc. Nati. Acad. Sci. U.S.A 106, 20051-20056.
Majbour, N. K., Vaikath, N. N., van Dijk, K. D., Ardah, M. T., Varghese, S., Vesterager, L. B., Montezinho, L. P., Poole, S., Safieh-Garabedian, B., Tokuda, T., et al. (2016). Oligomeric and phosphorylated alpha-synuclein as potential CSF biomarkers for Parkinson's disease. Mol. Neurodegener. 11, 7.
Mali, P., Yang, L., Esvelt, K. M., Aach, J., Guell, M., DiCarlo, J. E., Norville, J. E., and Church, G. M. (2013). RNA-guided human genome engineering via Cas9. Science 339, 823-826.
Matsuda, S., Nakanishi, A., Wada, Y., and Kitagishi, Y. (2013). Roles of PI3K/AKT/PTEN pathway as a target for pharmaceutical therapy. Open Med. Chem. J. 7, 23-29.
McGeer, P. L., Itagaki, S., Boyes, B. E., and McGeer, E. G. (1988). Reactive microglia are positive for HLA-DR in the substantia nigra of Parkinson's and Alzheimer's disease brains. Neurology 38, 1285-1291.
McLeary, F. A., Rcom-H'cheo-Gauthier, A. N., Kinder, J., Goulding, M., Khoo, T. K., Mellick, G. D., Chung, R. S., and Pountney, D. L. (2018). Dexamethasone inhibits copper-induced alpha-synuclein aggregation by a metallothionein-dependent mechanism. Neurotox. Res. 33, 229-238.
Miller, J. D., Ganat, Y. M., Kishinevsky, S., Bowman, R. L., Liu, B., Tu, E. Y., Mandal, P. K., Vera, E., Shim, J. W., Kriks, S., et al. (2013). Human iPSC-based modeling of late-onset disease via progerin-induced aging. Cell Stem Cell 13, 691-705.
Mines, M., Ding, Y., and Fan, G. H. (2007). The many roles of chemokine receptors in neurodegenerative disorders: emerging new therapeutical strategies. Curr. Med. Chem. 14, 2456-2470.
Mogi, M., Harada, M., Kondo, T., Riederer, P., and Nagatsu, T. (1996). Interleukin-2 but not basic fibroblast growth factor is elevated in parkinsonian brain. Short communication. J. Neural Transm. (Vienna) 103, 1077-1081.
Navarro, G., Borroto-Escuela, D. O., Fuxe, K., and Franco, R. (2016). Purinergic signaling in Parkinson's disease. Relevance for treatment. Neuropharmacology 104, 161-168.
Oh, Y. (2019). Patient-specific pluripotent stem cell-based Parkinson's disease models showing endogenous alpha-synuclein aggregation. BMB Rep. 52, 349-359.
Oh, Y., Cho, G. S., Li, Z., Hong, I., Zhu, R., Kim, M. J., Kim, Y. J., Tampakakis, E., Tung, L., Huganir, R., et al. (2016). Functional coupling with cardiac muscle promotes maturation of hPSC-derived sympathetic neurons. Cell Stem Cell 19, 95-106.
Olanow, C. W., and Tatton, W. G. (1999). Etiology and pathogenesis of Parkinson's disease. Annu. Rev. Neurosci. 22, 123-144.
Orimo, S., Uchihara, T., Nakamura, A., Mori, F., Kakita, A., Wakabayashi, K., and Takahashi, H. (2008). Axonal alpha-synuclein aggregates herald centripetal degeneration of cardiac sympathetic nerve in Parkinson's disease. Brain 131, 642-650.
Oueslati, A. (2016). Implication of alpha-synuclein phosphorylation at SI29 in synucleinopathies: what have we learned in the last decade?J. Parkinsons Dis. 6, 39-51.
Park, H., Kim, N.Y., Lee, S., Kim, N., Kim, J., and Heo, W. D. (2017). Optogenetic protein clustering through fluorescent protein tagging and extension of CRY2. Nat. Commun. 8, 30.
Peng, C., Gathagan, R. J., Covell, D. J., Medellin, C., Stieber, A., Robinson, J. L., Zhang, B., Pitkin, R. M., Olufemi, M. F., Luk, K. C., et al. (2018). Cellular milieu imparts distinct pathological alpha-synuclein strains in alpha-synucleinopathies. Nature 557, 558-563.
Polymeropoulos, M. H., Lavedan, C., Leroy, E., Ide, S. E., Dehejia, A., Dutra, A., Pike, B., Root, H., Rubenstein, J., Boyer, R., et al. (1997). Mutation in the alpha-synuclein gene identified in families with Parkinson's disease. Science 276, 2045-2047.
Qin, H., Buckley, J. A., Li, X., Liu, Y., Fox, T. H., 3rd, Meares, G. P., Yu, H., Yan, Z., Harms, A. S., Li, Y., et al. (2016). Inhibition of the JAK/STAT pathway protects against alpha-synuclein-induced neuroinflammation and dopaminergic neurodegeneration. J. Neurosci. 36, 5144-5159.
Rost, B. R., Schneider-Warme, F., Schmitz, D., and Hegemann, P. (2017). Optogenetic tools for subcellular applications in neuroscience. Neuron 96, 572-603.
Shipley, M. M., Mangold, C. A., and Szpara, M. L. (2016). Differentiation of the SH-SY5Y human neuroblastoma cell line. J. Vis. Exp., 53193.
Singleton, A. B., Farrer, M., Johnson, J., Singleton, A., Hague, S., Kachergus, J., Hulihan, M., Peuralinna, T., Dutra, A., Nussbaum, R., et al. (2003). alpha-Synuclein locus triplication causes Parkinson's disease. Science 302, 841.
Tang, P., Chong, L., Li, X., Liu, Y., Liu, P., Hou, C., and Li, R. (2014). Correlation between serum RANTES levels and the severity of Parkinson's disease. Oxid. Med. Cell. Longev. 2014, 208408.
Thul, P. J., Akesson, L., Wiking, M., Mahdessian, D., Geladaki, A., Ait Blal, H., Alm, T., Asplund, A., Bjork, L., Breckels, L. M., et al. (2017). A subcellular map of the human proteome. Science 356.
Tran, H. T., Chung, C. H., lba, M., Zhang, B., Trojanowski, J. Q., Luk. K. C., and Lee, V. M. (2014). Alpha-synuclein immunotherapy blocks uptake and templated propagation of misfolded alpha-synuclein and neurodegeneration. Cell Rep. 7, 2054-2065.
Volpicelli-Daley, L. A., Luk, K. C., Patel, T. P., Tanik, S. A., Riddle, D. M., Stieber, A., Meaney, D. F., Trojanowski, J. Q., and Lee, V. M. (2011). Exogenous alpha-synuclein fibrils induce Lewy body pathology leading to synaptic dysfunction and neuron death. Neuron 72, 57-71.
Waxman, E. A., Duda, J. E., and Giasson, B.1. (2008). Characterization of antibodies that selectively detect alpha-synuclein in pathological inclusions. Acta Neuropathol. 116, 37-46.
West, A. B., Moore, D. J., Choi, C., Andrabi, S. A., Li, X., Dikeman, D., Biskup, S., Zhang, Z., Lim, K. L., Dawson, V. L., et al. (2007). Parkinson's disease-associated mutations in LRRK2 link enhanced GTP-binding and kinase activities to neuronal toxicity. Hum. Mol. Genet. 16, 223-232.
Woodard, C. M., Campos, B. A., Kuo, S. H., Nirenberg, M. J., Nestor, M. W., Zimmer, M., Mosharov, E. V., Sulzer, D., Zhou, H., Paull, D., et al. (2014). iPSC-derived dopamine neurons reveal differences between monozygotic twins discordant for Parkinson's disease. Cell Rep. 9, 1173-1182.
W. Dauer, S. Przedborski, Parkinson's disease: mechanisms and models. Neuron 39, 889-909 (2003).
J. D. Miller et al., Human iPSC-based modeling of late-onset disease via progerin-induced aging. Cell Stem Cell 13, 691-705 (2013).
K. C. Luk et al., Exogenous alpha-synuclein fibrils seed the formation of Lewy body-like intracellular inclusions in cultured cells. Proc. Natl. Acad. Sci. U.S.A 106, 20051-20056 (2009).
B. A. Seo et al., TRIP12 ubiquitination ofglucocerebrosidase contributes to neurodegeneration in Parkinson's disease. Neuron 109, 3758-3774 e3711 (2021).
A. M. Cuervo, L. Stefanis, R. Fredenburg, P. T. Lansbury, D. Sulzer, Impaired degradation of mutant alpha-synuclein by chaperone-mediated autophagy. Science 305, 1292-1295 (2004).
J. L. Webb, B. Ravikumar, J. Atkins, J. N. Skepper, D. C. Rubinsztein, Alpha-Synuclein is degraded by both autophagy and the proteasome. J Biol Chem 278, 25009-25013 (2003).
D. J. Klionsky, S. D. Emr, Autophagy as a regulated pathway of cellular degradation. Science 290, 1717-1721 (2000).
A. Simonsen, S. A. Tooze, Coordination of membrane events during autophagy by multiple class III PI3-kinase complexes. J Cell Biol 186, 773-782 (2009).

Although the invention has been described with reference to the above examples, it will be understood that modifications and variations are encompassed within the spirit and scope of the invention. Accordingly, the invention is limited only by the following claims.

1. An isolated nucleic acid sequence comprising:
a first nucleic acid sequence encoding an alpha-synuclein (α-syn) protein;
a second nucleic acid sequence encoding a light-responsive domain; and
a third nucleic acid sequence encoding a protein tag, in operable linkage.

2. The isolated nucleic acid sequence of claim 1, wherein the light-responsive domain is a Cry2 PHR or a Cry2clust light-responsive domain.

3. (canceled)

4. The isolated nucleic acid sequence of claim 1, wherein the light-responsive domain is fused at the C-terminus of the α-syn protein.

5-6. (canceled)

7. An isolated mammalian cell comprising a vector comprising the nucleic acid sequence of claim 1.

8. The isolated cell of claim 7, wherein the cell is a terminally differentiated neuron, an induced pluripotent stem cell-derived neural progenitor cell (iPSC NPC), or an iPSC-derived midbrain dopaminergic (iPSC-derived mDA) neuron.

9. A method of inducing aggregation of an alpha-synuclein (α-syn) protein in a cell comprising:
contacting the cell with a vector comprising the nucleic acid sequence of claim 1; and
exposing the cell to blue light illumination,
thereby inducing aggregation of α-syn protein in the cell.

10-11. (canceled)

12. The method of claim 9, wherein exposing the cell to blue light illumination comprises exposing the cell to an acute pulsed blue light stimulation at a light intensity about 26 μW/mm² to 34 μW/mm², frequency about 0.17 Hz, 0.25 HZ, 0.5 Hz or 1 Hz, and duration about 1 hour and 7 days, or a combination thereof.

13-21. (canceled)

22. The method of claim 9, wherein the α-syn aggregates are pathogenic α-syn aggregates.

23. The method of claim 9, wherein the α-syn aggregates comprise 5G4⁺, Syn-O2⁺, pS129⁺, Syn303⁺, p62⁺, ThioS⁺ and/or ubiquitin⁺ α-syn aggregates.

24. (canceled)

25. A method of identifying an α-syn aggregation inhibitor comprising:
(i) contacting a cell with a vector comprising the nucleic acid sequence of claim 1,
(ii) contacting the cell with a test compound,
(iii) exposing the cell from (ii) to blue light illumination, and
measuring an aggregate induction score (AIS) of the test compound in the cell exposed to blue light illumination (blue AIS); and
(iv) exposing a cell from (iii) to the dark and measuring an AIS of the test compound in the cell exposed to the dark (dark AIS),
wherein an α-syn aggregation inhibitor has a greater blue AIS as compared to a dark AIS, thereby identifying α-syn aggregation inhibitor.

26-34. (canceled)

35. The method of claim 25, wherein the α-syn aggregation inhibitor is selected from Cyclothiazide, BVT 948, CI 976, NE 100 hydrochloride, BX 471, C 021 dihydrochloride, BAG 956, Arcyriaflavin A, Amlexanox, Kartogenin, Neuropathiazol, Napabucasin, Dexamethasone, XD 14, Mycophenolic acid, Cilostazol, Mycophenolate mofetil, Rizatriptan benzoate, or AZD 1480.

36. A method of treating a synucleinopathy in a subject comprising:
administering to the subject in need thereof an α-syn aggregation inhibitor identified by the method of claim 25.

37. (canceled)

38. The method of claim 36, wherein the synucleinopathy is selected from the group consisting of Parkinson Disease (PD), dementia with Lewy body (DLB), multiple system atrophy (MSA), and neuroaxonal dystrophy.

39. An optogenetic alpha-synuclein (α-syn) aggregation system comprising:
(i) a LED illuminator; and
(ii) a cell comprising a vector comprising a nucleic acid sequence encoding an α-syn fusion protein.

40-42. (canceled)

43. A method of providing neuroprotective effects against a neurodegenerative disease in a subject comprising administering to the subject BAG 956, thereby providing neuroprotective effects.

44. The method of claim 43, wherein providing neuroprotective effects comprises inducing the clearance of alpha-synuclein (α-syn) aggregates, inducing the clearance of tau aggregates, and/or inducing autophagic flux and autophagic degradation of α-syn and/or tau aggregates.

45. The method of claim 43, wherein inducing the clearance of alpha-synuclein (α-syn) aggregates and/or inducing the clearance of tau aggregates comprises decreasing levels of insoluble pS129−, α-syn, pTau 202/205, pTau 231, pTau 217 and or AT8+ pTau in the subject.

46. The method of claim 44, wherein inducing autophagic flux and autophagic degradation of α-syn and/or tau aggregates comprises inhibiting PI3K/PDK1/AKT/mTor pathway in dopaminergic neurons, inducing LC3II expression, inhibiting p62 expression, and/or increasing LC3+ and/or LC3_5G4+ vesicles in dopaminergic neurons.

47-49. (canceled)

50. The method of claim 43, wherein administration of BAG 956 comprises oral administration of about 1-20 mg/kg.

51. (canceled)

52. The method of claim 43, wherein the neurodegenerative disease is selected from the group consisting of dementia with Lewy body (DLB), multiple system atrophy (MSA), neuroaxonal dystrophy, Alzheimer's disease, primary age-related tauopathy (PART), chronic traumatic encephalopathy (CTE), progressive supranuclear palsy (PSP), corticobasal degeneration (CBD), frontotemporal dementia and parkinsonism linked to chromosome 17 (FTDP-17), amyotrophic lateral sclerosis-parkinsonism-dementia (ALS-PDC, Lytico-bodig disease), ganglioglioma, gangliocytoma, meningioangiomatosis, postencephalitic parkinsonism, subacute sclerosing panencephalitis (SSPE), lead encephalopathy, tuberous sclerosis, pantothenate kinase-associated neurodegeneration, lipofuscinosis, and Parkinson's disease.

53. (canceled)


SEQUENCE LISTING (7 sequences)

SEQ ID NO: 1 - protein, 6 aa, synthetic construct
  REGION 1..6: note = consensus sequence
KTKEGV

SEQ ID NO: 2 - DNA, 24 bp, synthetic construct
  misc_feature 1..24: note = forward oligonucleotides
  source 1..24: mol_type = other DNA
caccgtcccctccaccccacagtg

SEQ ID NO: 3 - DNA, 24 bp, synthetic construct
  misc_feature 1..24: note = reverse oligonucleotides
  source 1..24: mol_type = other DNA
aaaccactgtggggtggaggggac

SEQ ID NO: 4 - DNA, 20 bp, synthetic construct
  misc_feature 1..20: note = forward primer
  source 1..20: mol_type = other DNA
ctgccgtctctctcctgagt

SEQ ID NO: 5 - DNA, 20 bp, synthetic construct
  misc_feature 1..20: note = reverse primer
  source 1..20: mol_type = other DNA
gtgggcttgtactcggtcat

SEQ ID NO: 6 - DNA, 20 bp, synthetic construct
  misc_feature 1..20: note = forward primer
  source 1..20: mol_type = other DNA
ttcgggtcacctctcactcc

SEQ ID NO: 7 - DNA, 20 bp, synthetic construct
  misc_feature 1..20: note = reverse primer
  source 1..20: mol_type = other DNA
ggctccatcgtaagcaaacc